SEQUENCE LISTING (165 sequences)

SEQ ID NO: 1 - protein, 526 aa, Artificial Sequence
  >SARS-CoV-2 Spike RBD-Fc
MSTMRLLTLALLFSCSVARAACRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 2 - protein, 22 aa, Artificial Sequence
  >Signal peptide of GluN1
MSTMRLLTLALLFSCSVARAAC

SEQ ID NO: 3 - protein, 273 aa, Severe acute respiratory syndrome coronavirus 2
  >RBD-SD1 region of SARS-CoV-2 Spike
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 4 - protein, 8 aa, Artificial Sequence
  >Linker between RBD-SD1 and rabbit Fc
GSSTMVRS

SEQ ID NO: 5 - protein, 223 aa, Artificial Sequence
  >rabbit Fc from vector pFuse-rIgG-Fc1
SKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 6 - protein, 633 aa, Artificial Sequence
  >ACE2-His-HA
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADHHHHHHHHGYPYDVPDYA

SEQ ID NO: 7 - protein, 615 aa, Homo sapiens
  >Amino acids 1-615 of ACE2 including signal peptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 8 - protein, 8 aa, Artificial Sequence
  >Polyhistidine tag
HHHHHHHH

SEQ ID NO: 9 - protein, 9 aa, Artificial Sequence
  >Hemagglutinin tag
YPYDVPDYA

SEQ ID NO: 10 - protein, 8 aa, Homo sapiens
  >HL CV07-250 H-CDR1
GYNFTNFA

SEQ ID NO: 11 - protein, 8 aa, Homo sapiens
  >HL CV07-250 H-CDR2
ISGYNGDT

SEQ ID NO: 12 - protein, 15 aa, Homo sapiens
  >HL CV07-250 H-CDR3
AGSDNYGFPYNGMDV

SEQ ID NO: 13 - protein, 9 aa, Homo sapiens
  >HL CV07-250 L-CDR1
SSDLGAYHF

SEQ ID NO: 14 - skipped

SEQ ID NO: 15 - protein, 10 aa, Homo sapiens
  >HL CV07-250 L-CDR3
SSYAGNNDFV

SEQ ID NO: 16 - protein, 471 aa, Homo sapiens
  >HL CV07-250 VH
MGWSCIILFLVATATGVHSQVQLVQSEAEVKEPGASVEVSCKASGYNFTNFAISWVRQAPGQGLEWMGWISGYNGDTNSAQKFLGRVTMTTDTSTTTAYMELRSLRSDDTAVYYCAGSDNYGFPYNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 235 aa, Homo sapiens
  >HL CV07-250 VL
MGWSCIILFLVATATGSWAQSALTQPPSASGSPGQSVTISCTGTSSDLGAYHFVTWYQHYPGKAPKVMIYGVRKRPSGVPDRFSGSKSGNTASLTVSGLQDEDEADYYCSSYAGNNDFVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 8 aa, Homo sapiens
  >HL CV07-270 H-CDR1
GFTFSDYY

SEQ ID NO: 19 - protein, 8 aa, Homo sapiens
  >HL CV07-270 H-CDR2
ISSSGSTI

SEQ ID NO: 20 - protein, 22 aa, Homo sapiens
  >HL CV07-270 H-CDR3
ARARGSSGWYRIGTRWGNWFDP

SEQ ID NO: 21 - protein, 9 aa, Homo sapiens
  >HL CV07-270 L-CDR1
SSDVGGYNY

SEQ ID NO: 22 - skipped

SEQ ID NO: 23 - protein, 10 aa, Homo sapiens
  >HL CV07-270 L-CDR3
SSYTSSSNVV

SEQ ID NO: 24 - protein, 478 aa, Homo sapiens
  >HL CV07-270 VH
MGWSCIILFLVATATGVHSQVQLVESGGGLVKPGGSLRLSCAASGFTFSDYYMTWIRQAPGKGLEWVSYISSSGSTIYYADSVKGRFTISRDNAKNSLYLQMNSLRAEDTAVYYCARARGSSGWYRIGTRWGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 235 aa, Homo sapiens
  >HL CV07-270 VL
MGWSCIILFLVATATGSWAQSALTQPASVSGSPGQSITISCTGTSSDVGGYNYVSWYQQHPGKAPKLMIYEVSNRPSGVSNRFSGSKSGNTASLTISGLQAEDEADYYCSSYTSSSNVVFGGGTMLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 8 aa, Homo sapiens
  >HL CV07-283 H-CDR1
GYTFTGYY

SEQ ID NO: 27 - protein, 8 aa, Homo sapiens
  >HL CV07-283 H-CDR2
INPISGGT

SEQ ID NO: 28 - protein, 18 aa, Homo sapiens
  >HL CV07-283 H-CDR3
VRGPFYYDSSGPLGGMDV

SEQ ID NO: 29 - protein, 9 aa, Homo sapiens
  >HL CV07-283 L-CDR1
SGDVGGYNY

SEQ ID NO: 30 - skipped

SEQ ID NO: 31 - protein, 10 aa, Homo sapiens
  >HL CV07-283 L-CDR3
SSYTSSSTYV

SEQ ID NO: 32 - protein, 474 aa, Homo sapiens
  >HL CV07-283 VH
MGWSCIILFLVATATGVHSQVQLVQSGAEVKKPGASAKVSCKASGYTFTGYYIHWVRQAPGQGLEWMGLINPISGGTNYAQKFQDRVTMTRDTSISTAYMELSRLRSDDTAVYYCVRGPFYYDSSGPLGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 235 aa, Homo sapiens
  >HL CV07-283 VL
MGWSCIILFLVATATGSWAQSALTQPASVSGSPGQSITISCTGTSGDVGGYNYVSWYQQHPGKAPKLMIYEVSNRPSGVSNRFSGSKSGNTASLTISGLQAEDEADYYCSSYTSSSTYVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 8 aa, Homo sapiens
  >HK CV07-287 H-CDR1
GFTFTSSA

SEQ ID NO: 35 - protein, 8 aa, Homo sapiens
  >HK CV07-287 H-CDR2
IVVGSGNT

SEQ ID NO: 36 - protein, 16 aa, Homo sapiens
  >HK CV07-287 H-CDR3
AAPYCSSTNCYDAFDI

SEQ ID NO: 37 - protein, 7 aa, Homo sapiens
  >HK CV07-287 L-CDR1
QSVSSSY

SEQ ID NO: 38 - skipped

SEQ ID NO: 39 - protein, 9 aa, Homo sapiens
  >HK CV07-287 L-CDR3
QQYGSSPWT

SEQ ID NO: 40 - protein, 472 aa, Homo sapiens
  >HK CV07-287 VH
MGWSCIILFLVATATGVHSQVQLVQSGPEVKKPGTSVKVSCKASGFTFTSSAMQWVRQARGQRLEWIGWIVVGSGNTNYAQKFQERVTITRDMSTSTAYMELSSLRSEDTAVYYCAAPYCSSTNCYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 234 aa, Homo sapiens
  >HK CV07-287 VL
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 8 aa, Homo sapiens
  >HK CV05-163 H-CDR1
GYTFTGYY

SEQ ID NO: 43 - protein, 8 aa, Homo sapiens
  >HK CV05-163 H-CDR2
INPNSGGT

SEQ ID NO: 44 - protein, 17 aa, Homo sapiens
  >HK CV05-163 H-CDR3
AREVMVRGALPPYGMDV

SEQ ID NO: 45 - protein, 6 aa, Homo sapiens
  >HK CV05-163 L-CDR1
QSVSSY

SEQ ID NO: 46 - skipped

SEQ ID NO: 47 - protein, 10 aa, Homo sapiens
  >HK CV05-1631 L-CDR3
QQRSNWPPVT

SEQ ID NO: 48 - protein, 473 aa, Homo sapiens
  >HK CV05-163 VH
MGWSCIILFLVATATGVHSQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREVMVRGALPPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 234 aa, Homo sapiens
  >HK CV05-163 VL
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 8 aa, Homo sapiens
  >HK CV07-209 H-CDR1
GFTFSDYY

SEQ ID NO: 51 - protein, 8 aa, Homo sapiens
  >HK CV07-209 H-CDR2
ISSSVSTI

SEQ ID NO: 52 - protein, 12 aa, Homo sapiens
  >HK CV07-209 H-CDR3
ARDGVIPPRFDY

SEQ ID NO: 53 - protein, 6 aa, Homo sapiens
  >HK CV07-209 L-CDR1
QDISNY

SEQ ID NO: 54 - skipped

SEQ ID NO: 55 - protein, 9 aa, Homo sapiens
  >HK CV07-209 L-CDR3
QQYDNLPLT

SEQ ID NO: 56 - protein, 468 aa, Homo sapiens
  >HK CV07-209 VH
MGWSCIILFLVATATGVHSQVQLVESGGGLVKPGGSLRLSCAASGFTFSDYYMSWIRQAPGKGLEWVSYISSSVSTIYYADSVKGRFTISRDNAKNSLYLQMNSLRAEDTAVYYCARDGVIPPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 233 aa, Homo sapiens
  >HK CV07-209 VL
MGWSCIILFLVATATGVHSDIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 8 aa, Homo sapiens
  >HL CV07-222 H-CDR1
GYTFTDYY

SEQ ID NO: 59 - protein, 8 aa, Homo sapiens
  >HL CV07-222 H-CDR2
INPISGGT

SEQ ID NO: 60 - protein, 18 aa, Homo sapiens
  >HL CV07-222 H-CDR3
ARGPYYYDSSGSLGAFDI

SEQ ID NO: 61 - protein, 8 aa, Homo sapiens
  >HL CV07-222 L-CDR1
SSDVGSYN

SEQ ID NO: 62 - skipped

SEQ ID NO: 63 - protein, 11 aa, Homo sapiens
  >HL CV07-222 L-CDR3
CSYAGGSTSYV

SEQ ID NO: 64 - protein, 474 aa, Homo sapiens
  >HL CV07-222 VH
MGWSCIILFLVATATGVHSQVQLVQSGAEVKKPGASVKVSCKASGYTFTDYYVHWVRQAPGQGLEWMGWINPISGGTNYAQKFQGRVTMTRDTSISTAYMELSRLRSDDTAVYYCARGPYYYDSSGSLGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 236 aa, Homo sapiens
  >HL CV07-222 VL
MGWSCIILFLVATATGSWAQSALTQPASVSGSPGQSITISCTGTSSDVGSYNLVSWYQQHPGKAPKLMIYEGSKRPSGVSNRFSGSKSGNTASLTISGLQAEDEAHYYCCSYAGGSTSYVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 8 aa, Homo sapiens
  >HL CV07-262 H-CDR1
GYTFTGYY

SEQ ID NO: 67 - protein, 8 aa, Homo sapiens
  >HL CV07-262 H-CDR2
INPNSGGT

SEQ ID NO: 68 - protein, 20 aa, Homo sapiens
  >HL CV07-262 H-CDR3
ARVGWYDGTGDYYYYYGMDV

SEQ ID NO: 69 - protein, 9 aa, Homo sapiens
  >HL CV07-262 L-CDR1
SSDVGSYNL

SEQ ID NO: 70 - skipped

SEQ ID NO: 71 - protein, 10 aa, Homo sapiens
  >HL CV07-262 L-CDR3
CSYAGTSTFV

SEQ ID NO: 72 - protein, 478 aa, Homo sapiens
  >HL CV07-262 VH
MGWSCIILFLVATATGVHSQVQLVQSGAEVKKPGASVKVSCKASGYTFTGYYMHWVRQAPGQGLEWMGWINPNSGGTNYAQTFQGRVTMTRDTSITTTYMELSRLRSDDTAVYYCARVGWYDFGTPGDYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 235 aa, Homo sapiens
  >HL CV07-262 VL
MGWSCIILFLVATATGSWAQSALTQPASVSGSPGQSITISCTGTSSDVGSYNLVSWYQQHPGKAPKLMIYEGSKRPSGVSNRFSGSKSGNTASLTISGLQAEDEADYFCCSYAGTSTFVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 8 aa, Homo sapiens
  >HK CV38-113 H-CDR1
GFTVSSNY

SEQ ID NO: 75 - protein, 7 aa, Homo sapiens
  >HK CV38-113 H-CDR2
IYSGGST

SEQ ID NO: 76 - protein, 13 aa, Homo sapiens
  >HK CV38-113 H-CDR3
ARGGRLADAAGDY

SEQ ID NO: 77 - protein, 6 aa, Homo sapiens
  >HK CV38-113 L-CDR1
QDISNY

SEQ ID NO: 78 - skipped

SEQ ID NO: 79 - protein, 10 aa, Homo sapiens
  >HK CV38-113 L-CDR3
QQYDNLPSWT

SEQ ID NO: 80 - protein, 468 aa, Homo sapiens
  >HK CV38-113 VH
MGWSCIILFLVATATGVHSEVQLVESGGGLVQPGGSLRLSCAASGFTVSSNYMSWVRQAPGKGLEWVSVIYSGGSTYYADSVKGRFTISRHNSKNTLYLQMNSLRAEDTAVYFCARGGRLADAAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 234 aa, Homo sapiens
  >HK CV38-113 VL
MGWSCIILFLVATATGVHSDIQMTQSPSSLSASVGDRVTITCQASQDISNYLNWYQQKPGKAPKLLIYDASNLETGVPSRFSGSGSGTDFTFTISSLQPEDIATYYCQQYDNLPSWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 8 aa, Homo sapiens
  >HL CV38-183 H-CDR1
GFTVSSNY

SEQ ID NO: 83 - protein, 7 aa, Homo sapiens
  >HL CV38-183 H-CDR2
IYSGGST

SEQ ID NO: 84 - protein, 15 aa, Homo sapiens
  >HL CV38-183 H-CDR3
ARGDGWDNYYYGMDV

SEQ ID NO: 85 - protein, 9 aa, Homo sapiens
  >HL CV38-183 L-CDR1
SSNIGAGYD

SEQ ID NO: 86 - skipped

SEQ ID NO: 87 - protein, 11 aa, Homo sapiens
  >HL CV38-183 L-CDR3
QSYDSSLSGSV

SEQ ID NO: 88 - protein, 470 aa, Homo sapiens
  >HL CV38-183 VH
MGWSCIILFLVATATGVHSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGDGWDNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 236 aa, Homo sapiens
  >HL CV38-183 VL
MGWSCIILFLVATATGSWAQSVLTQPPSVSGAPGQRVTISCTGSSSNIGAGYDVHWYQHLPGTAPKLLIYANSNRPSGVPDRFSGSKSGTSASLAITGLQAEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 8 aa, Homo sapiens
  >HL CV07-255 H-CDR1
GYTFTGYY

SEQ ID NO: 91 - protein, 8 aa, Homo sapiens
  >HL CV07-255 H-CDR2
INPNSGGT

SEQ ID NO: 92 - protein, 15 aa, Homo sapiens
  >HL CV07-255 H-CDR3
ARDSRFSYVNGEFDY

SEQ ID NO: 93 - protein, 9 aa, Homo sapiens
  >HL CV07-255 L-CDR1
SSDVESYNL

SEQ ID NO: 94 - skipped

SEQ ID NO: 95 - DNA, 10 bp, Homo sapiens
  >HL CV07-255 L-CDR3
csyaghstwv

SEQ ID NO: 96 - protein, 471 aa, Homo sapiens
  >HL CV07-255 VH
MGWSCIILFLVATATGVHSQVQLVQSGAEVKKPEASVKVSCKASGYTFTGYYMHWVRQAPGQGLEWMGWINPNSGGTNYAQKFQGRVTMTRDTSISTVYMELSRLRSDDTAVYYCARDSRFSYVNGEFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 235 aa, Homo sapiens
  >HL CV07-255 VL
MGWSCIILFLVATATGSWAQSALTQPASVSGSPGQSITISCTRTSSDVESYNLVSWYQQHPGKAPKLMIYEVSKRPSGISNRFSGSKSGNTASLTISGLQAEDEADYYCCSYAGHSTWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 98 - protein, 8 aa, Homo sapiens
  >HL CV07-315 H-CDR1
GFTFDDYA

SEQ ID NO: 99 - protein, 8 aa, Homo sapiens
  >HL CV07-315 H-CDR2
ISWNSGSI

SEQ ID NO: 100 - protein, 18 aa, Homo sapiens
  >HL CV07-315 H-CDR3
AKDFLWDLHPPRYYGMDV

SEQ ID NO: 101 - protein, 8 aa, Homo sapiens
  >HL CV07-315 L-CDR1
SSNIGNNY

SEQ ID NO: 102 - skipped

SEQ ID NO: 103 - protein, 10 aa, Homo sapiens
  >HL CV07-315 L-CDR3
GTWDSSLNGV

SEQ ID NO: 104 - protein, 474 aa, Homo sapiens
  >HL CV07-315 VH
MGWSCIILFLVATATGVHSEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDFLWDLHPPRYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 234 aa, Homo sapiens
  >HL CV07-315 VL
MGWSCIILFLVATATGSWAQSVLTRPPSVSAAPGQKVTISCSGSSSNIGNNYVSWYQQLPGTAPKLLIYENNKRPSGIPDRFSGSKSGTSATLGITGLQTGDEADYYCGTWDSSLNGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 106 - protein, 8 aa, Homo sapiens
  >HK CV38-139 H-CDR1
GFTVSSNY

SEQ ID NO: 107 - protein, 7 aa, Homo sapiens
  >HK CV38-139 H-CDR2
FYSGGST

SEQ ID NO: 108 - protein, 10 aa, Homo sapiens
  >HK CV38-139 H-CDR3
ARGHYDLFDY

SEQ ID NO: 109 - protein, 6 aa, Homo sapiens
  >HK CV38-139 L-CDR1
QGISSY

SEQ ID NO: 110 - skipped

SEQ ID NO: 111 - protein, 10 aa, Homo sapiens
  >HK CV38-139 L-CDR3
QQLNSYPPGT

SEQ ID NO: 112 - protein, 465 aa, Homo sapiens
  >HK CV38-139 VH
MGWSCIILFLVATATGVHSEVQLVESGGGLVQPGGSLRLSCAASGFTVSSNYMSWVRQAPGKGLEWVSVFYSGGSTFYADSVKGRFTISRDNSKNTLYLQMNSLRAEDTAVYYCARGHYDLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 234 aa, Homo sapiens
  >HK CV38-139 VL
MGWSCIILFLVATATGVHSDIQLTQSPSFLSASVGDRVTITCRASQGISSYLAWYQQKPGKAPKLLIYAASTLQSGVPSRFSGSGSGTEFTLTISSLQPEDFATYYCQQLNSYPPGTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 8 aa, Homo sapiens
  >HK CV38-142 H-CDR1
GYSFTSYW

SEQ ID NO: 115 - protein, 8 aa, Homo sapiens
  >HK CV38-142 H-CDR2
IYPGESDT

SEQ ID NO: 116 - protein, 16 aa, Homo sapiens
  >HK CV38-142 H-CDR3
ARIRGVYSSGWIGGDY

SEQ ID NO: 117 - protein, 6 aa, Homo sapiens
  >HK CV38-142 L-CDR1
QSISSY

SEQ ID NO: 118 - skipped

SEQ ID NO: 119 - protein, 11 aa, Homo sapiens
  >HK CV38-142 L-CDR3
QQSYSTPRQWT

SEQ ID NO: 120 - protein, 472 aa, Homo sapiens
  >HK CV38-142 VH
MGWSCIILFLVATATGVHSEVQLVQSGAEVKKPGESLKISCQGSGYSFTSYWIGWVRQMPGKGLEWMGIIYPGESDTRYSSSFQGHVTISADKSISTAYLQWSSLKASDTAMYYCARIRGVYSSGWIGGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 235 aa, Homo sapiens
  >HK CV38-142 VL
MGWSCIILFLVATATGVHSDIQMTQSPSSLSASVGDRVTITCRASQSISSYLNWYQQKPGKAPKLLIYAASSLQSGVPSRFSGSGSGTDFTLTISSLQPEDFATYYCQQSYSTPRQWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 8 aa, Homo sapiens
  >HK CVX1-126 H-CDR1
GLTVSSNY

SEQ ID NO: 123 - protein, 7 aa, Homo sapiens
  >HK CVX1-126 H-CDR2
IYSGGST

SEQ ID NO: 124 - protein, 11 aa, Homo sapiens
  >HK CVX1-126 H-CDR3
ARDLSEGGMDV

SEQ ID NO: 125 - protein, 6 aa, Homo sapiens
  >HK CVX1-126 L-CDR1
QGISSW

SEQ ID NO: 126 - skipped

SEQ ID NO: 127 - protein, 9 aa, Homo sapiens
  >HK CVX1-126 L-CDR3
QQANGFPPL

SEQ ID NO: 128 - protein, 466 aa, Homo sapiens
  >HK CVX1-126 VH
MGWSCIILFLVATATGVHSEVQLVESGGGLVQPGGSLRLSCAASGLTVSSNYMSWVRQAPGKGLEWVSVIYSGGSTYYADSVKGRFTISRHNSKNTLYLQMNSVRAEDTAVYYCARDLSEGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 233 aa, Homo sapiens
  >HK CVX1-126 VL
MGWSCIILFLVATATGVHSDIQMTQSPSSVSASVGDRVTITCRASQGISSWLAWYQQKPGKAPKLLIYAASTLQSGVPSRFSGSGSGTDFTLTISSLQPEDFATYYCQQANGFPPLFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 8 aa, Homo sapiens
  >HK CV38-221 H-CDR1
GITVSSNY

SEQ ID NO: 131 - protein, 7 aa, Homo sapiens
  >HK CV38-221 H-CDR2
IYSGGST

SEQ ID NO: 132 - protein, 11 aa, Homo sapiens
  >HK CV38-221 H-CDR3
ARGFGDYYFDY

SEQ ID NO: 133 - protein, 7 aa, Homo sapiens
  >HK CV38-221 L-CDR1
QSVSSSY

SEQ ID NO: 134 - skipped

SEQ ID NO: 135 - protein, 5 aa, Homo sapiens
  >HK CV38-221 L-CDR3
QQLYT

SEQ ID NO: 136 - protein, 466 aa, Homo sapiens
  >HK CV38-221 VH
MGWSCIILFLVATATGVHSEVQLVESGGGLVQPGGSLRLSCAASGITVSSNYMIWVRQAPGKGLEWVSVIYSGGSTYYADSVKGRFTISRDNSKNTLYLQMNSLRAEDTAVYYCARGFGDYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 230 aa, Homo sapiens
  >HK CV38-221 VL
MGWSCIILFLVATATGVHSEIVLTQSPGTLSLSPGERATLSCRASQSVSSSYFAWYQQKPGQAPRLLIYGASSRATGIPDRFSGSGSGTDFTLTISRLEPEDFAVYYCQQLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 8 aa, Homo sapiens
  >HK CVX2-106 H-CDR1
GGTFSSYA

SEQ ID NO: 139 - protein, 8 aa, Homo sapiens
  >HK CVX2-106 H-CDR2
IIPIFGTA

SEQ ID NO: 140 - protein, 18 aa, Homo sapiens
  >HK CVX2-106 H-CDR3
ATRKETTVTTSLVYGMDV

SEQ ID NO: 141 - protein, 6 aa, Homo sapiens
  >HK CVX2-106 L-CDR1
QSISSF

SEQ ID NO: 142 - skipped

SEQ ID NO: 143 - protein, 9 aa, Homo sapiens
  >HK CVX2-106 L-CDR3
QQSYSTPYT

SEQ ID NO: 144 - protein, 474 aa, Homo sapiens
  >HK CVX2-106 VH
MGWSCIILFLVATATGVHSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCATRKETTVTTSLVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 233 aa, Homo sapiens
  >HK CVX2-106 VL
MGWSCIILFLVATATGVHSDIQMTQSPSSLSASVGDRVTITCRASQSISSFLNWYQQKPGKAPKLLIYAASSLQSGVPSRFSGSGSVTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 8 aa, Homo sapiens
GFTVSSNY

SEQ ID NO: 147 - protein, 7 aa, Homo sapiens
IYSGGST

SEQ ID NO: 148 - protein, 11 aa, Homo sapiens
ARDLYYYGMDV

SEQ ID NO: 149 - protein, 6 aa, Homo sapiens
QGISSY

SEQ ID NO: 150 - skipped

SEQ ID NO: 151 - protein, 9 aa, Homo sapiens
QQLNNYSVT

SEQ ID NO: 152 - protein, 466 aa, Homo sapiens
MGWSCIILFLVATATGVHSEVQLVESGGGLIQPGGSLRLSCAASGFTVSSNYMSWVRQAPGKGLEWISVIYSGGSTFYADSVKGRFTISRDNSKNTLYLQMNSLRAEDTAVYYCARDLYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 233 aa, Homo sapiens
MGWSCIILFLVATATGVHSDIQLTQSPSFLSASVGDRVTITCRASQGISSYLAWYQQKPGKAPKLLIYAASTLQSGVPSRFSGSGSGTEFTLTISSLQPEDFATYYCQQLNNYSVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 8 aa, Homo sapiens
GYTFTGYY

SEQ ID NO: 155 - protein, 8 aa, Homo sapiens
INPNSGGT

SEQ ID NO: 156 - protein, 22 aa, Homo sapiens
ARVFGPGLDCSSTSCYTYGMDV

SEQ ID NO: 157 - protein, 9 aa, Homo sapiens
SSDVGSYNL

SEQ ID NO: 158 - skipped

SEQ ID NO: 159 - protein, 10 aa, Homo sapiens
CSYAGSSSWV

SEQ ID NO: 160 - protein, 478 aa, Homo sapiens
MGWSCIILFLVATATGVHSQVQLVQSGAEVKKPGASVKVSCKASGYTFTGYYMHWVRQAPGQGLEWMGWINPNSGGTNSAQKFQGRVTMTRDTSITTAYMELSRLRSDDTAVYYCARVFGPGLDCSSTSCYTYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 235 aa, Homo sapiens
MGWSCIILFLVATATGSWAQSALTQPASVSGSPGQSITISCTGTSSDVGSYNLVSWYQQHPGKAPKLMIYEVSKRPSGVSNRFSGSKSGNTASLTISGLQAEDEADYYCCSYAGSSSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 162 - protein, 273 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCS

SEQ ID NO: 163 - protein, 526 aa, Artificial Sequence
  Spike-FC fusion
MSTMRLLTLALLFSCSVARAACRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 164 - protein, 273 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 165 - protein, 526 aa, Artificial Sequence
  Spike-Fc fusion
MSTMRLLTLALLFSCSVARAACRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2024/0376180 A1
(43) Pub. Date: Nov. 14, 2024

(54) HUMAN RECOMBINANT MONOCLONAL ANTIBODY AGAINST SARS-COV-2 SPIKE GLYCOPROTEIN

(71) Applicants: Deutsches Zentrum fur Neurodegenerative Erkrankungen e.V. (DZNE), Bonn (DE); Charite - Universitatsmedizin Berlin, Berlin (DE)

(72) Inventors: Jakob KREYE, Potsdam (DE); Harald PRUSS, Berlin (DE); S. Momsen REINCKE, Berlin (DE); Hans-Christian KORNAU, Berlin (DE); Elisa SANCHEZ-SENDIN, Berlin (DE); Daniel WENDISCH, Berlin (DE); Marcel Alexander MULLER, Berlin (DE); Leif Erik SANDER, Berlin (DE); Victor Max CORMAN, Berlin (DE)

(73) Assignees: Deutsches Zentrum fur Neurodegenerative Erkrankungen e.V. (DZNE), Bonn (DE); Charite - Universitatsmedizin Berlin, Berlin (DE)

(21) Appl. No.: 17/928,013

(22) PCT Filed: May 28, 2021
(86) PCT No.: PCT/EP2021/064352

§ 371 Date: Nov. 28, 2022

(30) Foreign Application Priority Data
May 29, 2020 (EP) 20177437.9
Jun. 24, 2020 (EP) 20182069.3

(51) Int. Cl. C07K 16/10; A61K 39/00; A61P 31/14

(52) U.S. Cl. CPC C07K 16/1003; A61P 31/14; A61K 2039/505; C07K 2317/14; C07K 2317/565; C07K 2317/76; C07K 2317/92

(57) ABSTRACT

The invention relates to a human recombinant monoclonal antibody or antibody fragment that binds the receptor binding domain (RBD) of the S1 subunit (S1) of a Spike glycoprotein of SARS-CoV-2, wherein the heavy and light chain variable amino acid sequences of the antibody (VH and VL) were isolated from convalescent COVID-19 patients. In further aspects, the invention relates to a nucleic acid molecule encoding an antibody or antibody fragment of the invention, a host cell comprising the nucleic acid molecule, and a pharmaceutical composition comprising an antibody or antibody fragment of the invention. The invention further relates to corresponding medical uses and therapeutic methods of administering an antibody or antibody fragment of the invention in the treatment and/or prevention of a medical condition associated with a SARS Coronavirus, in addition to diagnostic uses and methods.

The invention relates to the fields of therapeutic and diagnostic reagents for treating and/or diagnosing medical conditions associated with a SARS Coronavirus (SARS-CoV), such as COVID-19, and to therapeutic and/or diagnostic antibodies.

The invention relates to a human recombinant monoclonal antibody or antibody fragment that binds the receptor binding domain (RBD) of the S1 subunit (S1) of a recombinant Spike glycoprotein of SARS-CoV-2 expressed from human embryonic kidney (HEK293) cells (HEK-SARS-CoV-2-Spike-S1-RBD glycoprotein), wherein the heavy and light chain variable amino acid sequences of the antibody (VH and VL) were isolated from convalescent COVID-19 patients.

The invention further relates to an antibody or antibody fragment that binds the receptor binding domain (RBD) of a Spike glycoprotein of SARS-CoV-2, for example from a SARS-CoV-2 identified as an infectious virus in a human population, wherein the heavy and light chain variable amino acid sequences of the antibody (VH and VL) were isolated from convalescent COVID-19 patients.

The invention further relates to an antibody or antibody fragment that binds the receptor binding domain (RBD) of a Spike glycoprotein of a SARS-CoV-2 virus, said antibody or fragment thereof defined by the amino acid sequences, such as CDR and/or VH or VL sequences, of the antibodies disclosed herein.

In further aspects, the invention relates to a nucleic acid molecule comprising a nucleotide sequence that encodes an antibody or antibody fragment of the invention, a host cell comprising said nucleic acid molecule, a host cell capable of producing an antibody or antibody fragment of the invention and a pharmaceutical composition comprising an antibody or antibody fragment of the invention.

The invention further relates to the medical use and corresponding therapeutic methods of administering an antibody or antibody fragment of the invention in the treatment and/or prevention of a medical condition associated with a SARS Coronavirus, in addition to diagnostic uses and methods, such as an in vitro method for determining the presence or absence of SARS-CoV-2 viral protein in a sample using an antibody or antibody fragment of the invention.

### BACKGROUND OF THE INVENTION

Severe acute respiratory syndrome (SARS) is a Coronavirus (CoV) mediated respiratory disease which was first observed in 2002. Based on scientific reports it is assumed that all human CoVs may be of zoonotic origin. Once a human becomes infected, the virus can quickly spread via droplet transmission and close contact between humans, leading to epidemic scenarios or even to a pandemic.

As one example, “Coronavirus Disease 2019” (COVID-19) is caused by the pathogenic corona virus SARS-CoV-2. Beginning in December 2019, the virus spread globally within a few weeks and led to an international health emergency. The worldwide pandemic poses huge challenges to health systems and leads additionally to restrictions in social life and weakening of global market economies. Since no effective therapy is available to date, and the disease is associated with high morbidity and mortality, there is a great need for therapeutic interventions for those who are ill as well as for prophylactic measures to contain outbreaks.

From a pathophysiological point of view, SARS is a complex medical condition, where the virus starts replicating in the upper respiratory tract and can spread to the lower respiratory tract or target non-respiratory organs and cells. Typical symptoms can be fever, chills, dry cough, dyspnea and diarrhea, although symptoms may first appear 10-14 days post-infection. Clinical investigations show that also liver, kidney, heart, intestine, brain and lymphocytes can, in addition to the lung, be affected.

It is proposed that the virus directly promotes cell damage, whereby a systemic inflammatory response followed by multiple organ injury is triggered. CoVs also cause a dysfunctional renin-angiotensin system, which increases the pulmonary vascular permeability being a factor for the development pulmonary edema. Both systemic inflammatory responses and a dysfunctional renin-angiotensin system contribute to a so-called cytokine storm that triggers an acute respiratory distress syndrome (ARDS). Multi-organ failure and/or the onset of ARDS represent a severe state of SARS patients with a high mortality risk within few weeks or days.

The entry of SARS-CoV-2 into an infected cell is mediated by binding of the viral spike protein via its receptor binding domain (RBD) to the human angiotensin converting enzyme-2 (ACE2) target receptor. Blocking this interaction by human antibodies leads to a neutralization of the virus in patients and thus to a healing of the infection (Hoffmann 2020).

Neutralizing antibodies against SARS-CoV-2 have been described previously (Jiang 2020). Virus neutralizing antibodies play crucial roles in controlling viral infection. Currently developed SARS-CoV- and MERS-CoV-specific Abs include monoclonal antibodies (mAbs), their functional antigen-binding fragments, single-chain variable region fragments or single-domain antibodies (nanobodies). They target S1-RBD, S1-NTD, or the S2 region, blocking the binding of RBDs to their respective receptors and interfering with S2-mediated membrane fusion or entry into the host cell, thus inhibiting viral infections.

Therefore, various SARS-CoV- and MERS-CoV-mAbs have been described in the art in the context of their potential cross-neutralizing activity against SARS-CoV-2 infection. Very recently, further reports on anti-CoV-Spike antibodies isolated from convalescent patients post COVID-19 disease are also appearing, although additional research is required to assess these early reports (Ju et al, 2020, Robbiani, et al, 2020, Cao et al, 2020, Chen Xiangyu et al, 2020, Shi Rui et al, 2020, Zost et al, 2020). Other human monoclonal SARS-CoV-2neutralizing antibodies, chimeric antibodies or single-domain antibodies have been disclosed in the art (Wang et al, 2020, Wu Yanling et al, 2020). Upon review of the antibodies disclosed in these preliminary reports, it appears that one or more of the antibody properties, with respect to plaque reduction neutralization (as shown in a PRNT), affinity of the antibodies to the RBD epitope, an inhibition of ACE2 binding to Spike protein, potential unspecific cross-reaction to other tissue types, the in vivo properties of the antibodies, for example in animal models of SARS-CoV infection and COVID disease, or the ability to bind and neutralize multiple SARS-CoV-2 variants or mutants, are suboptimal.

The present invention demonstrates the isolation, cloning and characterization of novel human monoclonal antibodies that bind and neutralize the SARS-CoV-2 virus, suitable for therapeutic, prophylactic and diagnostic applications. The identification and recombinant production of such neutralizing antibodies from convalescent patients after COVID-19 infection, having been isolated using CoV-2-Spike-RBD protein recombinantly produced in HEK293 cells, enables strategies for therapy, prophylaxis and diagnosis of disease.

### SUMMARY OF THE INVENTION

In light of the prior art, the technical problem underlying the invention was the provision of an alternative or improved agent suitable as a therapeutic, prophylactic and/or diagnostic reagent for treating and/or diagnosing medical conditions associated with a SARS Coronavirus.

A further object of the invention was the provision of an antibody or antibody fragment, with improved SARS-CoV-2 neutralization properties. A further object of the invention was the provision of an antibody or antibody fragment, with improved affinity to the RBD epitope. A further object of the invention was the provision of an antibody or antibody fragment, with improved inhibition of ACE2 binding to Spike protein. A further object of the invention was the provision of an antibody or antibody fragment, with reduced unwanted, unspecific cross-reaction to other tissue types. A further object of the invention was the provision of an antibody or antibody fragment, with improved vivo efficacy in the treatment and/or prophylaxis of SARS coronavirus associated disease. A further object of the invention was the provision of an antibody or antibody fragment, with improved properties with respect to binding and/or neutralizing multiple SARS-CoV-2 variants or mutants.

This problem is solved by the features of the independent claims. Preferred embodiments of the present invention are provided by the dependent claims.

The invention therefore relates to a human recombinant monoclonal antibody or antibody fragment that binds the receptor binding domain (RBD) of the S1 subunit (S1) of a recombinant Spike glycoprotein of SARS-CoV-2 expressed from human embryonic kidney (HEK293) cells (HEK-SARS-CoV-2-Spike-S1-RBD glycoprotein), wherein the heavy and light chain variable amino acid sequences of the antibody (VH and VL) were isolated from convalescent COVID-19 patients.

The invention further relates to an antibody or antibody fragment that binds the receptor binding domain (RBD) of a Spike glycoprotein of SARS-CoV-2, for example from a SARS-CoV-2 identified as an infectious virus in a human population, wherein the heavy and light chain variable amino acid sequences of the antibody (VH and VL) were isolated from convalescent COVID-19 patients.

The invention further relates to an antibody or antibody fragment that binds the receptor binding domain (RBD) of a Spike glycoprotein of a SARS-CoV-2 virus, said antibody or fragment thereof defined by the amino acid sequences, such as CDR and/or VH or VL sequences, of the antibodies disclosed herein.

As described at length herein, the identification and recombinant production of Coronavirus neutralizing antibodies from convalescent patients after COVID-19 infection represents a novel and advantageous approach towards developing an effective Coronavirus therapeutic.

The approach employed avoids the need for antibody production in an animal, such as a mouse, and subsequent humanization, which is inherently fraught with difficulties in maintaining antibody binding properties, such as affinity and specificity, and reducing immunogenicity. The approach of the present invention employs the isolation of anti-Coronavirus antibodies from patients who have survived the disease, thereby inherently selecting for antibodies that were involved in successful immune response against viral infection.

In some embodiments, the means for isolation of the antibodies of the present invention are therefore a feature in characterizing the antibody properties. The functional property of having conveyed effective viral immunity against the SARS-Cov-2 represents a special technical feature that not only provides unique and advantageous properties to the antibodies as claimed, but additionally represents a unifying functional feature of the antibodies disclosed herein.

The novel monoclonal antibodies described herein enable treatment of a disease, for which the medical community-in the context of the 2019-2020 SARS-CoV-2 pandemic—is in desperate need of means to reduce infections and treat patients at risk of adverse outcomes.

At present, there is no effective medication available. Experiments with convalescent plasma are being conducted, although several disadvantages exist in this approach, including the use of an undefined product, which is difficult to produce and has very limited availability. Convalescent plasma also carries the risk of side effects due to undesirable factors, e.g. clotting factors (thromboses) or virus transmission (with plasma). There is a high individual variability through different donors and a potentially low potency due to useless or even harmful antibodies.

In contrast to convalescent plasma therapies, the antibodies described herein represent a high-purity reagent, comprising monoclonal antibodies of defined composition with a testable mechanism of action, which lead to a reduction of disease progression and severity and the prevention of virus spread. The fully human antibodies described herein comprise no unwanted components, will likely show no increased risk of thrombosis or of infection and clinicians can rely on extensive clinical experience with other monoclonal antibodies. The present invention offers the further advantages of a defined and standardized composition, good reproducibility, constant dosages, GMP standards and a high potency of antibodies after selection in preclinical testing.

The invention described herein comprises therefore the recombinant production and functional characterization of human monoclonal antibodies capable of neutralizing SARS-CoV-2. The properties of the antibodies and the corresponding sequences are described below.

The antibodies may be applied as therapeutic monoclonal antibodies or as a combination of multiple monoclonal antibodies in treating acute infection. As a therapeutic agent in acute COVID-19 infection, the neutralizing monoclonal antibodies of the present invention can help the patient's immune system to fight the virus by preventing the infection of further cells by the SARS-CoV-2 virus, and virus-infected cells can be labeled and eliminated by the immune system. Examples from other infectious diseases where this principle has been successfully applied are Ebola and HIV.

The invention further comprises the use of an antibody of the present invention as a prophylactic monoclonal antibody for pre-and/or post-exposure prophylaxis of disease. The neutralizing monoclonal antibodies described herein can be used as a prophylactic in groups of patients at risk of COVID-19 infection (such as health care workers), persons at risk of infection after proven contact to an infected individual, in patients who have been infected but do not yet show disease symptoms, or patients at high risk of a severe course or adverse event (e.g. patients with cancer or immunosuppression).

The invention further comprises the use of the SARS-CoV-2 antibodies described herein in order to develop and carry out a diagnostic assay.

In one embodiment of the invention, the heavy and light chain variable amino acid sequences of the antibody (VH and VL) were isolated from antibody-secreting cells (ASCs) and/or memory B cells (MBCs) of convalescent COVID-19 patients that bind the HEK-SARS-CoV-2-Spike-S1-RBD glycoprotein.

In one embodiment of the invention, the heavy and light chain variable amino acid sequences of the antibody (VH and VL) were isolated from CD19+CD27+CD38+ antibody-secreting cells (ASCs) and/or CD19+CD27+ memory B cells (MBCs) of convalescent COVID-19 patients that bind the HEK-SARS-CoV-2-Spike-S1-RBD glycoprotein.

As described below, the inventors employed a unique isolation procedure in order to selectively bind antibody-secreting cells and/or memory B cells that presented anti-Spike antibodies by using HEK-expressed recombinant Spike protein, which is then coupled to a label (fluorophore), which specifically binds those B-cells that bind the target Spike protein on the surface via their B-cell receptors/antibodies. Labelled cells were then sorted using FACS, isolated as single cells and from single cell cDNA, recombinant monoclonal antibodies (mAbs) were generated using a nested PCR strategy to amplify the variable domains of immunoglobulin (Ig) heavy and light chain genes.

In some embodiments, the antibodies of the present invention are therefore defined by the process of their isolation. The features of the method employed in their isolation lead to inherent antibody properties, namely the binding to a Spike protein form expressed in human cells and the additional benefit that the antibodies isolated from the patients were directly involved in a successful immune response to the Coronavirus. The features of the method of their production therefore inherently represent an important set of functional properties of the antibodies of the invention.

In one embodiment, the HEK-SARS-CoV-2-Spike-S1-RBD glycoprotein comprises or consists of an amino acid sequence according to SEQ ID NO 3.

This protein was recombinantly produced in HEK293 cells in order to obtain a glycosylation pattern as similar as possible to that seen in humans after infection or during infection, or as may be found in humans, for example in the blood. The antibodies of the present invention are therefore isolated via their binding to a target protein that mimics very closely the in vivo situation during infection.

This represents a significant advantage over other anti-Spike protein antibodies that were isolated via binding to Spike protein produced in non-human expression systems, such as in insect cells. The glycosylation obtained in human cells, preferably HEK cells, is therefore an important feature in defining the binding properties and the methods of isolation of the antibodies of the invention.

In some embodiments, a skilled person is capable of defining the glycosylation pattern of Spike-RBD when produced in any given cell type. The antigen, with which the inventive antibodies were isolated, therefore can play a role in determining the characteristics of the antibody and may represent a novel feature over the prior art. Methods for determining glycosylation of any given protein are described below and known to a skilled person.

In one embodiment, the antibody or antibody fragment inhibits the interaction between angiotensin-converting enzyme 2 (ACE2) or fragment thereof with the SARS-CoV-2-Spike-S1-RBD.

In one embodiment, the antibody or antibody fragment inhibits the interaction between angiotensin-converting enzyme 2 (ACE2) or fragment thereof according to SEQ ID NO 7 with the SARS-CoV-2-Spike-S1-RBD.

As described in detail below, the viral Spike protein binds the virus to its host cell via a receptor expressed by the host cell, namely the angiotensin-converting enzyme 2 (ACE2).

In some embodiments, the antibodies of the invention are capable of disrupting the interaction between ACE2 and the RBD of the Spike protein. Therefore, infection of a host cell can be prevented using the neutralizing antibodies of the present invention.

In some embodiments, the inhibition of interaction between a soluble fragment of ACE2 (preferably according to SEQ ID NO 7) and SARS-CoV-2-Spike-S1-RBD (preferably according to SEQ ID NO 3) obtained by the antibody or antibody fragment is at least about 5%, 10%, 15%, 20%, 25%, or preferably 30%, 35%, 40%, 45%, more preferably 50% or 55%, or more preferably at least about 60%, 70%,75%, 80%, 85%, 90% or 95% or more, in an in vitro competition assay in which HEK-SARS-CoV-2-Spike-S1-RBD glycoprotein is immobilized on a solid phase and incubated with the antibody or antibody fragment and subsequently with ACE2.

The assay described here is one example of how the binding of the Spike RBD and ACE2 can be assessed, preferably quantitatively or semi-quantitatively. In the examples below, an assay is described in which the RBD of the Spike glycoprotein is immobilized on a solid phase and first incubated with an antibody of the invention, and subsequently incubated with labeled ACE2. By detecting the amount of labeled ACE2 bound to the plate, e.g. after washing, it can be determined whether the ACE2 was able to bind the Spike protein that had been treated with antibody.

In an alternative assay setup ACE2 is bound to a solid phase and Spike protein treated with antibody is then incubated with the immobilized target ACE2.

Additional assays may be employed, for example where Spike protein, or Spike-RBD is expressed from a cell, and when presented on the cell surface, binding to Spike protein can be assessed. Examples of such an Assay are provided in Ju 2020, for example where Spike protein is expressed in HEK cells and antibody binding to the Spike protein is assessed. The cell type employed in such an assay may be selected appropriately, and may in some embodiments mimic the cells infected by CoV, such as endothelial cells, in particular cells in the vascular endothelia, kidney, bladder, heart, nasal mucosa, bronchus and/or lung cells.

The quantitative readout of the assay allows an objective measure of inhibition of ACE2-Spike interaction, which provides a therapeutically relevant functional definition of the antibody of the present invention. In some embodiments, the levels of inhibition are measured relative to a control in which no antibody—or a control antibody with a different specificity—was added with Spike protein and immobilized ACE2. Measurements in comparison to appropriate controls, independent of assay setup, may be easily determined and established by a skilled person.

In some embodiments, the viral particles are to be neutralized in the body of a patient infected with the virus. Therefore, binding properties of the Spike protein to ACE2, expressed on the cell surface of cardiomyocytes and/or endothelial cells, can be assessed using the assays described herein, when adjusted appropriately, which is within the ability of a skilled person.

By way of example, exemplary antibodies as described herein, that effectively inhibit the interaction between angiotensin-converting enzyme 2 (ACE2) with the SARS-CoV-2-Spike protein, are CV07-250, CV38-183 and CV07-209. Further exemplary antibodies with this beneficial property are described in the examples below.

In one embodiment, the antibody or antibody fragment inhibits the infection of epithelial cells (preferably kidney epithelial cells, such as VeroE6-cells) with SARS-CoV-2.

In some embodiments, the antibodies of the invention are capable of disrupting the interaction between ACE2 and the RBD of the Spike protein. Therefore, infection of a host cell can be prevented using the neutralizing antibodies of the present invention.

In some embodiments, the antibody or antibody fragment has an IC50 of <500 ng/mL, preferably <50 ng/mL, and/or an IC90 of <500 ng/ml, in a plaque reduction neutralization test (PRNT) (preferably using human epithelial cells, more preferably kidney epithelial cells, such as VeroE6-cells).

In some embodiments, the antibody or antibody fragment has an IC50 of <5000 ng/mL, preferably <2000 ng/mL, <1500 ng/mL, <1000 ng/mL, more preferably <500 ng/mL, <400 ng/mL, <300 ng/mL, <200 ng/mL, more preferably <100 ng/mL or <50 ng/mL. In some embodiments, the antibody or antibody fragment has an IC50 of <40 ng/mL, <25 ng/mL, <10 ng/mL, <5 ng/mL, or preferably <1 ng/mL.

In some embodiments, the function of preventing Coronavirus infection of an epithelial cell can be used to define the antibodies of the invention and represents a quantitative or semi-quantitative property of the antibodies that is a unique feature of the invention. As described in detail in the examples below, the ability of Coronavirus after antibody treatment to infect epithelial cells can be assessed using the techniques disclosed herein. This infectivity can be quantified, both with and without an antibody of the invention, and the rate of infection can be determined.

The IC50 and IC90 values used herein relate to their meaning commonly used in the art, and represent a concentration of antibody, for example expressed as ng/mL, that cause inhibition in a biological assay. The half maximal inhibitory concentration (IC50) is a measure of the potency of a substance in inhibiting a specific biological or biochemical function, in the present case the inhibition of Coronavirus infection of an epithelial cell in an in vitro assay of infection, as disclosed below. The IC50 is a quantitative measure that indicates how much of a particular inhibitory substance (e.g. drug) is needed to inhibit, in vitro, a given biological process or biological component (Coronavirus infection of an epithelial cell) by 50%. The IC90 corresponds to the same measurement and assay but is the concentration of antibody that achieves 90% inhibition of infection. IC50 and IC90 values can be determined by a skilled person without undue effort. Details on the epithelial infection assay are provided in the examples below.

By way of example, exemplary antibodies as described herein, that effectively inhibit the infection of epithelial cells with SARS-CoV-2 and/or effectively neutralize the virus to prevent infection, are CV07-250, CV38-183, CV07-209, or CV38-142. Further exemplary antibodies with this beneficial property are described in the examples below.

In one embodiment, the antibody or antibody fragment does not bind, or binds at negligible levels, unfixed mammalian tissue sections in vitro, said sections preferably selected from one or more of brain, heart, kidney, liver, gut and/or lung tissue sections.

As described in detail below, the inventors assessed the cross-reactivity of selected antibodies of the present invention for binding to murine tissue sections from different organs (incl. brain, heart, kidney, liver, gut and lung). The inventors identified autoreactive binding patterns from some of the antibodies, especially against brain tissue. This finding indicates that anti-Spike antibodies may also cross react and bind (auto-reactively) to tissue of the subject. This represents an unwanted property of such antibodies to be avoided in clinical practice. The invention therefore relates to antibodies that preferably do not exhibit such cross-reactive binding properties or exhibit such cross-reactivity at low or negligible levels, for example at a level that is unlikely to induce any adverse health effect in a patient.

The testing of this property has introduced an additional step in the isolation and selection process, which has not been previously proposed in the art. The finding that anti-Spike antibodies in convalescent patients may be cross-reactive against e.g. brain tissue is a surprising and unexpected discovery, which enables improved screening of candidate antibodies. In some embodiments, the antibodies of the present invention are therefore defined by the lack of cross-reactivity against unfixed mammalian tissue sections in vitro. This property is novel, unexpected and beneficial, and represents a special technical feature of the invention.

By way of example, exemplary antibodies as described herein, that exhibit low cross-reactivity to other tissue types, are CV07-250, CV38-183, CV07-209, or CV38-142. Further exemplary antibodies with this beneficial property are described in the examples below.

In some embodiments, the antibody or antibody fragment of the invention exhibits a strong affinity to the SARS-CoV-2 Spike protein target.

In some embodiments, Surface Plasmon Resonance (APR) measurements can quantitatively determine the affinity of an antibody or fragment to its target, for example by using the K_{D} values determined using SPR.

In some embodiments, the antibody or antibody fragment has an affinity, preferably determined using K_{D} values, preferably determined using SPR, or <500 nM, preferably <100 nM, <50 nM, <10 nM, more preferably <5 nM, <2 nM, <1 nM, more preferably <0.1 nM, or <0.01 nM.

By way of example, exemplary antibodies as described herein, that exhibit a good affinity to the SARS-CoV-2-Spike protein, are CV07-250, CV38-183, CV07-209, or CV38-142. Further exemplary antibodies with this beneficial property are described in the examples below.

In some embodiments, the antibody or antibody fragment of the invention exhibits beneficial properties with respect to binding and/or neutralizing multiple SARS-CoV-2 variants or mutants.

In one embodiment, the antibody or antibody fragment of the invention binds the Spike protein of and/or neutralizes the coronavirus SARS-CoV-1 and/or SARS-MERS.

In one embodiment, the antibody or antibody fragment of the invention binds the Spike protein of and/or neutralizes one or more, preferably multiple, coronavirus variants, preferably two or three variants, selected from the list consisting of the originally discovered Wuhan (or WT) virus, the B.1.1.7 variant and the B.1.351 variant.

In one embodiment, the antibody or antibody fragment of the invention binds the Spike protein of and/or neutralizes one or more, preferably multiple, coronavirus variants, preferably two or three or more variants, selected from the list consisting of the originally discovered Wuhan (or WT) virus, B.1.1.7, B.1.351, P.1, B.1.1.207, B.1.1.248, B.1.1.317, B.1.1.318, B.1.429, B.1.525,B.1.526, B.1.617, B.1.618, B.1.620 and P.3.

In one embodiment, the originally discovered Wuhan (or WT) SARS-CoV-2 virus is represented by the Munich isolate 984.

Preferred, non-limiting examples of the SARS-CoV-2 RBD mutants are according to SEQ ID NO 3 (the Wuhan (or WT) virus), SEQ ID NO 162 (the B.1.1.7 variant) and SEQ ID NO 164 (the B.1.351 variant).

In one embodiment, the antibody or antibody fragment of the invention binds the Spike protein of and/or neutralizes one or more, preferably multiple, coronavirus variants, but does not effectively inhibit an ACE2-Spike interaction.

In one embodiment, the antibody or antibody fragment of the invention binds the variant/mutated Spike protein of one or more variants, and effectively prevents virus infection of target cells. Such properties can be demonstrated using various assays available to a skilled person, for example, using a PRNT assay, as described herein, and optionally complemented using a SARS-CoV-2 ELISA assay to show binding, as described herein.

By way of example, exemplary antibodies as described herein, that exhibit beneficial properties with respect to binding and/or neutralizing multiple SARS-CoV-2 variants or mutants, are CV38-183, CV07-209, or CV38-142. Further exemplary antibodies with this beneficial property are described in the examples below. In a preferred embodiment, the antibody CV38-142 exhibits the unexpected and beneficial property of binding and/or neutralizing multiple SARS-CoV-2 variants or mutants.

A skilled person is aware that the SARS-CoV-2 variants presently known are not limiting and are continuing to be expanded upon further discovery and characterization of further variants. The ability of an antibody or fragment thereof of the present invention to bind multiple virus variants is beneficial and as such not expected, based on the antibodies already described in the prior art. It could not have been predicted based on common general knowledge that the antibodies of the invention, defined preferably by their amino acid sequences, would exhibit this property.

In some embodiments, promising antibody candidates are therefore defined by (1) Spike RBD binding, (2) ACE2 competition and (3) virus neutralization, and additionally by (4) non-binding to unfixed mammalian tissue. This combination of features represents a unique functional definition, derived from the selection method and characterization of the antibodies, which has, to the knowledge of the inventors, not been mentioned in the art.

In some embodiments, promising antibody candidates are defined by (1) Spike RBD binding, (2) virus neutralization, (3) non-binding to unfixed mammalian tissue, and additionally (4) binding and/or neutralizing multiple SARS-CoV-2 variants or mutant. This combination of features represents a unique functional definition, derived from the selection method and characterization of the antibodies, which has, to the knowledge of the inventors, not been mentioned in the art.

In some embodiments, the antibodies of the invention are characterized by binding to SARS-CoV-2-Spike-S1-RBD glycoprotein, and additionally by inhibiting the infection of epithelial cells (preferably kidney epithelial cells, such as VeroE6-cells) with SARS-CoV-2.

In some embodiments, the antibodies of the invention are characterized by binding to SARS-CoV-2-Spike-S1-RBD glycoprotein, and additionally by inhibiting the interaction between angiotensin-converting enzyme 2 (ACE2) or fragment thereof (preferably according to SEQ ID NO 7) with the SARS-CoV-2-Spike-S1-RBD.

In some embodiments, the antibodies of the invention are characterized by binding to SARS-CoV-2-Spike-S1-RBD glycoprotein, and additionally by inhibiting the interaction between angiotensin-converting enzyme 2 (ACE2) or fragment thereof (preferably according to SEQ ID NO 7) with the SARS-CoV-2-Spike-S1-RBD, and additionally by inhibiting the infection of epithelial cells (preferably kidney epithelial cells, such as VeroE6-cells) with SARS-CoV-2.

In some embodiments, the antibodies of the invention are characterized by binding to SARS-CoV-2-Spike-S1-RBD glycoprotein, and additionally by inhibiting the interaction between angiotensin-converting enzyme 2 (ACE2) or fragment thereof (preferably according to SEQ ID NO 7) with the SARS-CoV-2-Spike-S1-RBD, and additionally by inhibiting the infection of epithelial cells (preferably kidney epithelial cells, such as VeroE6-cells) with SARS-CoV-2, and additionally by not binding, or binding at negligible levels, unfixed mammalian tissue sections in vitro, said sections preferably selected from one or more of brain, heart, kidney and/or lung tissue sections.

In some embodiments, the antibodies of the invention are characterized by binding to SARS-CoV-2-Spike-S1-RBD glycoprotein, and additionally by inhibiting the infection of epithelial cells (preferably kidney epithelial cells, such as VeroE6-cells) with SARS-CoV-2, and additionally by not binding, or binding at negligible levels, unfixed mammalian tissue sections in vitro, said sections preferably selected from one or more of brain, heart, kidney and/or lung tissue sections, and additionally binding and/or neutralizing multiple SARS-CoV-2 variants or mutants.

The combinations of these properties could not have been predicted by a skilled person and therefore relate to non-obvious, special technical features of the invention.

In a further aspect, the invention relates to an antibody or antibody fragment as described herein for use in the treatment and/or prevention of a medical condition associated with a SARS Coronavirus.

In one embodiment, the medical condition associated with a SARS Coronavirus is COVID-19.

In one embodiment, the medical condition associated with a SARS Coronavirus is a SARS Coronavirus-associated respiratory disease.

The invention therefore relates to corresponding methods of treatment, comprising the administration of an antibody or antibody fragment as described herein to a subject in need thereof in order to treat and/or prevent a medical condition associated with a SARS Coronavirus.

The medical use of the antibodies described herein represents a solution to a medical problem, for which until the present time, no effective solutions have been proposed. The COVID-19 pandemic continues to spread and therapeutic options are desperately need by the medical community in order to reduce mortality and prevent the occurrence of severe symptoms and spread of the disease.

In some embodiments, the antibodies may be administered alone, or in combination with one or more other antibodies against CoV, preferably another antibody or fragment thereof from the present invention. In some embodiments, multiple antibodies of the invention are administered together, for example 2, 3, 4, 5 or more antibodies are combined to improve efficacy, i.e. by more effectively blocking the Spike-ACE2 interaction. In some embodiments, a cocktail of multiple antibodies is administered, comprising between 2 and 20, preferably between 2-5 antibodies of the present invention.

In some embodiments, an antibody or antibody fragment of the invention that disrupts or inhibits the ACE2-Spike interaction is administered with a different therapeutic antibody, that binds Spike protein, but does not inhibit the ACE2-Spike interaction. In some embodiments, an antibody or antibody fragment of the invention that does not inhibit the ACE2-Spike interaction is administered with a different therapeutic antibody, that binds Spike protein and disrupts or inhibits the ACE2-Spike interaction. In some embodiments, the antibody CV38-142 is administered in combination with another antibody that disrupts the ACE2-Spike interaction.

As is shown in the Examples below, CV38-142 binds an epitope of the Spike protein that is distinct from antibodies that inhibit the ACE2-Spike interaction. In some examples, CV38-142 showed a synergistic effect in a pseudovirus neutralization assay when administered in combination with another anti-spike antibody, COVA1-16, which interrupts the ACE2-Spike interaction. In this context, the second antibody COVA1-16 is considered an example of an anti-spike antibody, which interrupts the ACE2-Spike interaction, and may be replaced by other such antibodies, for example those described herein, such as CV07-250, CV38-183, or CV07-209. It is a unique and unexpected advantage that CV38-142 binds an epitope of SARS-CoV-2 spike protein that (a) appears conserved in multiple SARS-CoV-2 variants and thus allows binding to and neutralization of multiple SARS-CoV-2 variants, and (b) allows complementary binding to spike protein by another antibody that blocks the ACE2-spike interaction. This combination of advantageous properties is inherent in CV38-142 and could not have been derived from other antibodies, described prior to the present invention. The synergy between CV38-142 and other anti-spike antibodies, which interrupt the ACE2-Spike interaction, is also considered an unexpected advantage and is applicable to other antibody combinations comprising CV38-142 and other antibodies. In embodiments of the invention, the antibodies as described herein may be administered to various patient groups, for example therapeutically (e.g. to patients with SARS-related illnesses, including critically ill patients) or prophylactically (e.g. to patients who are at risk of having contracted a SARS Coronavirus infection, or to patients who are infected with a SARS Coronavirus but have not yet developed serious health issues).

In one embodiment, the antibody or antibody fragment described herein is administered to a patient with symptoms of an infectious disease.

In one embodiment, the antibody or antibody fragment described herein is administered to a patient with symptoms of a viral infection of the respiratory tract.

In one embodiment, the antibody or antibody fragment described herein is administered to a patient that is at risk of developing a severe acute respiratory syndrome (SARS).

In one embodiment, the antibody or antibody fragment described herein is administered to an asymptomatic patient that shows no specific symptoms of SARS.

In one embodiment, the antibody or antibody fragment described herein is administered to a patient that has or is at risk of developing a severe acute respiratory syndrome (SARS) and has a SARS Coronavirus infection.

In one embodiment, the antibody or antibody fragment described herein is administered to a patient that suffers from an infection, such as with a SARS Coronavirus (SARS-CoV). In preferred embodiments, the SARS Coronavirus is SARS-CoV-2.

In one embodiment, the antibody or antibody fragment described herein is administered to a patient that suffers from an infection with a SARS-CoV-2 coronavirus, selected from the list consisting of the originally discovered Wuhan (or WT) virus, the B.1.1.7 variant and the B.1.351 variant.

In one embodiment, the antibody or antibody fragment described herein is administered to a patient that suffers from an infection with a SARS-CoV-2 coronavirus, selected from the list consisting of the originally discovered Wuhan (or WT) virus, B.1.1.7, B.1.351, P.1, B.1.1.207, B.1.1.248, B.1.1.317, B.1.1.318, B.1.429, B.1.525, B.1.526, B.1.617, B.1.618, B.1.620 and P.3.

In a further aspect, the invention relates to an in vitro method for determining the presence or absence of SARS-CoV-2 viral protein in a sample.

In one embodiment, the method comprises contacting an antibody or antibody fragment as described herein with the sample to enable formation of an antibody-SARS-CoV-2 complex and subsequent detecting of said antibody-SARS-CoV-2 complex if present in said sample.

The diagnostic application of the antibodies described herein also represents a unique and beneficial method of determining the presence of Coronavirus particles (protein thereof) in a patient sample. The selection of SARS-CoV-2 specific antibodies, as described herein, therefore allows the development of an in vitro assay, such as an ELISA or lateral flow test for direct virus detection from patient swabs, or any bodily fluid with the suspected presence of virus.

In some embodiments, typically two SARS-CoV-2 specific monoclonal antibodies are used for this purpose: A first antibody (capture antibody) binds the virus from human samples to a microtiter plate, a second monoclonal SARS-CoV-2 selective antibody is used for detection of the bound virus and leads to a quantifiable color signal through an enzymatic reaction. The test principle for the detection of a molecule with two specific antibodies is well established (e.g. as in a pregnancy test).

Until now, direct virus detection has been carried out by amplifying the genetic virus material by means of a polymerase chain reaction (PCR). The procedure is dependent on complex equipment and takes several hours. The assay proposed here can (analogous to other lateral flow assays) provide a result after only a few minutes, can also be used in mobile applications (no laboratory required) and can be kept in very large quantities. As a rapid test, it enables the low threshold testing of large populations, e.g. of hospital staff before entering the ward, nursing staff in old people's homes or visitors to a (large) event. Such an assay can therefore be an essential building block in identifying patients with a Coronavirus infection.

In a further aspect of the invention, the antibodies may be defined by their antibody sequences.

The definition of an antibody may occur either in combination with, or independently of, the definition of the antibody by the functional features described herein. Furthermore, in some embodiments, the definition of the antibodies via their method of isolation may be combined with information on the antibody sequence, or the information on the antibody sequence used as the only definition of the inventive antibodies.

In one embodiment, the antibody or antibody fragment is defined by one or more complementary determining regions (CDRs) as described herein, preferably by 6 CDR regions, 3 from the variable region of the heavy chain, and 3 from the variable region of the light chain. The CDRs described herein may be used in the specific combination as discovered, or in any given combination for which the binding to the target protein is maintained.

In one embodiment, the antibody or antibody fragment comprises:
a heavy chain (variable VH) domain, said VH domain comprising complementary determining region (CDR) sequences of:
   a. H-CDR1 selected from a sequence with at least 70%, preferably at least 80% or at least 90%, or more preferably at least 95% sequence identity to SEQ ID NO 10, 18, 26, 34, 42, 50, 58, 66, 74, 82, 90, 98, 106, 114, 122, 130, 138, 146 or 154,
   b. H-CDR2 selected from a sequence with at least 70%, preferably at least 80% or at least 90%, or more preferably at least 95% sequence identity to SEQ ID NO 11, 19, 27, 35, 43, 51, 59, 67, 75, 83, 91, 99, 107, 115, 123, 131, 139, 147 or 155,
   c. H-CDR3 selected from a sequence with at least 70%, preferably at least 80% or at least 90%, or more preferably at least 95% sequence identity to SEQ ID NO 12, 20, 28, 36, 44, 52, 60, 68, 76, 84, 92, 100, 108, 116, 124, 132, 140, 148 or 156,
and a light chain variable (VL) domain, said VL domain comprising complementary determining region (CDR) sequences of:
   d. L-CDR1 selected from a sequence with at least 70%, preferably at least 80% or at least 90%, or more preferably at least 95% sequence identity to SEQ ID NO 13, 21, 29, 37, 45, 53, 61, 69, 77, 85, 93, 101, 109, 117, 125, 133, 141, 149 or 157,
   e. L-CDR2 selected from a sequence with at least 70%, preferably at least 80% or at least 90%, or more preferably at least 95% sequence identity to SEQ ID NO 14, 22, 30, 38, 46, 54, 62, 70, 78, 86, 94, 102, 110, 118, 126, 134, 142, 150 or 158,
   f. L-CDR3 selected from a sequence with at least 70%, preferably at least 80% or at least 90%, or more preferably at least 95% sequence identity to SEQ ID NO 15, 23, 31, 39, 47, 55, 63, 71, 79, 87, 95, 103, 111, 119, 127, 135, 143, 151 or 159.

In one embodiment, the antibody or antibody fragment comprises:
a heavy chain (variable VH) domain, said VH domain comprising complementary determining region (CDR) sequences of:
   a. H-CDR1 selected from SEQ ID NO 10, 18, 26, 34, 42, 50, 58, 66, 74, 82, 90, 98, 106, 114, 122, 130, 138, 146 or 154,
   b. H-CDR2 selected from SEQ ID NO 11, 19, 27, 35, 43, 51, 59, 67, 75, 83, 91, 99, 107, 115, 123, 131, 139, 147 or 155,
   c. H-CDR3 selected from SEQ ID NO 12, 20, 28, 36, 44, 52, 60, 68, 76, 84, 92, 100, 108, 116, 124, 132, 140, 148 or 156,
and a light chain variable (VL) domain, said VL domain comprising complementary determining region (CDR) sequences of:
   d. L-CDR1 selected from SEQ ID NO 13, 21, 29, 37, 45, 53, 61, 69, 77, 85, 93, 101, 109, 117, 125, 133, 141, 149 or 157,
   e. L-CDR2 selected from SEQ ID NO 14, 22, 30, 38, 46, 54, 62, 70, 78, 86, 94, 102, 110, 118, 126, 134, 142, 150 or 158,
   f. L-CDR3 selected from SEQ ID NO 15, 23, 31, 39, 47, 55, 63, 71, 79, 87, 95, 103, 111, 119, 127, 135, 143, 151 or 159.

In one embodiment, the antibody or antibody fragment comprises 6 CDRs according to:
SEQ ID NO 10-15, 18-23, 26-31, 34-39, 42-47, 50-55, 58-63, 66-71, 74-79, 82-87, 90-95, 98-103, 106-111, 114-119, 122-127, 130-135, 138-143, 146-151 or 154-159.

With respect to the CDR sequences and sequence variation with any given sequence identity to the specific sequence, a skilled person is capable of determining antibody binding and can obtain, without undue effort, a set of CDR sequences with the given sequence identity that maintain the binding properties of the antibodies disclosed herein.

Should the sequence be of a length, that cannot plausibly have sequence variation that would lead to an e.g. 90% sequence identity, a skilled person is also capable of determining such sequences and adjusting or excluding the sequences appropriately. Considering the detailed information presented in the examples regarding the desired binding characteristics of the antibodies, any given antibody with sufficient sequence identity can be determined without undue effort.

Regarding the L-CDR2 sequences, these comprise or consist of, in some embodiments, three amino acids. In some embodiments, amino acid sequence variation is possible, such that e.g. 1 of the 3 amino acids is changed from the original sequence and binding is maintained. Such variants, with ⅔ amino acid identities to the original sequences, especially those with conserved substitutions, are encompassed in the scope of the invention. The specific L-CDR2 sequences are provided below in Table 1.

Regarding the L-CDR2 sequences, the sequences are as follows: SEQ ID NO 14 (GVR), 22 (EVS), 30 (EVS), 38 (GAS), 46 (DAS), 54 (DAS), 62 (EGS), 70 (EGS), 78 (DAS), 86 (ANS), 94 (EVS), 102 (ENN), 110 (AAS), 118 (AAS), 126 (AAS), 134 (GAS), 142 (AAS), 150 (AAS) or 158 (EVS).

In one embodiment, the antibody or antibody fragment of the invention comprises:
a heavy chain variable (VH) domain, said VH domain comprising a sequence of at least 70%, preferably at least 80% or at least 90%, or more preferably at least 95% sequence identity to one of SEQ ID NO 16, 24, 32, 40, 48, 56, 64, 72, 80, 88, 96, 104, 112, 120, 128, 136, 144, 152 or 160, and
a light chain variable (VL) domain, said VL domain comprising a sequence of at least 70%, preferably at least 80% or at least 90%, or more preferably at least 95% sequence identity to one of SEQ ID NO 17, 25, 33, 41, 49, 57, 65, 73, 81, 89, 97, 105, 113, 121, 129, 137, 145, 153 or 161.

In one embodiment, the VH and VL combinations are as described in the antibodies as originally isolated.

In one embodiment, the antibody or antibody fragment comprises VH and VL domains that comprise the sequences according to SEQ ID NO 16 and 17, 24 and 25, 32 and 33, 40 and 41, 48 and 49, 56 and 57, 64 and 65, 72 and 73, 80 and 81, 88 and 89, 96 and 97, 104 and 105, 112 and 113, 120 and 121, 128 and 129, 136 and 137, 144 and 145, 152 and 153, and 160 and 161, respectively.

In one embodiment, the antibody or antibody fragment comprises VH and VL domains with the specific CDR sequences as described in the antibodies as isolated, and additionally are characterized by a framework sequence with sequence similarity as described herein to the specific VH and VL sequences as isolated.

For example, the CDR sequences are those as specified in the isolated antibodies, and outside the specified CDR sequences, the antibodies comprise adjacent framework sequences with:
an at least 70%, preferably at least 80% or at least 90%, or more preferably at least 95% or complete sequence identity to the relevant non-CDR portion of one of SEQ ID NO 16, 24, 32, 40, 48, 56, 64, 72, 80, 88, 96, 104, 112, 120, 128, 136, 144, 152 or 160 as a VH sequence, and
an at least 70%, preferably at least 80% or at least 90%, or more preferably at least 95% or complete sequence identity to the relevant non-CDR portion of one of SEQ ID NO 17, 25, 33, 41, 49, 57, 65, 73, 81, 89, 97, 105, 113, 121, 129, 137, 145, 153 or 161 as a VL sequence.

In embodiments of the invention, the amino acid sequence variation used to obtain said antibodies may occur in either the CDR regions of the original antibody or in the framework regions, wherein the framework region is to be understood as a region in the variable domain of a protein which belongs to the immunoglobulin superfamily, and which is less “variable” than the CDRs.

In preferred embodiments, any sequence variant that maintains any one or more of the binding properties disclosed herein is encompassed by the invention.

It was entirely surprising that the particular antibodies provided herein, preferably defined by the CDR regions of the VL and VH regions involved in binding, or by the VH and VL sequences disclosed herein, exhibit the properties as demonstrated in the examples, and show binding characteristics that enable the desired therapeutic and/or diagnostic effect.

In general, any change to a CDR region made may also be considered as a feature of a CDR sequence when considered independently of the framework sequence as a whole. Such modified CDR sequences may be considered defining features of the present invention, either within or independent of their context in the entire framework region described herein. For example, the CDR sequences identified above may be considered a defining feature of the invention independently of the surrounding framework sequence. In some embodiments, sequence variation of VH and VL antibody sequences by way of percentage sequence identity can be employed, combined with specific CDR sequences.

All possible combinations of potential modifications for any given potentially variant residue proposed herein are encompassed by the present invention. By combining one or more of these substitutions, variants may be generated that exhibit the desired binding properties of the antibody originally isolated. The antibodies or parts thereof described herein also encompass a sequence with at least 70%, 75%, 80%, 85%, preferably 90%, or 95% sequence identity to those sequences disclosed explicitly.

A further aspect of the invention relates to a nucleic acid molecule comprising a nucleotide sequence that encodes an antibody or antibody fragment as described herein.

In one embodiment, the invention relates to a preferably isolated nucleic acid molecule selected from the group consisting of:
a) a nucleic acid molecule comprising a nucleotide sequence
   which encodes an isolated antibody or antibody fragment as described herein,
   which encodes an amino acid sequence selected from the group consisting of those sequences according to:
      a. as VH sequences SEQ ID NO 16, 24, 32, 40, 48, 56, 64, 72, 80, 88, 96, 104, 112, 120, 128, 136, 144, 152 or 160, and/or
      b. as VL sequences SEQ ID NO 17, 25, 33, 41, 49, 57, 65, 73, 81, 89, 97, 105, 113, 121, 129, 137, 145, 153 or 161,
   b) a nucleic acid molecule which is complementary to a nucleotide sequence in accordance with a);
   c) a nucleic acid molecule comprising a nucleotide sequence having sufficient sequence identity to be functionally analogous/equivalent to a nucleotide sequence according to a) or b),
   d) a nucleic acid molecule which, as a consequence of the genetic code, is degenerated into a nucleotide sequence according to a) through c); and
   e) a nucleic acid molecule according to a nucleotide sequence of a) through d) which is modified by deletions, additions, substitutions, translocations, inversions and/or insertions and functionally analogous/equivalent to a nucleotide sequence according to a) through d).

A further aspect of the invention relates to a host cell, such as a bacterial cell or mammalian cell, capable of producing an antibody or antibody fragment and/or comprising a nucleic acid molecule as described herein.

A further aspect of the invention relates to a pharmaceutical composition comprising the isolated antibody or antibody fragment or a nucleic acid molecule or a host cell as described herein, with a pharmaceutically acceptable carrier.

Further examples of pharmaceutical compositions and host cells are described in detail below.

### Embodiments to CV38-138

In one preferred aspect of the invention, the antibody or antibody fragment is or is derived from the antibody CV38-138.

The heavy and light chain variable amino acid sequences of the antibody CV38-138 (VH and VL) were isolated from convalescent COVID-19 patients.

In one embodiment, the antibody or antibody fragment according to CV38-138 inhibits the interaction between angiotensin-converting enzyme 2 (ACE2) or fragment thereof (preferably according to SEQ ID NO 7) with the SARS-CoV-2-Spike-S1-RBD.

In one embodiment, the inhibition of interaction between a soluble fragment of ACE2 (preferably according to SEQ ID NO 7) and SARS-CoV-2-Spike-S1-RBD (preferably according to SEQ ID NO 3) obtained by CV38-138 is at least 30%, preferably at least 60%, in an in vitro competition assay in which HEK-SARS-CoV-2-Spike-S1-RBD glycoprotein is immobilized on a solid phase and incubated with the antibody or antibody fragment and subsequently with ACE2.

In one embodiment, the antibody or antibody fragment according to CV38-138 inhibits the infection of epithelial cells (preferably kidney epithelial cells, such as VeroE6-cells) with SARS-CoV-2.

In one embodiment, the antibody or antibody fragment according to CV38-138 has an IC50 of <10 ng/mL, preferably about 3.7 ng/mL, in a plaque reduction neutralization test (PRNT).

In one embodiment, the antibody or antibody fragment according to CV38-138 does not bind, or binds at negligible levels, unfixed mammalian tissue sections in vitro, said sections preferably selected from one or more of brain, heart, kidney and/or lung tissue sections.

In one embodiment, the antibody or antibody fragment according to CV38-138 comprises:
a heavy chain (variable VH) domain, said VH domain comprising complementary determining region (CDR) sequences of:
a. H-CDR1 according to SEQ ID NO 82,
b. H-CDR2 according to SEQ ID NO 83,
c. H-CDR3 according to SEQ ID NO 84,
and a light chain variable (VL) domain, said VL domain comprising complementary determining region (CDR) sequences of:
a. L-CDR1 according to SEQ ID NO 85,
b. L-CDR2 according to SEQ ID NO 86,
c. L-CDR3 according to SEQ ID NO 87.

In one embodiment, the antibody or antibody fragment according to CV38-138 comprises:

a heavy chain variable (VH) domain, said VH domain comprising a sequence of at least 70%, 80%, 85%, 90%, at least 95% or complete identity to SEQ ID NO 88, and a light chain variable (VL) domain, said VL domain comprising a sequence of at least 70%, 80%, 85%, 90%, at least 95% or complete identity to SEQ ID NO 89.

Further embodiments relate to a nucleic acid molecule comprising a nucleotide sequence that encodes CV38-138 or fragment thereof, a host cell, such as a bacterial cell or mammalian cell, capable of producing CV38-138 or fragment thereof, a pharmaceutical composition comprising CV38-138 or fragment thereof, a method for the treatment and/or prevention of a medical condition associated with a SARS Coronavirus comprising administering CV38-138 or fragment thereof, and an in vitro method comprising using CV38-138 or fragment thereof.

### Embodiments to CV07-209

In one preferred aspect of the invention, the antibody or antibody fragment is or is derived from the antibody CV07-209.

The heavy and light chain variable amino acid sequences of the antibody CV07-209 (VH and VL) were isolated from convalescent COVID-19 patients.

In one embodiment, the antibody or antibody fragment according to CV07-209 binds the Spike protein of and/or neutralizes one or more, preferably multiple, coronavirus variants, preferably two or three or more variants, selected from the list consisting of the originally discovered Wuhan (or WT) virus, B.1.1.7, B.1.351, P.1, B.1.1.207, B.1.1.248, B.1.1.317, B.1.1.318, B.1.429, B.1.525, B.1.526, B.1.617, B.1.618, B.1.620 and P.3.

In one embodiment, the antibody or antibody fragment according to CV07-209 inhibits the interaction between angiotensin-converting enzyme 2 (ACE2) or fragment thereof (preferably according to SEQ ID NO 7) with the SARS-CoV-2-Spike-S1-RBD.

In one embodiment, the inhibition of interaction between a soluble fragment of ACE2 (preferably according to SEQ ID NO 7) and SARS-CoV-2-Spike-S1-RBD (preferably according to SEQ ID NO 3) obtained by CV07-209 is at least 10%, preferably at least 70%, in an in vitro competition assay in which HEK-SARS-CoV-2-Spike-S1-RBD glycoprotein is immobilized on a solid phase and incubated with the antibody or antibody fragment and subsequently with ACE2.

In one embodiment, the antibody or antibody fragment according to CV07-209 inhibits the infection of epithelial cells (preferably kidney epithelial cells, such as VeroE6-cells) with SARS-CoV-2.

In one embodiment, the antibody or antibody fragment according to CV07-209 has an IC50 of <10 ng/mL, preferably about 3.1 ng/mL, in a plaque reduction neutralization test (PRNT).

In one embodiment, the antibody or antibody fragment according to CV07-209 does not bind, or binds at negligible levels, unfixed mammalian tissue sections in vitro, said sections preferably selected from one or more of brain, heart, kidney and/or lung tissue sections.

In one embodiment, the antibody or antibody fragment according to CV07-209 comprises:
a heavy chain (variable VH) domain, said VH domain comprising complementary determining region (CDR) sequences of:
a. H-CDR1 according to SEQ ID NO 50,
b. H-CDR2 according to SEQ ID NO 51,
c. H-CDR3 according to SEQ ID NO 52,
and a light chain variable (VL) domain, said VL domain comprising complementary determining region (CDR) sequences of:
a. L-CDR1 according to SEQ ID NO 53,
b. L-CDR2 according to SEQ ID NO 54,
c. L-CDR3 according to SEQ ID NO 55.

In one embodiment, the antibody or antibody fragment according to CV07-209 comprises:
a heavy chain variable (VH) domain, said VH domain comprising a sequence of at least 70%, 80%, 85%, 90%, at least 95% or complete identity to SEQ ID NO 56, and a light chain variable (VL) domain, said VL domain comprising a sequence of at least 70%, 80%, 85%, 90%, at least 95% or complete identity to SEQ ID NO 57.

Further embodiments relate to a nucleic acid molecule comprising a nucleotide sequence that encodes CV07-209 or fragment thereof, a host cell, such as a bacterial cell or mammalian cell, capable of producing CV07-209 or fragment thereof, a pharmaceutical composition comprising CV07-209 or fragment thereof, a method for the treatment and/or prevention of a medical condition associated with a SARS Coronavirus comprising administering CV07-209 or fragment thereof, and an in vitro method comprising using CV07-209 or fragment thereof.

### Embodiments to CV38-142

In one preferred aspect of the invention, the antibody or antibody fragment is or is derived from the antibody CV38-142.

The heavy and light chain variable amino acid sequences of the antibody CV38-142 (VH and VL) were isolated from convalescent COVID-19 patients.

In one embodiment, the antibody or antibody fragment according to CV38-142 binds the Spike protein of and/or neutralizes one or more, preferably multiple, coronavirus variants, preferably two or three or more variants, selected from the list consisting of the originally discovered Wuhan (or WT) virus, B.1.1.7, B.1.351, P.1, B.1.1.207, B.1.1.248, B.1.1.317, B.1.1.318, B.1.429, B.1.525, B.1.526, B.1.617, B.1.618, B.1.620 and P.3.

In one embodiment, the antibody or antibody fragment according to CV38-142 only weakly inhibits, or does not effectively inhibit, the interaction between angiotensin-converting enzyme 2 (ACE2) or fragment thereof (preferably according to SEQ ID NO 7) with the SARS-CoV-2-Spike-S1-RBD.

In one embodiment, the inhibition of interaction between a soluble fragment of ACE2 (preferably according to SEQ ID NO 7) and SARS-CoV-2-Spike-S1-RBD (preferably according to SEQ ID NO 3) obtained by CV38-142 is at least 10%, preferably at least 20%, in an in vitro competition assay in which HEK-SARS-CoV-2-Spike-S1-RBD glycoprotein is immobilized on a solid phase and incubated with the antibody or antibody fragment and subsequently with ACE2.

In one embodiment, the antibody or antibody fragment according to CV38-142 inhibits the infection of epithelial cells (preferably kidney epithelial cells, such as VeroE6-cells) with SARS-CoV-2.

In one embodiment, the antibody or antibody fragment according to CV38-142 has an IC50 of <30 ng/mL, preferably about 23.2 ng/mL, in a plaque reduction neutralization test (PRNT).

In one embodiment, the antibody or antibody fragment according to CV38-142 does not bind, or binds at negligible levels, unfixed mammalian tissue sections in vitro, said sections preferably selected from one or more of brain, heart, kidney and/or lung tissue sections.

In one embodiment, the antibody or antibody fragment according to CV38-142 comprises:
a heavy chain (variable VH) domain, said VH domain comprising complementary determining region (CDR) sequences of:
a. H-CDR1 according to SEQ ID NO 114,
b. H-CDR2 according to SEQ ID NO 115,
c. H-CDR3 according to SEQ ID NO 116,
and a light chain variable (VL) domain, said VL domain comprising complementary determining region (CDR) sequences of:
a. L-CDR1 according to SEQ ID NO 117,
b. L-CDR2 according to SEQ ID NO 118,
c. L-CDR3 according to SEQ ID NO 119.

In one embodiment, the antibody or antibody fragment according to CV38-142 comprises:
a heavy chain variable (VH) domain, said VH domain comprising a sequence of at least 70%, 80%, 85%, 90%, at least 95% or complete identity to SEQ ID NO 120, and a light chain variable (VL) domain, said VL domain comprising a sequence of at least 70%, 80%, 85%, 90%, at least 95% or complete identity to SEQ ID NO 121.

Further embodiments relate to a nucleic acid molecule comprising a nucleotide sequence that encodes CV38-142 or fragment thereof, a host cell, such as a bacterial cell or mammalian cell, capable of producing CV38-142 or fragment thereof, a pharmaceutical composition comprising CV38-142 or fragment thereof, a method for the treatment and/or prevention of a medical condition associated with a SARS Coronavirus comprising administering CV38-142 or fragment thereof, and an in vitro method comprising using CV38-142 or fragment thereof.

In some embodiments, an antibody fragment according to a particular named antibody is a polypeptide comprising the CDR and/or VH and/or VL sequences of the named antibody. In preferred embodiments, the fragment maintains in essence the binding properties, such as specificity and/or affinity, of the full antibody.

Features of the invention relating to the antibodies, methods of their preparation or use, including medical use, are considered disclosed in relation to all aspects of the invention. Features relating to the antibodies may be used to described aspects of their use, and vice versa.

### DETAILED DESCRIPTION OF THE INVENTION

The present invention relates to a human recombinant monoclonal antibody or antibody fragment that binds the receptor binding domain (RBD) of the S1 subunit (S1) of a recombinant Spike glycoprotein of SARS-CoV-2 expressed from human embryonic kidney (HEK293) cells (HEK-SARS-CoV-2-Spike-S1-RBD glycoprotein), wherein the heavy and light chain variable amino acid sequences of the antibody (VH and VL) were isolated from convalescent COVID-19 patients. Various related aspects and embodiments are encompassed by the invention.

As used herein, the term “receptor binding domain (RBD) of the S1 subunit (S1) of a recombinant Spike glycoprotein of SARS-CoV-2 expressed from human embryonic kidney (HEK293) cells” (abbrev. HEK-SARS-CoV-2-Spike-S1-RBD glycoprotein) relates to the target protein used to isolate the human antibodies of the present invention. In some embodiments, the antibodies of the present invention may therefore be CoV-2 specific, in other words, they may bind the Spike protein of the CoV-2 and not of other viruses. In some embodiments, the antibodies may bind the Spike protein of any given SARS Coronavirus. In some embodiments, the antibodies may bind the Spike protein of a Coronavirus that evolves or mutates from CoV-2 to form a new virus, albeit with sufficient similarity to be bound by the antibodies of the invention. In some embodiments, the antibodies bind a SARS Coronavirus (also referred to herein as a “Coronavirus”, or as “the virus”), more preferably to SARS-CoV-2. The antibodies of the invention are therefore considered to bind the RBD of the Spike protein of SARS-CoV-2 as it exists in subjects, i.e. in the form it takes in human subjects before, during or after infection of a host cell.

### Antibodies

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

The antibodies of the invention are intended to bind against viral targets, in particular SARS-Coronavirus protein targets, in particular the receptor binding domain (RBD) of the S1 subunit (S1) of a recombinant Spike glycoprotein of SARS-CoV-2 expressed from human embryonic kidney (HEK293) cells (HEK-SARS-CoV-2-Spike-S1-RBD glycoprotein).

“Specific binding” is to be understood as via one skilled in the art, whereby the skilled person is clearly aware of various experimental procedures that can be used to test binding and binding specificity. Some cross-reaction or background binding may be inevitable in many protein-protein interactions; this is not to detract from the “specificity” of the binding between antibody and epitope. The term “directed against” is also applicable when considering the term “specificity” in understanding the interaction between antibody and epitope.

Antibodies of the invention include, but are not limited to polyclonal, monoclonal, bispecific, human or chimeric antibodies, single variable fragments (ssFv), single domain antibodies (such as VHH fragments from nanobodies), single chain fragments (scFv), Fab fragments, F(ab′)₂ fragments, fragments produced by a Fab expression library, anti-idiotypic antibodies and epitope-binding fragments or combinations thereof of any of the above, provided that they retain the original binding properties. Also mini-antibodies and multivalent antibodies such as diabodies, triabodies, tetravalent antibodies and peptabodies can be used in a method of the invention. The immunoglobulin molecules of the invention can be of any class (i.e. IgG, IgE, IgM, IgD and IgA) or subclass of immunoglobulin molecules. Thus, the term antibody, as used herein, also includes antibodies and antibody fragments either produced by the modification of whole antibodies or synthesized de novo using recombinant DNA methodologies.

The present invention further relates to the use of the antibodies, or fragments thereof, as described herein, for example the variable regions, in recognition molecules or affinity reagents that are suitable for selective binding to a target. The affinity reagent, antibody or fragment thereof according to the invention may be PEGylated, whereby PEGylation refers to covalent attachment of polyethylene glycol (PEG) polymer chains to the inventive antibody. PEGylation may be routinely achieved by incubation of a reactive derivative of PEG with the target molecule. PEGylation to the antibody can potentially mask the agent from the host's immune system, leading to reduced immunogenicity and antigenicity or increase the hydrodynamic size of the agent which may prolong its circulatory time by reducing renal clearance.

A variable region of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Iazikani et al. (1997) J. Molec. Biol. 273:927-948). As used herein, a CDR may refer to CDRs defined by either approach or by a combination of both approaches.

In some embodiments, the invention provides an antibody, which comprises at least one CDR, at least two, at least three, or more CDRs that are substantially identical to at least one CDR, at least two, at least three, or more CDRs of the antibody of the invention. Other embodiments include antibodies which have at least two, three, four, five, or six CDR(s) that are substantially identical to at least two, three, four, five or six CDRs of the antibodies of the invention or derived from the antibodies of the invention. In some embodiments, the at least one, two, three, four, five, or six CDR(s) are at least about 85%, 86%, 87%, 88%, 89%, 90%, 95%, 96%, 97%, 98%, or 99% identical to at least one, two or three CDRs of the antibody of the invention. It is understood that, for purposes of this invention, binding specificity and/or overall activity is generally retained, although the extent of activity may vary compared to said antibody (may be greater or lesser).

The half-life and cytotoxic potential of an antibody are dependent primarily on the interaction of the Fc-domain with different Fc-gamma-receptors. In the case of the antibody half-life, the neonatal Fc receptor (FcRn) plays a major role. This receptor is expressed on several cell types and tissues such as monocytes and vascular endothelia cells that are able to take up serum proteins into their recycling endosomes. In the endosomes, the pH is decreased to approximately 6 and under these conditions the antibodies are able to bind to FcRn. This interaction protects the antibodies from degradation until they are again released into the blood where the physiological pH disrupts the binding to the receptor (Roopenian and Akilesh (2007) Nat Rev Immunol 7:715-725). The higher the affinity of the antibody to the FcRn at pH 6, the greater the half-life of that antibody. Fc-fragment mutations known to stabilize this interaction are summarised in Presta (2008, Curr Opin Immunol 20:460-470).

### Antiviral Antibodies

Therapeutic anti-viral antibodies can act through several mechanisms upon binding to their target.

In some embodiments, mAb therapy is a form of passive immunotherapy that is intended to blunt a viral infection via direct and rapid targeting of the infectious agent rather than via the triggering of a long-term immune response against it. This therapeutic approach contrasts with vaccine approaches that aim to stimulate the endogenous immune response of the host, in order to provide sustained protective immunity.

mAbs can diminish viral dissemination by direct action involving both their antigen-binding activity and the effector functions borne by their Fc fragment. In some embodiments, the antiviral mAbs described herein have therapeutic potential and can be selected initially for their ability to neutralize virions via the recognition of a viral antigens essential for receptor binding and/or entry into host cells. In the context of the present invention, the viral antigen targeting by the inventive mAbs is the Spike protein of SARS-CoV-2. Furthermore, direct recognition has also been shown to inhibit cell-cell transmission of virions in certain settings.

In some embodiments, the antibodies of the present invention are immunoglobulin Gs (IgGs), that is, antibodies efficiently recognized by both the complement and the Fcγ receptors (FcγRs) borne by many cells of the immune system. In addition to complement-mediated inactivation of viral particles, and/or their phagocytosis, this also permits complement-dependent cytotoxicity (CDC), antibody-dependent cellular phagocytosis (ADCP), and antibody-dependent cell-mediated cytotoxicity (ADCC) to eliminate virus-infected cells potentially displaying the target viral antigen at their surface.

This, for instance, is the case of mAbs targeting the envelope glycoprotein (Env) of HIV (and other lenti-/retroviruses) that is exposed at the surface of virus-producing cells. Finally, Fc-FcγR interactions can also directly impact viral propagation via a mechanism called antibody-dependent, cell-mediated virus inhibition (ADCVI).

Thus, during immunotherapy mAbs can impact viral propagation via a variety of direct mechanisms.

With regard to cell-depletion there are two major effector mechanisms known. The first is the complement-dependent cytotoxicity (CDC) towards the target cell. There are three different pathways known. However, in the case of antibodies the important pathway for CDC is the classical pathway which is initiated through the binding of C1q to the constant region of IgG or IgM (Wang and Weiner (2008) Expert Opin Biol Ther 8:759-768).

The second mechanism is called antibody-dependent cellular cytotoxicity (ADCC). This effector function is characterized by the recruitment of immune cells which express Fc-receptors for the respective isotype of the antibody. ADCC is largely mediated by activating Fc-gamma receptors (FcγR) which are able to bind to IgG molecules either alone or as immune complexes. Mice exhibit three (FcγRI, FcγRIII and FcγRIV) and humans five (FcγRI, FcγRIIA, FcγRIIC, FcγRIIIA and FcγRIIIB) activating Fcγ-receptors. These receptors are expressed on innate immune cells like granulocytes, monocytes, macrophages, dendritic cells and natural killer cells and therefore link the innate with the adaptive immune system. Depending on the cell type there are several modes of action of FcgR-bearing cells upon recognition of an antibody-marked target. Granulocytes generally release vasoactive and cytotoxic substances or chemoattractants but are also capable of phagocytosis. Monocytes and macrophages respond with phagocytosis, oxidative burst, cytotoxicity or the release of pro-inflammatory cytokines whereas Natural killer cells release granzymes and perforin and can also trigger cell death through the interaction with FAS on the target cell and their Fas ligand (Nimmerjahn and Ravetch (2008) Nat Rev Immunol 8:34-47; Wang and Weiner (2008) Expert Opin Biol Ther 8:759-768; Chavez-Galan et al. (2009) Cell Mol Immunol 6:15-25).

### Sequence Variation

Sequence variants of the claimed nucleic acids, proteins and antibodies, for example defined by the claimed % sequence identity, that maintain the said properties of the invention are also included in the scope of the invention. Such variants, which show alternative sequences, but maintain essentially the same binding properties, such as target specificity, as the specific sequences provided are known as functional analogues, or as functionally analogous. Sequence identity relates to the percentage of identical nucleotides or amino acids when carrying out a sequence alignment.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein, preferably without significantly altering the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but FR alterations are also contemplated. If such substitutions result in a change in biological activity, then more substantial changes, denominated “exemplary substitutions” in the table immediately below, or as further described below in reference to amino acid classes, may be introduced and the products screened.

### Potential Amino Acid Substitutions

| | Preferred | |
|---|---|---|
| Original | conservative | Examples of exemplary |
| residue | substitutions | substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

### Antibody Production

The antibodies of the present invention may be produced by transfection of a host cell with an expression vector comprising the coding sequence for the antibody of the invention. An expression vector or recombinant plasmid is produced by placing these coding sequences for the antibody in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antibody light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The antibody which includes the association of both the recombinant heavy chain and/or light chain is screened from culture by appropriate assay, such as ELISA or RIA. Similar conventional techniques may be employed to construct other antibodies.

Suitable vectors for the cloning and subcloning steps employed in the methods and construction of the compositions of this invention may be selected by one of skill in the art. For example, the conventional pUC series of cloning vectors may be used. One vector, pUC19, is commercially available. The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antibodies of the present invention. Host cells useful for the cloning and other manipulations of these cloning vectors are also conventional.

Suitable host cells or cell lines for the expression of the antibodies of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other prokaryotic or eukaryotic cell lines may be employed. The selection of suitable mammalian host cells and methods for transformation, culture, amplification, screening and product production and purification are known in the art.

In accordance with the present invention there is provided a method of producing an anti-viral-antibody of the present invention which binds to and neutralises the activity of the virus which method comprises the steps of; providing a first vector encoding a heavy chain of the antibody; providing a second vector encoding a light chain of the antibody; transforming a mammalian host cell (e.g. CHO) with said first and second vectors; culturing the host cell of step (c) under conditions conducive to the secretion of the antibody from said host cell into said culture media; recovering the secreted antibody of step (d). Once expressed, the antibody can be assessed for the desired binding properties using methods as described herein.

### Pharmaceutical Compositions

In some embodiments, the invention relates to a pharmaceutical composition comprising the antibody or fragment thereof of the invention together with a pharmaceutically acceptable carrier. A pharmaceutically acceptable carrier in the sense of the present invention may be any non-toxic material that does not significantly interfere in a detrimental sense with the effectiveness of the biological activity of the antibodies of the present invention.

Evidently, the characteristics of the carrier will depend on the route of administration. Such a composition may contain, in addition to the active substance and carrier, diluents, fillers, salts, buffers, stabilizers, solubilizers, and other materials well known in the art. Formulation of pharmaceutically-acceptable excipients and carrier solutions is well-known to those of skill in the art, as is the development of suitable dosing and treatment regimens for using the particular compositions described herein in a variety of treatment regimens, including, without limitation, oral, parenteral, intravenous, sub-cutaneous, intranasal, inhalation, and intra-muscular administration.

The medicament, otherwise known as a pharmaceutical composition, containing the active ingredient (antibody or antibody fragment) may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets.

These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated. The present invention also refers to a pharmaceutical composition for topical application, oral ingestion, inhalation, or cutaneous, subcutaneous, or intravenous injection. A skilled person is aware of the carriers and additives required for particular application forms.

The medicament, otherwise known as a pharmaceutical composition, containing the active ingredient (antibody or antibody fragment) may be in a form suitable for injection. Modes of administration involving injection comprise, without limitation, Subcutaneous (under the skin), Intramuscular (in a muscle), Intravenous (in a vein) or Intrathecal (around the spinal cord). Antibody therapies are typically administered using intravenous administration, which is a preferred mode of administration of the present invention.

Excipients, as an example of pharmaceutically acceptable carrier, for liquid formulations intended for injection are known in the art and can be selected appropriately by a skilled person. Excipients have been used to increase the stability of a wide range of protein and peptide-based formulations by reducing protein dynamics and motion, increasing the conformational stability of mAbs especially at high concentrations and inhibiting interface-dependent aggregation. Excipients usually inhibit aggregation and protects the protein by adsorbing to the air-liquid interface; for example, the use of surfactants (e.g., polysorbate 20 and 80), carbohydrates (e.g., cyclodextrin derivatives) and amino acids (e.g., arginine and histidine) can help prevent aggregation by this mechanism. Cyclodextrin has been reported to stabilize commercially available antibody-based drugs in a hydrogel formulation. Some of the generally recognized as safe (GRAS) excipients include pluronic F68, trehalose, glycine and amino acids such as arginine, glycine, glutamate and histidine, which are found in a number of commercial protein therapeutic products. By way of example, bevacizumab, 25 mg/mL, contains trehalose dehydrate, sodium phosphate and polysorbate 20. Excipients in subcutaneous trastuzumab, 600 mg, are rHuPH20, histidine hydrochloride, histidine, trehalose dehydrate, polysorbate 20, methionine and water for injection.

When a therapeutically effective amount of the active substance (antibody or antibody fragment) of the invention is administered by intravenous, cutaneous or subcutaneous injection, the active substance may be in the form of a solution, preferably a pyrogen-free, parenterally acceptable aqueous solution.

The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to the active substance, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The invention also relates to administration of a therapeutically relevant amount of antibody as described herein in the treatment of a subject who has the medical disorders as disclosed herein. As used herein, the term “therapeutically effective amount” means the total amount of each active component of the pharmaceutical composition or method that is sufficient to show a meaningful patient benefit. The amount of active substance in the pharmaceutical composition of the present invention will depend upon the nature and severity of the condition being treated, and on the nature of prior treatments which the patient has undergone. Larger doses may be administered until the optimal therapeutic effect is obtained for the patient, and at that point the dosage is not increased further.

The dose of the antibody administered evidently depends on numerous factors well-known in the art such as, e.g., the chemical nature and pharmaceutical formulation of the antibody, and of body weight, body surface, age and sex of the patient, as well as the time and route of administration. For an adult, the dose may exemplarily be between 0.001 μg and 1 g per day, preferably between 0.1 μg and 100 mg per day, more preferably between 1 μg and 100 mg per day, even more preferably between 5 μg and 10 mg per day. In a continuous infusion, the dose may exemplarily be between 0.01 μg and 100 mg, preferably between 1 μg and 10 mg per kilogram body mass per minute.

### Medical Indications

In one aspect of the present invention there is provided an antibody or antibody fragment according to the invention as herein described for use in the treatment of a medical condition associated with a SARS Coronavirus, wherein the medical condition associated with a SARS Coronavirus is preferably COVID-19 or a SARS Coronavirus-associated respiratory disease.

As used herein, the “patient” or “subject” may be a vertebrate. In the context of the present invention, the term “subject” includes both humans and animals, particularly mammals, and other organisms.

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

In particular, the treatment described herein relates to either reducing or inhibiting Coronavirus infection or symptoms thereof via binding the viral Spike protein with the antibodies or fragments thereof of the present invention. The prophylactic therapy as described herein is intended to encompass prevention or reduction of risk of Coronavirus infection, due to a reduced likelihood of Coronavirus infection of cells via interaction with the ACE2 protein after treatment with the antibodies or fragments thereof described herein.

As used herein, a “patient with symptoms of an infectious disease” is a subject who presents with one or more of, without limitation, fever, diarrhea, fatigue, muscle aches, coughing, if have been bitten by an animal, having trouble breathing, severe headache with fever, rash or swelling, unexplained or prolonged fever or vision problems. Other symptoms may be fever and chills, very low body temperature, decreased output of urine (oliguria), rapid pulse, rapid breathing, nausea and vomiting. In preferred embodiments the symptoms of an infectious disease are fever, diarrhea, fatigue, muscle aches, rapid pulse, rapid breathing, nausea and vomiting and/or coughing. As used herein “infectious disease” comprises all diseases or disorders that are associated with bacterial and/or viral and/or fungal infections.

As used herein, a patient with “symptoms of a viral infection of the respiratory tract” is a subject who presents with one or more of, without limitation, cold-like symptoms or flu-like illnesses, such as fever, cough, runny nose, sneezing, sore throat, having trouble breathing, headache, muscle aches, fatigue, rapid pulse, rapid breathing, nausea and vomiting, lack of taste and/or smell and/or malaise (feeling unwell).

In some embodiments, symptoms of infection with a SARS-virus are fever, sore throat, cough, myalgia or fatigue, and in some embodiments, additionally, sputum production, headache, hemoptysis and/or diarrhea. In some embodiments, symptoms of an infection with a SARS-coronavirus, for example SARS-CoV-2, are fever, sore throat, cough, lack of taste and/or smell, shortness of breath and/or fatigue.

As used herein, the term “a patient that is at risk of developing a severe acute respiratory syndrome (SARS)” relates to a subject, preferably distinct from any given person in the general population, who has an increased (e.g. above-average) risk of developing SARS. In some embodiments, the patient has symptoms of SARS or symptoms of a SARS Coronavirus infection. In some embodiments, the patient has no symptoms of SARS or symptoms of a SARS Coronavirus infection. In some embodiments, the subject has been in contact with people with SARS Coronavirus infections or symptoms. In some embodiments, the person at risk of developing SARS has been tested for the presence of a SARS Coronavirus infection. In some embodiments, the person at risk of developing SARS has tested positive for the presence of a SARS Coronavirus infection, preferably a coronavirus infection.

In embodiments, the patient at risk of developing SARS is an asymptomatic patient that shows no specific symptoms of SARS (yet). An asymptomatic patient may be at risk of developing SARS because the patient has been in contact with a person infected with a SARS Coronavirus. For example, the asymptomatic patient may have been identified as being at risk of developing SARS by a software application (app) that is installed on his smart phone or corresponding (portable) device and that indicates physical proximity or short physical distance to an infected patient that uses a corresponding app on its respective mobile device/smart phone. Other methods of determining contact/physical proximity to an infected person are known to the skilled person and equally apply to the method of the invention.

In some embodiments, the patient that has or is at risk of developing a severe acute respiratory syndrome (SARS) has a coronavirus infection.

Coronaviruses are a group of related viruses that cause diseases in mammals and birds. The scientific name for coronavirus is Orthocoronavirinae or Coronavirinae. Coronavirus belongs to the family of Coronaviridae. The family is divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts.

In humans, coronaviruses cause respiratory tract infections that can be mild, such as some cases of the common cold, and others that can be lethal, such as SARS, MERS, and COVID-19. Coronaviruses are enveloped viruses with a positive-sense single-stranded RNA genome and a nucleocapsid of helical symmetry. The genome size of coronaviruses ranges from approximately 27 to 34 kilobases, the largest among known RNA viruses.

Various species of human coronaviruses are known, such as, without limitation, Human coronavirus OC43 (HCoV-OC43), of the genus β-CoV, Human coronavirus HKU1 (HCoV-HKU1), of the genus β-CoV, Human coronavirus 229E (HCoV-229E), α-CoV, Human coronavirus NL63 (HCoV-NL63), α-CoV, Middle East respiratory syndrome-related coronavirus (MERS-CoV), Severe acute respiratory syndrome coronavirus (SARS-CoV), Severe acute respiratory syndrome coronavirus 2 (SARS-CoV-2).

Coronaviruses vary significantly in risk factor. Some can kill more than 30% of those infected (such as MERS-CoV), and some are relatively harmless, such as the common cold. Coronaviruses cause colds with major symptoms, such as fever, and a sore throat, e.g. from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). Coronaviruses can also cause SARS.

Advances in nucleic acid sequencing technology (commonly termed Next-Generation Sequencing, NGS) are providing large sets of sequence data obtained from a variety of biological samples and allowing the characterization of both known and novel virus strains. Established methods are therefore available for determining a Coronavirus infection.

Viruses encode a collection of proteins required to ensure self-replication and persistence of the encoding virus. Enzymes for genome mRNA production and genome replication, proteases for protein maturation, proteins for genome encapsidation, and proteins for undermining the host antiviral responses can be identified conserved protein motifs or domains. Likely because of selective pressures, viral genomes are streamlined and the functional protein content encoded by viruses is much higher than for a cellular organisms. Thus, describing a viral genome by the collection of encoded protein domains is a potentially useful classification method. Viral evolution can therefore be followed and novel strains of coronavirus can be determined based on sequence compairon to known coronavirus strains.

In some embodiments, the patient suffers from an infection, preferably with a SARS Coronavirus (SARS-CoV). As used herein, SARS Coronavirus refers to a Coronavirus that leads to severe acute respiratory syndrome (SARS). This syndrome is a viral respiratory disease of zoonotic origin that first surfaced in the early 2000s caused by the first-identified strain of the SARS coronavirus (SARS-CoV or SARS-CoV-1).

SARS is induced via droplet transmission and replication of the virus could be shown in the upper and lower respiratory tract or gastrointestinal mucosa. In parallel the virus is also capable of directly invading cells of different organs such as liver, kidney, heart and brain. Another distinct mechanism appears to be the direct invasion of the virus in T-cells. A significant number of SARS patients, who suffer COVID-19, have a clinically low concentration of lymphocytes in the blood, also known as Lymphocytopenia. Clinically, patients show respiratory symptoms such as dry cough and shortness of breath, fever or diarrhea. But symptoms associated with acute liver injury, heart injury or kidney injury can also occur. In a less severe state of SARS, patients can show mild or even no symptoms.

Clinical and scientific investigations show that SARS-CoVs bind to the epithelial cells via the Angiotensin converting enzyme 2 receptor (ACE2). ACE2 is a cell membrane linked carboxypeptidase which is expressed in vascular endothelia, kidney, bladder, heart, nasal mucosa, bronchus and lung. As one consequence binding of the virus leads to an epithelial and endothelial cell damage along with vascular leakage, which triggers a secretion of pro-inflammatory cytokines and chemokines. The virus also mediates ACE2 downregulation and shedding which further promotes a dysfunctional renin-angiotensin system (RAS). Once the RAS is disturbed it can lead an inflammatory response and to vascular permeability. Focusing on the respiratory system, ACE2 shedding can lead to pulmonary vascular permeability and subsequently to pulmonary edema.

Older patients (above 60 years), patients with chronic diseases (e.g. cardiovascular diseases, diabetes, cancer, COPD) or immune compromised patients are considered to be at a higher risk to face a severe development of SARS. Smoking and obesity are also considered as risk factors.

Embodiments of a SARS Coronavirus include, without limitation, any coronavirus that induces a SARS or SARS-similar pathology. Particular embodiments include, without limitation, the SARS Coronavirus (SARS-CoV-1) first discovered in 2003 (as described above), the Middle East respiratory syndrome (MERS-CoV) first discovered in 2012, and the SARS-CoV-2, which causes COVID-19, a disease which brought about the 2019-2020 coronavirus pandemic.

The strain SARS-CoV-2 causes COVID-19, a disease which brought about the ongoing 2019-2020 coronavirus pandemic. The disease was first identified in December 2019 in Wuhan, the capital of China's Hubei province, and spread globally. Common symptoms include fever, cough, and shortness of breath. Other symptoms may include muscle pain, diarrhea, sore throat, loss of taste and/or smell, and abdominal pain. While the majority of cases result in mild symptoms, some progress to viral pneumonia and multi-organ failure.

Coronaviruses can be determined by molecular techniques, for example sequence-based analysis, for example by PCR-based amplification of viral genetic material. Genome-wide phylogenetic analysis indicates that SARS-CoV-2 shares 79.5% and 50% sequence identity to SARS-CoV and MERS-CoV, respectively. However, there is 94.6% sequence identity between the seven conserved replicase domains in ORF1ab of SARS-CoV-2 and SARS-CoV, and less than 90% sequence identity between those of SARS-CoV-2 and other -CoVs, implying that SARS-CoV-2 belongs to the lineage of Beta-CoVs.

Similar to other CoVs, the SARS-CoV-2 virion with a genome size of 29.9 kb possesses a nucleocapsid composed of genomic RNA and phosphorylated nucleocapsid (N) protein. The nucleocapsid is buried inside phospholipid bilayers and covered by two di erent types of spike proteins: the spike glycoprotein trimmer (S) that exists in all CoVs, and the hemagglutinin-esterase (HE) only shared among some CoVs. The membrane (M) protein and the envelope (E) protein are located among the S proteins in the viral envelope. The SARS-CoV-2 genome has 5′ and 3′ terminal sequences (265 nt at the 5′ terminal and 229 nt at the 3′ terminal region), which is typical of -CoVs, with a gene order 5′-replicase open reading frame (ORF) 1ab-S-envelope(E)-membrane(M)-N-30. The predicted S, ORF3a, E, M, and N genes of SARS-CoV-2 are 3822, 828, 228, 669, and 1260 nt in length, respectively. Similar to SARS-CoV, SARS-CoV-2 carries a predicted ORF8 gene (366 nt in length) located between the M and N ORF genes.

According to Jin et al (Viruses 2020, 12, 372), in the initial 41 patients, fever (98%), cough (76%), and myalgia or fatigue (44%) were the most common symptoms. Less common symptoms were sputum production (28%), headache (8%), hemoptysis (5%), and diarrhea (3%). More than half of patients developed dyspnea. The average incubation period and basic reproduction number (R0) were estimated to be 5.2 d (95% Cl: 4.1-7.0) and 2.2 (95% Cl, 1.4-3.9), respectively.

In some embodiments, subjects have been tested and determined to have a SARS-Cov. Various methods may be employed to detect SAR-CoV, e.g. SARS-CoV-2, such as a nucleic acid test, serologic diagnosis, CRISPR/Cas13-based SHERLOCK technology, or imaging technology, such as chest radiographs or CT-scans.

Multiple SARS-CoV-2 variants are circulating globally. Additional variants and mutants of SARS-CoV-2 are being discovered regularly. Additional variants comprise, without limitation:

Lineage B.1.1.7/Variant of Concern 20DEC-01: First detected in October 2020 during the COVID-19 pandemic in the United Kingdom Lineage B.1.1.7, was previously known as the first Variant Under Investigation in December 2020 (VUI-202012/01) and later notated as VOC-202012/01. It is also known as lineage B.1.1.7 or 201/501Y.V1 (formerly 20B/501Y.V1). As of May 2021, Lineage B.1.1.7 has been detected in some 120 countries.

Variant of Concern 21FEB-02: Previously written as VOC-202102/02, described by Public Health England (PHE) as “B.1.1.7 with E484K” is of the same lineage in the Pango nomenclature system, but has an additional E484K mutation.

Lineage B.1.1.207: First sequenced in August 2020 in Nigeria, this variant has a P681H mutation, shared in common with UK's Lineage B.1.1.7. It shares no other mutations with Lineage B.1.1.7 and as of late December 2020 this variant accounts for around 1% of viral genomes sequenced in Nigeria. As of May 2021, Lineage B.1.1.207 has been detected in 10 countries.

Lineage B.1.1.317: B.1.1.317 was identified in Queensland, Australia.

Lineage B.1.1.318: Lineage B.1.1.318 was designated by PHE as a VUI (VUI-21FEB-04, previously VUI-202102/04) on 24 Feb. 2021.

Lineage B.1.351: On 18 Dec. 2020, the 501.V2 variant, also known as 501.V2, 20H/501Y.V2 (formerly 20C/501Y.V2), VOC-20DEC-02 (formerly VOC-202012/02), or lineage B.1.351, was first detected in South Africa and reported by the country's health department. The South African health department also indicated that the variant may be driving the second wave of the COVID-19 epidemic in the country due to the variant spreading at a more rapid pace than other earlier variants of the virus. The variant contains several mutations that allow it to attach more easily to human cells because of the following three mutations in the receptor-binding domain (RBD) in the spike glycoprotein of the virus: N501Y, K417N, and E484K.

Lineage B.1.429/CAL.20C: Lineage B.1.429, also known as CAL.20C, is defined by five distinct mutations (I4205V and D1183Y in the ORF1ab-gene, and S13I, W152C, L452R in the spike protein's S-gene). B.1.429 was first observed in July 2020 by researchers at the Cedars-Sinai Medical Center, California, in one of 1,230 virus samples collected in Los Angeles County since the start of the COVID-19 epidemic.

Lineage B.1.525: B.1.525, also called VUI-21FEB-03[15] (previously VUI-202102/03) by Public Health England (PHE) and formerly known as UK1188, does not carry the same N501Y mutation found in B.1.1.7, 501. V2 and P.1, but carries the same E484K-mutation as found in the P.1, P.2, and 501.V2 variants, and also carries the same ΔH69/ΔV70 deletion (a deletion of the amino acids histidine and valine in positions 69 and 70) as found in B.1.1.7, N439K variant (B.1.141 and B.1.258) and Y453F variant (Cluster 5). B.1.525 differs from other variants by having both the E484K-mutation and a new F888L mutation (a substitution of phenylalanine (F) with leucine (L) in the S2 domain of the spike protein).

Lineage B.1.526: In November 2020, a mutant variant was discovered in New York City, which was named B.1.526.

Lineage B.1.617: In October 2020, a new variant was discovered in India, which was named B.1.617. Among some 15 defining mutations, it has spike mutations D111D (synonymous substitution), G142D, P681R, E484Q and L452R, the latter two of which may cause it to easily avoid antibodies. Public Health England (PHE) designated B.1.617 as a ‘Variant under investigation’, VUI-21APR-01. On 29 Apr. 2021, PHE added two further variants, VUI-21APR-02 and VUI-21APR-03, effectively B.1.617.2 and B.1.617.3. B.1.617.2 (which notably lacks mutation at E484Q) is a “variant of concern”.

Lineage B.1.618: This variant was first isolated in October 2020, and has the E484K mutation as in South African variant B.1.351.

Lineage B.1.620: In March 2021, this variant was discovered in Lithuania.

Lineage P.1: Lineage P.1, termed Variant of Concern 21JAN-02 (formerly VOC-202101/02) by Public Health England and 20J/501Y.V3 by Nextstrain, was detected in Tokyo on 6 Jan. 2021 by the National Institute of Infectious Diseases (NIID). This variant of SARS-CoV-2 has been named in the P.1 lineage, and has 17 unique amino acid changes, 10 of which in its spike protein, including N501Y, E484K and K417T.

Lineage P.3: On 18 Feb. 2021, the Department of Health of the Philippines confirmed the detection of two mutations of COVID-19 in Central Visayas after samples from patients were sent to undergo genome sequencing. The mutations were later named as E484K and N501Y.

In embodiments of the invention the antibodies or fragments described herein can bind and/or neutralize multiple SARS-CoV-2 variants, representing an unexpected and beneficial property of the invention.

### Spike and ACE2 Proteins

The Coronavirus Spike protein, also known as S protein, is a glycoprotein trimer, wherein each monomer of the trimeric S protein is about 180 kDa, and contains two subunits, S1 and S2, mediating attachment and membrane fusion, respectively. As described in Xiuyuan Ou et al (Nature Communications, 11, 1620, 2020), Coronaviruses (CoV) use the Spike glycoprotein to bind ACE2 and mediate membrane fusion and virus entry. In the S protein structure, N- and C-terminal portions of S1 fold as two independent domains, N-terminal domain (NTD) and C-terminal domain (C-domain). Depending on the virus, either the NTD or C-domain can serve as the receptor-binding domain (RBD). While RBD of mouse hepatitis virus (MHV) is located at the NTD14, most of other CoVs, including SARS-CoV and MERS-CoV use C-domain to bind their receptors.

Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level using cryogenic electron microscopy, is the protein responsible for allowing the virus to attach to and fuse with the membrane of a host cell.

The receptors for SARS-CoV and MERS-CoV are human angiotensin-converting enzyme 2 (hACE2) and human dipeptidyl peptidase 4 (hDPP4), respectively. CoV S proteins are typical class I viral fusion proteins, and protease cleavage is required for activation of the fusion potential of S protein. A two-step sequential protease cleavage model has been proposed for activation of S proteins of SARS-CoV and MERS-CoV, priming cleavage between S1 and S2 and activating cleavage on S2′ site. Depending on virus strains and cell types, CoV S proteins may be cleaved by one or several host proteases, including furin, trypsin, cathepsins, transmembrane protease serine protease-2 (TMPRSS-2), TMPRSS-4, or human airway trypsin-like protease (HAT). Availability of these proteases on target cells largely determines whether CoVs enter cells through plasma membrane or endocytosis.

In some embodiments, the antibody target is the receptor binding domain (RBD) of the S1 subunit (S1) of a recombinant Spike glycoprotein of SARS-CoV-2 (SARS-CoV-2-Spike-S1-RBD glycoprotein), as disclosed by the exemplary sequence SEQ ID NO 3. In some embodiments, the SARS-CoV-2-Spike-S1-RBD glycoprotein is expressed from HEK cells, thereby maintaining a human-similar glycosylation pattern.

Angiotensin-converting enzyme 2 (ACE2) is a cell membrane linked carboxypeptidase presented on the outer surface (cell membranes) of cells in the vascular endothelia, kidney, bladder, heart, nasal mucosa, bronchus and lung. ACE2 has the function of lowering blood pressure by catalyzing the hydrolysis of angiotensin II into angiotensin. ACE2 counters the activity of the related angiotensin-converting enzyme (ACE) making it a drug target for treating cardiovascular diseases. ACE2 is a which is expressed in. ACE2 also serves as the entry point into cells for some coronaviruses including Severe acute respiratory syndrome coronavirus 2.

ACE2 is a zinc containing metalloenzyme that contains an N-terminal peptidase M2 domain and a C-terminal collectrin renal amino acid transporter domain. ACE2 is a single-pass type I membrane protein, with its enzymatically active domain exposed on the surface of cells. The extracellular domain of ACE2 is cleaved from the transmembrane domain by another enzyme known as sheddase, and the resulting soluble protein is released into the blood stream and ultimately excreted into urine.

In one embodiment of the invention, a soluble form of ACE2 is presented in SEQ ID NO 7, which comprises the signal peptide of ACE2 and comprises amino acids 1-615 of the ACE2 protein.

### Protein Glycosylation

Protein glycosylation is post-translational modification (PTM) which is important for pharmacokinetics and immunogenicity of recombinant glycoproteins. As a result of variations in monosaccharide composition, glycosidic linkages and glycan branching, glycosylation introduces considerable complexity and heterogeneity to protein function and structure. The host cell line used to produce the glycoprotein has a strong influence on the glycosylation because different host systems may express varying repertoire of glycosylation enzymes and transporters that contributes to specificity and heterogeneity in glycosylation profiles.

Glycosylation typically occurs within the secretory pathways of cells, that is, endoplasmic reticulum (ER) and Golgi apparatus, where monosaccharide units such as galactose (Gal), mannose (Man), fucose (Fuc), N-acetylglucosamine (GlcNAc), N-acetylgalactosamine (GalNAc) and sialic acids are covalently attached to specific amino acids of newly synthesized proteins and lipid structures. Glycans attached to the amide nitrogen atom of asparagine (Asn) residues are termed N-linked glycans, while glycans attached to the oxygen atom of serine (Ser) or threonine (Thr) residues are O-linked glycans. Glycans can be attached in linear or branching chains and may be linked by α- or β-glycosidic linkages at various linkage positions. The possible variations in monosaccharide composition, glycosidic linkages and glycan branching gives rise to an extremely diverse glycan repertoire. Concomitantly, proteins with varying glycostructures may differ in structure and function, as glycans can significantly influence protein solubility, bioactivity, stability and immunogenicity.

According to the present invention, in some embodiments, the antibody target, namely the receptor binding domain (RBD) of the S1 subunit (S1) of a recombinant Spike glycoprotein of SARS-CoV-2 (SARS-CoV-2-Spike-S1-RBD glycoprotein) is expressed in human embryonic kidney (HEK293) cells, thereby producing the protein target of the inventive antibodies, the HEK-SARS-CoV-2-Spike-S1-RBD glycoprotein.

Due to expression in HEK cells, the Spike glycoprotein is likely to exhibit a glycosylation pattern similar to the Spike protein present in CoV virions after human infection, thereby enabling an effective binding and neutralization of the CoV using the antibodies or fragments thereof according to the present invention.

A skilled person can determine and/or predict the glycosylation of a protein form any given cell type. For example, glycosylation patterns can be predicted using software, for example the NetNGlyc 1.0 or 4.0 Server or N-GlyDE, a two-stage N-linked glycosylation site prediction incorporating gapped dipeptides and pattern-based encoding (Scientific Reports volume 9, Article number: 15975, 2019).

Glycopeptide bonds can be categorized into specific groups based on the nature of the sugar-peptide bond and the oligosaccharide attached, including N-, O- and C-linked glycosylation, glypiation and phosphoglycosylation. Recent advances have led to the development of new analytical methods that employ mass spectrometry extensively making it possible to obtain the glycosylation site and the site microheterogeneity (Curr Opin Chem Biol. 2009 October; 13(4): 421-426). Furthermore, glycan staining or labeling, glycoprotein purification or enrichment or glycoproteome and glycome analysis by mass spectrometry can be employed. For example, this approach can be performed before or after enzymatic cleavage of glycans via endoglycanase H (endo H) or peptide-N4-(N-acetyl-beta-glucosaminyl) asparagine amidase (PNGase), depending on the type of experiment. Quantitative comparative glycoproteome analysis can be performed by differential labeling with stable isotope labeling by amino acids in cell culture (SILAC) reagents. Additionally, absolute quantitation by selected reaction monitoring (SRM) can be performed on targeted glycoproteins using isotopically labeled, “heavy” reference peptides.

### Embodiments Relating to Diagnostic Products

In another advantageous embodiment an immunoassay is used in the detection of Coronavirus using the antibodies or fragments thereof of the present invention, to which end binding of the antibodies or fragments thereof of the present invention to a solid phase is envisaged.

Following addition of sample solution, virus in the patient's sample binds to the solid phase bound antibodies or fragments thereof of the present invention. The virus which is obtained e.g. from the serum of a patient and bound to the solid phase is subsequently detected using a label, or labelled reagent and optionally quantified, preferably using a further antibody directed against the Coronavirus, as disclosed herein. Thus, according to the invention, detection of the virus in this method is achieved using labelled reagents according to the well-known ELISA (Enzyme-Linked Immunosorbent Assay) technology. Labels according to the invention therefore comprise enzymes catalysing a chemical reaction which can be determined by optical means, especially by means of chromogenic substrates, chemiluminescent methods or fluorescent dyes. In another preferred embodiment the autoantibodies are detected by labelling with weakly radioactive substances in radioimmunoassays (RIA) wherein the resulting radioactivity is measured.

The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); lateral flow assays (LFA) and chemiluminescence assays (CL).

In another preferred embodiment of the invention, soluble or solid phase-bound antibodies or fragments thereof of the present invention are used to bind the virus. In a second reaction step, further antibodies, such as those of the present invention, directed against the virus are employed, said further antibodies being detectably labelled. The advantage of this embodiment lies in the use of ELISA technology usually available in laboratory facilities so that detection according to the invention can be established in a cost-effective manner. The further antibodies may be detectably coupled to fluorescein isothiocyanate (FITC). Much like the above-mentioned ELISA, the FITC technology represents a system that is available in many places and therefore allows smooth and low-cost establishment of the inventive detection in laboratory routine.

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm.

In another embodiment of the invention the antibodies or fragments thereof of the present invention in accordance with one or more of the sequences disclosed herein is bound to a solid phase. Binding of antibodies or fragments thereof of the present invention in accordance with one or more of the sequences disclosed herein to the solid phase can be effected via a spacer. All those chemical compounds having suitable structural and functional preconditions for spacer function can be used as spacers as long as they do not modify the binding behavior in such a way that binding of the virus to antibodies or fragments thereof of the present invention is adversely affected. In another preferred embodiment of the invention the molecule comprises a linker or spacer selected from the group of α-aminocarboxylic acids as well as homo-and heterooligomers thereof, α,ω-aminocarboxylic acids and branched homo- or heterooligomers thereof, other amino acids, as well as linear and branched homo- or heterooligomers; amino-oligoalkoxyalkylamines; maleinimidocarboxylic acid derivatives; oligomers of alkylamines; 4-alkylphenyl derivatives; 4-oligoalkoxyphenyl or 4-oligoalkoxyphenoxy derivatives; 4-oligoalkylmercaptophenyl or 4-oligoalkylmercaptophenoxy derivatives; 4-oligoalkylaminophenyl or 4-oligoalkylaminophenoxy derivatives; (oligoalkylbenzyl)phenyl or 4-(oligoalkylbenzyl)phenoxy derivatives, as well as 4-(oligoalkoxybenzyl)phenyl or 4-(oligoalkoxybenzyl)phenoxy derivatives; trityl derivatives; benzyloxyaryl or benzyloxyalkyl derivatives; xanthen-3-yloxyalkyl derivatives; (4-alkylphenyl)- or ω-(4-alkylphenoxy)alkanoic acid derivatives; oligoalkylphenoxyalkyl or oligo-alkoxyphenoxyalkyl derivatives; carbamate derivatives; amines; trialkylsilyl or dialkylalkoxysilyl derivatives; alkyl or aryl derivatives or combinations thereof.

In another embodiment of the invention, the antibodies or fragments thereof of the present invention are immobilized. More specifically, the solid phase-bound antibodies or fragments thereof of the present invention is bound to organic, inorganic, synthetic and/or mixed polymers, preferably agarose, cellulose, silica gel, polyamides and/or polyvinyl alcohols. In the meaning of the invention, immobilization is understood to involve various methods and techniques to fix the antibodies or fragments thereof of the present invention on specific carriers, e.g. according to WO 99/56126 or WO 02/26292. For example, immobilization can serve to stabilize the peptides so that their activity would not be reduced or adversely modified by biological, chemical or physical exposure, especially during storage or in single-batch use. Immobilization of the antibodies or fragments thereof of the present invention allows repeated use under technical or clinical routine conditions; furthermore, a sample-preferably blood components-can be reacted with at least one of the antibodies or fragments thereof of the present invention in a continuous fashion. In the meaning of the invention, three basic methods can be used for immobilization:
(i) Crosslinking: in crosslinking, the antibodies are fixed to one another without adversely affecting their activity. Advantageously, they are no longer soluble as a result of such crosslinking.
(ii) Binding to a carrier: binding to a carrier proceeds via adsorption, ionic binding or covalent binding, for example. Such binding may also take place inside microbial cells or liposomes or other membranous, closed or open structures. Advantageously, the antibodies are not adversely affected by such fixing. For example, multiple or continuous use of carrier-bound antibodies is possible with advantage in clinical diagnosis or therapy.
(iii) Inclusion: inclusion in the meaning of the invention especially proceeds in a semipermeable membrane in the form of gels, fibrils or fibers. Advantageously, encapsulated antibodies are separated from the surrounding sample solution by a semipermeable membrane in such a way that interaction with the virus still is possible. Various methods are available for immobilization, such as adsorption on an inert or electrically charged inorganic or organic carrier.

Another method is covalent binding to carrier materials. In addition, the carriers may have reactive groups forming homopolar bonds with amino acid side chains. Suitable groups in antibodies are carboxy, hydroxy and sulfide groups and especially the terminal amino groups of lysines. Aromatic groups offer the possibility of diazo coupling. Advantageously, a large number of antibodies can undergo direct covalent binding with polyacrylamide resins.

The invention also relates to a diagnostic kit for the determination of a Coronavirus infection, comprising antibodies in accordance with one or more of the sequences as disclosed herein. The diagnostic kit optionally includes instructions concerning combining the contents of the kit and/or providing a formulation for the detection of viral infection. For example, the instruction can be in the form of an instruction leaflet or other medium providing the user with information as to the type of method wherein the substances mentioned are to be used. Obviously, the information need not necessarily be in the form of an instruction leaflet, and the information may also be imparted via the Internet, for example. To a patient, one advantageous effect of such a kit is, for instance, that he or she, without directly addressing a physician, can determine the actual state of a disease.

### SEQUENCES

### Preferred Sequences of the Invention

| SEQ | | |
|---|---|---|
| ID | Info | Amino Acid Sequence (5-3) |
| 1 | SARS-CoV-2 | MSTMRLLTLALLFSCSVARAACRVQPTESIVRFPNITNLCPFGEVFNATRFASVYA |
| | Spike RBD-Fc | WNRKRISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKSNLKPF |
| | | ERDISTEIYQAGSTPCNGVEGFNCYFPLQSYGFQPTNGVGYQPYRVVVLSFELLH |
| | | APATVCGPKKSTNLVKNKCVNFNFNGLTGTGVLTESNKKFLPFQQFGRDIADTTD |
| | | AVRDPQTLEILDITPCSGSSTMVRSSKPTCPPPELLGGPSVFIFPPKPKDTLMISRT |
| | | PEVTCVVVDVSQDDPEVQFTWYINNEQVRTARPPLREQQFNSTIRVVSTLPIAHQD |
| | | WLRGKEFKCKVHNKALPAPIEKTISKARGQPLEPKVYTMGPPREELSSRSVSLTC |
| | | MINGFYPSDISVEWEKNGKAEDNYKTTPAVLDSDGSYFLYSKLSVPTSEWQRGDV |
| | | FTCSVMHEALHNHYTQKSISRSPGK |
| 2 | Signal peptide | MSTMRLLTLALLFSCSVARAAC |
| | of GluN1 | |
| 3 | RBD-SD1 | RVQPTESIVRFPNITNLCPFGEVFNATRFASVYAWNRKRISNCVADYSVLYNSASF |
| | region of | STFKCYGVSPTKLNDLCFTNVYADSFVIRGDEVRQIAPGQTGKIADYNYKLPDDFT |
| | SARS-CoV-2 | GCVIAWNSNNLDSKVGGNYNYLYRLFRKSNLKPFERDISTEIYQAGSTPCNGVEG |
| | Spike | FNCYFPLQSYGFQPTNGVGYQPYRVVVLSFELLHAPATVCGPKKSTNLVKNKCVN |
| | | FNFNGLTGTGVLTESNKKFLPFQQFGRDIADTTDAVRDPQTLEILDITPCS |
| 4 | Linker | GSSTMVRS |
| | between | |
| | RBD-SD1 and | |
| | rabbit Fc | |
| 5 | rabbit Fc from | SKPTCPPPELLGGPSVFIFPPKPKDTLMISRTPEVTCVVVDVSQDDPEVQFTWYIN |
| | vector pFuse- | NEQVRTARPPLREQQFNSTIRVVSTLPIAHQDWLRGKEFKCKVHNKALPAPIEKTIS |
| | rIgG-Fc1 | KARGQPLEPKVYTMGPPREELSSRSVSLTCMINGFYPSDISVEWEKNGKAEDNYK |
| | | TTPAVLDSDGSYFLYSKLSVPTSEWQRGDVFTCSVMHEALHNHYTQKSISRSPGK |
| 6 | ACE2-HA | MSSSSWLLLSLVAVTAAQSTIEEQAKTFLDKFNHEAEDLFYQSSLASWNYNTNITE |
| | | ENVQNMNNAGDKWSAFLKEQSTLAQMYPLQEIQNLTVKLQLQALQQNGSSVLSE |
| | | DKSKRLNTILNTMSTIYSTGKVCNPDNPQECLLLEPGLNEIMANSLDYNERLWAWE |
| | | SWRSEVGKQLRPLYEEYVVLKNEMARANHYEDYGDYWRGDYEVNGVDGYDYSR |
| | | GQLIEDVEHTFEEIKPLYEHLHAYVRAKLMNAYPSYISPIGCLPAHLLGDMWGRFW |
| | | TNLYSLTVPFGQKPNIDVTDAMVDQAWDAQRIFKEAEKFFVSVGLPNMTQGFWEN |
| | | SMLTDPGNVQKAVCHPTAWDLGKGDFRILMCTKVTMDDFLTAHHEMGHIQYDMA |
| | | YAAQPFLLRNGANEGFHEAVGEIMSLSAATPKHLKSIGLLSPDFQEDNETEINFLLK |
| | | QALTIVGTLPFTYMLEKWRWMVFKGEIPKDQWMKKWWEMKREIVGVVEPVPHDE |
| | | TYCDPASLFHVSNDYSFIRYYTRTLYQFQFQEALCQAAKHEGPLHKCDISNSTEAG |
| | | QKLFNMLRLGKSEPWTLALENVVGAKNMNVRPLLNYFEPLFTWLKDQNKNSFVG |
| | | WSTDWSPYADHHHHHHHHGYPYDVPDYA |
| 7 | Amino acids | MSSSSWLLLSLVAVTAAQSTIEEQAKTFLDKFNHEAEDLFYQSSLASWNYNTNITE |
| | 1-615 of | ENVQNMNNAGDKWSAFLKEQSTLAQMYPLQEIQNLTVKLQLQALQQNGSSVLSE |
| | ACE2 | DKSKRLNTILNTMSTIYSTGKVCNPDNPQECLLLEPGLNEIMANSLDYNERLWAWE |
| | including | SWRSEVGKQLRPLYEEYVVLKNEMARANHYEDYGDYWRGDYEVNGVDGYDYSR |
| | signal peptide | GQLIEDVEHTFEEIKPLYEHLHAYVRAKLMNAYPSYISPIGCLPAHLLGDMWGRFW |
| | | TNLYSLTVPFGQKPNIDVTDAMVDQAWDAQRIFKEAEKFFVSVGLPNMTQGFWEN |
| | | SMLTDPGNVQKAVCHPTAWDLGKGDFRILMCTKVTMDDFLTAHHEMGHIQYDMA |
| | | YAAQPFLLRNGANEGFHEAVGEIMSLSAATPKHLKSIGLLSPDFQEDNETEINFLLK |
| | | QALTIVGTLPFTYMLEKWRWMVFKGEIPKDQWMKKWWEMKREIVGVVEPVPHDE |
| | | TYCDPASLFHVSNDYSFIRYYTRTLYQFQFQEALCQAAKHEGPLHKCDISNSTEAG |
| | | QKLFNMLRLGKSEPWTLALENVVGAKNMNVRPLLNYFEPLFTWLKDQNKNSFVG |
| | | WSTDWSPYAD |
| 8 | Polyhistidine | HHHHHHHH |
| | tag | |
| 9 | Hemagglutinin | YPYDVPDYA |
| | tag | |
| 10 | HL CV07-250 | GYNFTNFA |
| | H-CDR1 | |
| 11 | HL CV07-250 | ISGYNGDT |
| | H-CDR2 | |
| 12 | HL CV07-250 | AGSDNYGFPYNGMDV |
| | H-CDR3 | |
| 13 | HL CV07-250 | SSDLGAYHF |
| | L-CDR1 | |
| 14 | HL CV07-250 | GVR |
| | L-CDR2 | |
| 15 | HL CV07-250 | SSYAGNNDFV |
| | L-CDR3 | |
| 16 | HL CV07-250 | MGWSCIILFLVATATGVHSQVQLVQSEAEVKEPGASVEVSCKASGYNFTNFAISW |
| | VH | VRQAPGQGLEWMGWISGYNGDTNSAQKFLGRVTMTTDTSTTTAYMELRSLRSD |
| | | DTAVYYCAGSDNYGFPYNGMDVWGQGTTVTVSSASTKGPSVFPLAPSSKSTSGG |
| | | TAALGCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 17 | HL CV07-250 | MGWSCIILFLVATATGSWAQSALTQPPSASGSPGQSVTISCTGTSSDLGAYHFVT |
| | VL | WYQHYPGKAPKVMIYGVRKRPSGVPDRFSGSKSGNTASLTVSGLQDEDEADYYC |
| | | SSYAGNNDFVFGGGTKLTVLSQPKAAPSVTLFPPSSEELQANKATLVCLISDFYPG |
| | | AVTVAWKADSSPVKAGVETTTPSKQSNNKYAASSYLSLTPEQWKSHRSYSCQVT |
| | | HEGSTVEKTVAPTECS |
| 18 | HL CV07-270 | GFTFSDYY |
| | H-CDR1 | |
| 19 | HL CV07-270 | ISSSGSTI |
| | H-CDR2 | |
| 20 | HL CV07-270 | ARARGSSGWYRIGTRWGNWFDP |
| | H-CDR3 | |
| 21 | HL CV07-270 | SSDVGGYNY |
| | L-CDR1 | |
| 22 | HL CV07-270 | EVS |
| | L-CDR2 | |
| 23 | HL CV07-270 | SSYTSSSNVV |
| | L-CDR3 | |
| 24 | HL CV07-270 | MGWSCIILFLVATATGVHSQVQLVESGGGLVKPGGSLRLSCAASGFTFSDYYMTW |
| | VH | IRQAPGKGLEWVSYISSSGSTIYYADSVKGRFTISRDNAKNSLYLQMNSLRAEDTA |
| | | VYYCARARGSSGWYRIGTRWGNWFDPWGQGTLVTVSSASTKGPSVFPLAPSSK |
| | | STSGGTAALGCLVKDYFPEPVTVSWNSGALTSGVHTFPAVLQSSGLYSLSSVVTV |
| | | PSSSLGTQTYICNVNHKPSNTKVDKRVEPKSCDKTHTCPPCPAPELLGGPSVFLFP |
| | | PKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 25 | HL CV07-270 | MGWSCIILFLVATATGSWAQSALTQPASVSGSPGQSITISCTGTSSDVGGYNYVS |
| | VL | WYQQHPGKAPKLMIYEVSNRPSGVSNRFSGSKSGNTASLTISGLQAEDEADYYCS |
| | | SYTSSSNVVFGGGTMLTVLGQPKAAPSVTLFPPSSEELQANKATLVCLISDFYPGA |
| | | VTVAWKADSSPVKAGVETTTPSKQSNNKYAASSYLSLTPEQWKSHRSYSCQVTH |
| | | EGSTVEKTVAPTECS |
| 26 | HL CV07-283 | GYTFTGYY |
| | H-CDR1 | |
| 27 | HL CV07-283 | INPISGGT |
| | H-CDR2 | |
| 28 | HL CV07-283 | VRGPFYYDSSGPLGGMDV |
| | H-CDR3 | |
| 29 | HL CV07-283 | SGDVGGYNY |
| | L-CDR1 | |
| 30 | HL CV07-283 | EVS |
| | L-CDR2 | |
| 31 | HL CV07-283 | SSYTSSSTYV |
| | L-CDR3 | |
| 32 | HL CV07-283 | MGWSCIILFLVATATGVHSQVQLVQSGAEVKKPGASAKVSCKASGYTFTGYYIHW |
| | VH | VRQAPGQGLEWMGLINPISGGTNYAQKFQDRVTMTRDTSISTAYMELSRLRSDDT |
| | | AVYYCVRGPFYYDSSGPLGGMDVWGQGTTVTVSSASTKGPSVFPLAPSSKSTSG |
| | | GTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 33 | HL CV07-283 | MGWSCIILFLVATATGSWAQSALTQPASVSGSPGQSITISCTGTSGDVGGYNYVS |
| | VL | WYQQHPGKAPKLMIYEVSNRPSGVSNRFSGSKSGNTASLTISGLQAEDEADYYCS |
| | | SYTSSSTYVFGTGTKVTVLGQPKANPTVTLFPPSSEELQANKATLVCLISDFYPGA |
| | | VTVAWKADSSPVKAGVETTTPSKQSNNKYAASSYLSLTPEQWKSHRSYSCQVTH |
| | | EGSTVEKTVAPTECS |
| 34 | HK CV07-287 | GFTFTSSA |
| | H-CDR1 | |
| 35 | HK CV07-287 | IVVGSGNT |
| | H-CDR2 | |
| 36 | HK CV07-287 | AAPYCSSTNCYDAFDI |
| | H-CDR3 | |
| 37 | HK CV07-287 | QSVSSSY |
| | L-CDR1 | |
| 38 | HK CV07-287 | GAS |
| | L-CDR2 | |
| 39 | HK CV07-287 | QQYGSSPWT |
| | L-CDR3 | |
| 40 | HK CV07-287 | MGWSCIILFLVATATGVHSQVQLVQSGPEVKKPGTSVKVSCKASGFTFTSSAMQW |
| | VH | VRQARGQRLEWIGWIVVGSGNTNYAQKFQERVTITRDMSTSTAYMELSSLRSEDT |
| | | AVYYCAAPYCSSTNCYDAFDIWGQGTMVTVSSASTKGPSVFPLAPSSKSTSGGTA |
| | | ALGCLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 41 | HK CV07-287 | MGWSCIILFLVATATGVHSEIVLTQSPGTLSLSPGERATLSCRASQSVSSSYLAWY |
| | VL | QQKPGQAPRLLIYGASSRATGIPDRFSGSGSGTDFTLTISRLEPEDFAVYYCQQYG |
| | | SSPWTFGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGL |
| | | SSPVTKSFNRGEC |
| 42 | HK CV05-163 | GYTFTGYY |
| | H-CDR1 | |
| 43 | HK CV05-163 | INPNSGGT |
| | H-CDR2 | |
| 44 | HK CV05-163 | AREVMVRGALPPYGMDV |
| | H-CDR3 | |
| 45 | HK CV05-163 | QSVSSY |
| | L-CDR1 | |
| 46 | HK CV05-163 | DAS |
| | L-CDR2 | |
| 47 | HK CV05- | QQRSNWPPVT |
| | 1631 L-CDR3 | |
| 48 | HK CV05-163 | MGWSCIILFLVATATGVHSQVQLVQSGAEVKKPGASVKVSCKASGYTFTGYYMH |
| | VH | WVRQAPGQGLEWMGWINPNSGGTNYAQKFQGRVTMTRDTSISTAYMELSRLRS |
| | | DDTAVYYCAREVMVRGALPPYGMDVWGQGTTVTVSSASTKGPSVFPLAPSSKST |
| | | SGGTAALGCLVKDYFPEPVTVSWNSGALTSGVHTFPAVLQSSGLYSLSSVVTVPS |
| | | SSLGTQTYICNVNHKPSNTKVDKRVEPKSCDKTHTCPPCPAPELLGGPSVFLFPPK |
| | | PKDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 49 | HK CV05-163 | MGWSCIILFLVATATGVHSEIVLTQSPATLSLSPGERATLSCRASQSVSSYLAWYQ |
| | VL | QKPGQAPRLLIYDASNRATGIPARFSGSGSGTDFTLTISSLEPEDFAVYYCQQRSN |
| | | WPPVTFGGGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGL |
| | | SSPVTKSFNRGEC |
| 50 | HK CV07-209 | GFTFSDYY |
| | H-CDR1 | |
| 51 | HK CV07-209 | ISSSVSTI |
| | H-CDR2 | |
| 52 | HK CV07-209 | ARDGVIPPRFDY |
| | H-CDR3 | |
| 53 | HK CV07-209 | QDISNY |
| | L-CDR1 | |
| 54 | HK CV07-209 | DAS |
| | L-CDR2 | |
| 55 | HK CV07-209 | QQYDNLPLT |
| | L-CDR3 | |
| 56 | HK CV07-209 | MGWSCIILFLVATATGVHSQVQLVESGGGLVKPGGSLRLSCAASGFTFSDYYMSW |
| | VH | IRQAPGKGLEWVSYISSSVSTIYYADSVKGRFTISRDNAKNSLYLQMNSLRAEDTA |
| | | VYYCARDGVIPPRFDYWGQGTLVTVSSASTKGPSVFPLAPSSKSTSGGTAALGCL |
| | | VKDYFPEPVTVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGK |
| 57 | HK CV07-209 | MGWSCIILFLVATATGVHSDIQMTQSPSSLSASVGDRVTITCQASQDISNYLNWYQ |
| | VL | QKPGKAPKLLIYDASNLETGVPSRFSGSGSGTDFTFTISSLQPEDIATYYCQQYDN |
| | | LPLTFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 58 | HL CV07-222 | GYTFTDYY |
| | H-CDR1 | |
| 59 | HL CV07-222 | INPISGGT |
| | H-CDR2 | |
| 60 | HL CV07-222 | ARGPYYYDSSGSLGAFDI |
| | H-CDR3 | |
| 61 | HL CV07-222 | SSDVGSYN |
| | L-CDR1 | |
| 62 | HL CV07-222 | EGS |
| | L-CDR2 | |
| 63 | HL CV07-222 | CSYAGGSTSYV |
| | L-CDR3 | |
| 64 | HL CV07-222 | MGWSCIILFLVATATGVHSQVQLVQSGAEVKKPGASVKVSCKASGYTFTDYYVHW |
| | VH | VRQAPGQGLEWMGWINPISGGTNYAQKFQGRVTMTRDTSISTAYMELSRLRSDD |
| | | TAVYYCARGPYYYDSSGSLGAFDIWGQGTMVTVSSASTKGPSVFPLAPSSKSTSG |
| | | GTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 65 | HL CV07-222 | MGWSCIILFLVATATGSWAQSALTQPASVSGSPGQSITISCTGTSSDVGSYNLVSW |
| | VL | YQQHPGKAPKLMIYEGSKRPSGVSNRFSGSKSGNTASLTISGLQAEDEAHYYCCS |
| | | YAGGSTSYVFGTGTKVTVLGQPKANPTVTLFPPSSEELQANKATLVCLISDFYPGA |
| | | VTVAWKADSSPVKAGVETTTPSKQSNNKYAASSYLSLTPEQWKSHRSYSCQVTH |
| | | EGSTVEKTVAPTECS |
| 66 | HL CV07-262 | GYTFTGYY |
| | H-CDR1 | |
| 67 | HL CV07-262 | INPNSGGT |
| | H-CDR2 | |
| 68 | HL CV07-262 | ARVGWYDFGTPGDYYYYYGMDV |
| | H-CDR3 | |
| 69 | HL CV07-262 | SSDVGSYNL |
| | L-CDR1 | |
| 70 | HL CV07-262 | EGS |
| | L-CDR2 | |
| 71 | HL CV07-262 | CSYAGTSTFV |
| | L-CDR3 | |
| 72 | HL CV07-262 | MGWSCIILFLVATATGVHSQVQLVQSGAEVKKPGASVKVSCKASGYTFTGYYMH |
| | VH | WVRQAPGQGLEWMGWINPNSGGTNYAQTFQGRVTMTRDTSITTTYMELSRLRS |
| | | DDTAVYYCARVGWYDFGTPGDYYYYYGMDVWGQGTTVTVSSASTKGPSVFPLA |
| | | PSSKSTSGGTAALGCLVKDYFPEPVTVSWNSGALTSGVHTFPAVLQSSGLYSLSS |
| | | VVTVPSSSLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 73 | HL CV07-262 | MGWSCIILFLVATATGSWAQSALTQPASVSGSPGQSITISCTGTSSDVGSYNLVSW |
| | VL | YQQHPGKAPKLMIYEGSKRPSGVSNRFSGSKSGNTASLTISGLQAEDEADYFCCS |
| | | YAGTSTFVFGTGTKVTVLGQPKANPTVTLFPPSSEELQANKATLVCLISDFYPGAV |
| | | TVAWKADSSPVKAGVETTTPSKQSNNKYAASSYLSLTPEQWKSHRSYSCQVTHE |
| | | GSTVEKTVAPTECS |
| 74 | HK CV38-113 | GFTVSSNY |
| | H-CDR1 | |
| 75 | HK CV38-113 | IYSGGST |
| | H-CDR2 | |
| 76 | HK CV38-113 | ARGGRLADAAGDY |
| | H-CDR3 | |
| 77 | HK CV38-113 | QDISNY |
| | L-CDR1 | |
| 78 | HK CV38-113 | DAS |
| | L-CDR2 | |
| 79 | HK CV38-113 | QQYDNLPSWT |
| | L-CDR3 | |
| 80 | HK CV38-113 | MGWSCIILFLVATATGVHSEVQLVESGGGLVQPGGSLRLSCAASGFTVSSNYMSW |
| | VH | VRQAPGKGLEWVSVIYSGGSTYYADSVKGRFTISRHNSKNTLYLQMNSLRAEDTA |
| | | VYFCARGGRLADAAGDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 81 | HK CV38-113 | MGWSCIILFLVATATGVHSDIQMTQSPSSLSASVGDRVTITCQASQDISNYLNWYQ |
| | VL | QKPGKAPKLLIYDASNLETGVPSRFSGSGSGTDFTFTISSLQPEDIATYYCQQYDN |
| | | LPSWTFGQGTKLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVTHQGLSS |
| | | PVTKSFNRGEC |
| 82 | HL CV38-183 | GFTVSSNY |
| | H-CDR1 | |
| 83 | HL CV38-183 | IYSGGST |
| | H-CDR2 | |
| 84 | HL CV38-183 | ARGDGWDNYYYGMDV |
| | H-CDR3 | |
| 85 | HL CV38-183 | SSNIGAGYD |
| | L-CDR1 | |
| 86 | HL CV38-183 | ANS |
| | L-CDR2 | |
| 87 | HL CV38-183 | QSYDSSLSGSV |
| | L-CDR3 | |
| 88 | HL CV38-183 | MGWSCIILFLVATATGVHSEVQLVESGGGLIQPGGSLRLSCAASGFTVSSNYMSW |
| | VH | VRQAPGKGLEWVSVIYSGGSTYYADSVKGRFTISRDNSKNTLYLQMNSLRAEDTA |
| | | VYYCARGDGWDNYYYGMDVWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 89 | HL CV38-183 | MGWSCIILFLVATATGSWAQSVLTQPPSVSGAPGQRVTISCTGSSSNIGAGYDVH |
| | VL | WYQHLPGTAPKLLIYANSNRPSGVPDRFSGSKSGTSASLAITGLQAEDEADYYCQ |
| | | SYDSSLSGSVFGGGTKLTVLGQPKAAPSVTLFPPSSEELQANKATLVCLISDFYPG |
| | | AVTVAWKADSSPVKAGVETTTPSKQSNNKYAASSYLSLTPEQWKSHRSYSCQVT |
| | | HEGSTVEKTVAPTECS |
| 90 | HL CV07-255 | GYTFTGYY |
| | H-CDR1 | |
| 91 | HL CV07-255 | INPNSGGT |
| | H-CDR2 | |
| 92 | HL CV07-255 | ARDSRFSYVNGEFDY |
| | H-CDR3 | |
| 93 | HL CV07-255 | SSDVESYNL |
| | L-CDR1 | |
| 94 | HL CV07-255 | EVS |
| | L-CDR2 | |
| 95 | HL CV07-255 | CSYAGHSTWV |
| | L-CDR3 | |
| 96 | HL CV07-255 | MGWSCIILFLVATATGVHSQVQLVQSGAEVKKPEASVKVSCKASGYTFTGYYMHW |
| | VH | VRQAPGQGLEWMGWINPNSGGTNYAQKFQGRVTMTRDTSISTVYMELSRLRSD |
| | | DTAVYYCARDSRFSYVNGEFDYWGQGILVTVSSASTKGPSVFPLAPSSKSTSGGT |
| | | AALGCLVKDYFPEPVTVSWNSGALTSGVHTFPAVLQSSGLYSLSSVVTVPSSSLG |
| | | TQTYICNVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 97 | HL CV07-255 | MGWSCIILFLVATATGSWAQSALTQPASVSGSPGQSITISCTRTSSDVESYNLVSW |
| | VL | YQQHPGKAPKLMIYEVSKRPSGISNRFSGSKSGNTASLTISGLQAEDEADYYCCSY |
| | | AGHSTWVFGGGTELTVLGQPKAAPSVTLFPPSSEELQANKATLVCLISDFYPGAVT |
| | | VAWKADSSPVKAGVETTTPSKQSNNKYAASSYLSLTPEQWKSHRSYSCQVTHEG |
| | | STVEKTVAPTECS |
| 98 | HL CV07-315 | GFTFDDYA |
| | H-CDR1 | |
| 99 | HL CV07-315 | ISWNSGSI |
| | H-CDR2 | |
| 100 | HL CV07-315 | AKDFLWDLHPPRYYGMDV |
| | H-CDR3 | |
| 101 | HL CV07-315 | SSNIGNNY |
| | L-CDR1 | |
| 102 | HL CV07-315 | ENN |
| | L-CDR2 | |
| 103 | HL CV07-315 | GTWDSSLNGV |
| | L-CDR3 | |
| 104 | HL CV07-315 | MGWSCIILFLVATATGVHSEVQLVESGGGLVQPGRSLRLSCAASGFTFDDYAMHW |
| | VH | VRQAPGKGLEWVSGISWNSGSIGYADSVKGRFTISRDNAKNSLYLQMNSLRAEDT |
| | | ALYYCAKDFLWDLHPPRYYGMDVWGQGTTVTVSSASTKGPSVFPLAPSSKSTSG |
| | | GTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 105 | HL CV07-315 | MGWSCIILFLVATATGSWAQSVLTRPPSVSAAPGQKVTISCSGSSSNIGNNYVSW |
| | VL | YQQLPGTAPKLLIYENNKRPSGIPDRFSGSKSGTSATLGITGLQTGDEADYYCGTW |
| | | DSSLNGVFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSCQVTHEG |
| | | STVEKTVAPTECS |
| 106 | HK CV38-139 | GFTVSSNY |
| | H-CDR1 | |
| 107 | HK CV38-139 | FYSGGST |
| | H-CDR2 | |
| 108 | HK CV38-139 | ARGHYDLFDY |
| | H-CDR3 | |
| 109 | HK CV38-139 | QGISSY |
| | L-CDR1 | |
| 110 | HK CV38-139 | AAS |
| | L-CDR2 | |
| 111 | HK CV38-139 | QQLNSYPPGT |
| | L-CDR3 | |
| 112 | HK CV38-139 | MGWSCIILFLVATATGVHSEVQLVESGGGLVQPGGSLRLSCAASGFTVSSNYMSW |
| | VH | VRQAPGKGLEWVSVFYSGGSTFYADSVKGRFTISRDNSKNTLYLQMNSLRAEDTA |
| | | VYYCARGHYDLFDYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYICNVN |
| | | HKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVF |
| | | SCSVMHEALHNHYTQKSLSLSPGK |
| 113 | HK CV38-139 | MGWSCIILFLVATATGVHSDIQLTQSPSFLSASVGDRVTITCRASQGISSYLAWYQQ |
| | VL | KPGKAPKLLIYAASTLQSGVPSRFSGSGSGTEFTLTISSLQPEDFATYYCQQLNSY |
| | | PPGTFGPGTKVDIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 114 | HK CV38-142 | GYSFTSYW |
| | H-CDR1 | |
| 115 | HK CV38-142 | IYPGESDT |
| | H-CDR2 | |
| 116 | HK CV38-142 | ARIRGVYSSGWIGGDY |
| | H-CDR3 | |
| 117 | HK CV38-142 | QSISSY |
| | L-CDR1 | |
| 118 | HK CV38-142 | AAS |
| | L-CDR2 | |
| 119 | HK CV38-142 | QQSYSTPRQWT |
| | L-CDR3 | |
| 120 | HK CV38-142 | MGWSCIILFLVATATGVHSEVQLVQSGAEVKKPGESLKISCQGSGYSFTSYWIGW |
| | VH | VRQMPGKGLEWMGIIYPGESDTRYSSSFQGHVTISADKSISTAYLQWSSLKASDT |
| | | AMYYCARIRGVYSSGWIGGDYWGQGTLVTVSSASTKGPSVFPLAPSSKSTSGGT |
| | | AALGCLVKDYFPEPVTVSWNSGALTSGVHTFPAVLQSSGLYSLSSVVTVPSSSLG |
| | | TQTYICNVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 121 | HK CV38-142 | MGWSCIILFLVATATGVHSDIQMTQSPSSLSASVGDRVTITCRASQSISSYLNWYQ |
| | VL | QKPGKAPKLLIYAASSLQSGVPSRFSGSGSGTDFTLTISSLQPEDFATYYCQQSYS |
| | | TPRQWTFGQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGL |
| | | SSPVTKSFNRGEC |
| 122 | HK CVX1-126 | GLTVSSNY |
| | H-CDR1 | |
| 123 | HK CVX1-126 | IYSGGST |
| | H-CDR2 | |
| 124 | HK CVX1-126 | ARDLSEGGMDV |
| | H-CDR3 | |
| 125 | HK CVX1-126 | QGISSW |
| | L-CDR1 | |
| 126 | HK CVX1-126 | AAS |
| | L-CDR2 | |
| 127 | HK CVX1-126 | QQANGFPPL |
| | L-CDR3 | |
| 128 | HK CVX1-126 | MGWSCIILFLVATATGVHSEVQLVESGGGLVQPGGSLRLSCAASGLTVSSNYMSW |
| | VH | VRQAPGKGLEWVSVIYSGGSTYYADSVKGRFTISRHNSKNTLYLQMNSVRAEDTA |
| | | VYYCARDLSEGGMDVWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGK |
| 129 | HK CVX1-126 | IMGWSCIILFLVATATGVHSDIQMTQSPSSVSASVGDRVTITCRASQGISSWLAWYQ |
| | VL | QKPGKAPKLLIYAASTLQSGVPSRFSGSGSGTDFTLTISSLQPEDFATYYCQQANG |
| | | FPPLFGGGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 130 | HK CV38-221 | GITVSSNY |
| | H-CDR1 | |
| 131 | HK CV38-221 | IYSGGST |
| | H-CDR2 | |
| 132 | HK CV38-221 | ARGFGDYYFDY |
| | H-CDR3 | |
| 133 | HK CV38-221 | QSVSSSY |
| | L-CDR1 | |
| 134 | HK CV38-221 | GAS |
| | L-CDR2 | |
| 135 | HK CV38-221 | QQLYT |
| | L-CDR3 | |
| 136 | HK CV38-221 | MGWSCIILFLVATATGVHSEVQLVESGGGLVQPGGSLRLSCAASGITVSSNYMIWV |
| | VH | RQAPGKGLEWVSVIYSGGSTYYADSVKGRFTISRDNSKNTLYLQMNSLRAEDTAV |
| | | YYCARGFGDYYFDYWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQGNV |
| | | FSCSVMHEALHNHYTQKSLSLSPGK |
| 137 | HK CV38-221 | MGWSCIILFLVATATGVHSEIVLTQSPGTLSLSPGERATLSCRASQSVSSSYFAWY |
| | VL | QQKPGQAPRLLIYGASSRATGIPDRFSGSGSGTDFTLTISRLEPEDFAVYYCQQLY |
| | | TFGQGTKLEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSSPVTK |
| | | SFNRGEC |
| 138 | HK CVX2-106 | GGTFSSYA |
| | H-CDR1 | |
| 139 | HK CVX2-106 | IIPIFGTA |
| | H-CDR2 | |
| 140 | HK CVX2-106 | ATRKETTVTTSLVYGMDV |
| | H-CDR3 | |
| 141 | HK CVX2-106 | QSISSF |
| | L-CDR1 | |
| 142 | HK CVX2-106 | AAS |
| | L-CDR2 | |
| 143 | HK CVX2-106 | QQSYSTPYT |
| | L-CDR3 | |
| 144 | HK CVX2-106 | MGWSCIILFLVATATGVHSQVQLVQSGAEVKKPGSSVKVSCKASGGTFSSYAISW |
| | VH | VRQAPGQGLEWMGGIIPIFGTANYAQKFQGRVTITADESTSTAYMELSSLRSEDTA |
| | | VYYCATRKETTVTTSLVYGMDVWGQGTTVTVSSASTKGPSVFPLAPSSKSTSGGT |
| | | AALGCLVKDYFPEPVTVSWNSGALTSGVHTFPAVLQSSGLYSLSSVVTVPSSSLG |
| | | TQTYICNVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 145 | HK CVX2-106 | MGWSCIILFLVATATGVHSDIQMTQSPSSLSASVGDRVTITCRASQSISSFLNWYQ |
| | VL | QKPGKAPKLLIYAASSLQSGVPSRFSGSGSVTDFTLTISSLQPEDFATYYCQQSYS |
| | | TPYTFGQGTKLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSP |
| | | VTKSFNRGEC |
| 146 | HK CV07-202 | GFTVSSNY |
| | H-CDR1 | |
| 147 | HK CV07-202 | IYSGGST |
| | H-CDR2 | |
| 148 | HK CV07-202 | ARDLYYYGMDV |
| | H-CDR3 | |
| 149 | HK CV07-202 | QGISSY |
| | L-CDR1 | |
| 150 | HK CV07-202 | AAS |
| | L-CDR2 | |
| 151 | HK CV07-202 | QQLNNYSVT |
| | L-CDR3 | |
| 152 | HK CV07-202 | MGWSCIILFLVATATGVHSEVQLVESGGGLIQPGGSLRLSCAASGFTVSSNYMSW |
| | VH | VRQAPGKGLEWISVIYSGGSTFYADSVKGRFTISRDNSKNTLYLQMNSLRAEDTAV |
| | | YYCARDLYYYGMDVWGQGTTVTVSSASTKGPSVFPLAPSSKSTSGGTAALGCLV |
| | | KDYFPEPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQGNV |
| | | FSCSVMHEALHNHYTQKSLSLSPGK |
| 153 | HK CV07-202 | MGWSCIILFLVATATGVHSDIQLTQSPSFLSASVGDRVTITCRASQGISSYLAWYQQ |
| | VL | KPGKAPKLLIYAASTLQSGVPSRFSGSGSGTEFTLTISSLQPEDFATYYCQQLNNY |
| | | SVTFGGGTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQGLSSPV |
| | | TKSFNRGEC |
| 154 | HL CV07-267 | GYTFTGYY |
| | H-CDR1 | |
| 155 | HL CV07-267 | INPNSGGT |
| | H-CDR2 | |
| 156 | HL CV07-267 | ARVFGPGLDCSSTSCYTYGMDV |
| | H-CDR3 | |
| 157 | HL CV07-267 | SSDVGSYNL |
| | L-CDR1 | |
| 158 | HL CV07-267 | EVS |
| | L-CDR2 | |
| 159 | HL CV07-267 | CSYAGSSSWV |
| | L-CDR3 | |
| 160 | HL CV07-267 | MGWSCIILFLVATATGVHSQVQLVQSGAEVKKPGASVKVSCKASGYTFTGYYMH |
| | VH | WVRQAPGQGLEWMGWINPNSGGTNSAQKFQGRVTMTRDTSITTAYMELSRLRS |
| | | DDTAVYYCARVFGPGLDCSSTSCYTYGMDVWGQGTTVTVSSASTKGPSVFPLAP |
| | | SSKSTSGGTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTL |
| | | PPSREEMTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 161 | HL CV07-267 | MGWSCIILFLVATATGSWAQSALTQPASVSGSPGQSITISCTGTSSDVGSYNLVSW |
| | VL | YQQHPGKAPKLMIYEVSKRPSGVSNRFSGSKSGNTASLTISGLQAEDEADYYCCS |
| | | YAGSSSWVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYSCQVTHE |
| | | GSTVEKTVAPTECS |
| 162 | RBD-SD1 | RVQPTESIVRFPNITNLCPFGEVFNATRFASVYAWNRKRISNCVADYSVLYNSASF |
| | region of | STFKCYGVSPTKLNDLCFTNVYADSFVIRGDEVRQIAPGQTGKIADYNYKLPDDFT |
| | SARS-CoV-2 | GCVIAWNSNNLDSKVGGNYNYLYRLFRKSNLKPFERDISTEIYQAGSTPCNGVEG |
| | B.1.1.7 Spike | FNCYFPLQSYGFQPTYGVGYQPYRVVVLSFELLHAPATVCGPKKSTNLVKNKCVN |
| | | FNFNGLTGTGVLTESNKKFLPFQQFGRDIDDTTDAVRDPQTLEILDITPCS |
| 163 | SARS-CoV-2 | MSTMRLLTLALLFSCSVARAACRVQPTESIVRFPNITNLCPFGEVFNATRFASVYA |
| | B.1.1.7 Spike | WNRKRISNCVADYSVLYNSASFSTFKCYGVSPTKLNDLCFTNVYADSFVIRGDEVR |
| | RBD-Fc | QIAPGQTGKIADYNYKLPDDFTGCVIAWNSNNLDSKVGGNYNYLYRLFRKSNLKPF |
| | | ERDISTEIYQAGSTPCNGVEGFNCYFPLQSYGFQPTYGVGYQPYRVVVLSFELLH |
| | | APATVCGPKKSTNLVKNKCVNFNFNGLTGTGVLTESNKKFLPFQQFGRDIDDTTD |
| | | AVRDPQTLEILDITPCSGSSTMVRSSKPTCPPPELLGGPSVFIFPPKPKDTLMISRT |
| | | PEVTCVVVDVSQDDPEVQFTWYINNEQVRTARPPLREQQFNSTIRVVSTLPIAHQD |
| | | WLRGKEFKCKVHNKALPAPIEKTISKARGQPLEPKVYTMGPPREELSSRSVSLTC |
| | | MINGFYPSDISVEWEKNGKAEDNYKTTPAVLDSDGSYFLYSKLSVPTSEWQRGDV |
| | | FTCSVMHEALHNHYTQKSISRSPGK |
| 164 | RBD-SD1 | RVQPTESIVRFPNITNLCPFGEVFNATRFASVYAWNRKRISNCVADYSVLYNSASF |
| | region of | STFKCYGVSPTKLNDLCFTNVYADSFVIRGDEVRQIAPGQTGNIADYNYKLPDDFT |
| | SARS-CoV-2 | GCVIAWNSNNLDSKVGGNYNYLYRLFRKSNLKPFERDISTEIYQAGSTPCNGVKG |
| | B.1.351 Spike | FNCYFPLQSYGFQPTYGVGYQPYRVVVLSFELLHAPATVCGPKKSTNLVKNKCVN |
| | | FNFNGLTGTGVLTESNKKFLPFQQFGRDIADTTDAVRDPQTLEILDITPCS |
| 165 | SARS-CoV-2 | MSTMRLLTLALLFSCSVARAACRVQPTESIVRFPNITNLCPFGEVFNATRFASVYA |
| | B.1.351 Spike | WNRKRISNCVADYSVLYNSASFSTFKCYGVSPTKLNDLCFTNVYADSFVIRGDEVR |
| | RBD-Fc | QIAPGQTGNIADYNYKLPDDFTGCVIAWNSNNLDSKVGGNYNYLYRLFRKSNLKPF |
| | | ERDISTEIYQAGSTPCNGVKGFNCYFPLQSYGFQPTYGVGYQPYRVVVLSFELLH |
| | | APATVCGPKKSTNLVKNKCVNFNFNGLTGTGVLTESNKKFLPFQQFGRDIADTTD |
| | | AVRDPQTLEILDITPCSGSSTMVRSSKPTCPPPELLGGPSVFIFPPKPKDTLMISRT |
| | | PEVTCVVVDVSQDDPEVQFTWYINNEQVRTARPPLREQQFNSTIRVVSTLPIAHQD |
| | | WLRGKEFKCKVHNKALPAPIEKTISKARGQPLEPKVYTMGPPREELSSRSVSLTC |
| | | MINGFYPSDISVEWEKNGKAEDNYKTTPAVLDSDGSYFLYSKLSVPTSEWQRGDV |
| | | FTCSVMHEALHNHYTQKSISRSPGK |

### FIGURES

The invention is demonstrated by way of the example through the figures disclosed herein. The figures provided represent particular, non-limiting embodiments and are not intended to limit the scope of the invention.

Short description of the figures:

FIG. 1: Illustration of the cell sorting strategy

FIG. 2: SARS-CoV-2 RBD ELISA

FIG. 3: ACE2 competition assay

FIG. 4: Plaque Reduction Neutralization Assay

FIG. 5: Screening for off-target reactivity

FIG. 6: Identification and characterization of potent SARS-CoV-2 neutralizing mAbs

FIG. 7. SARS-CoV-2-S1 serum IgG response from COVID-19 patients, flow cytometry gating and characteristics of immunoglobulin sequences

FIG. 8: Binding kinetic measurements of mAbs to RBD.

FIG. 9: Bind epitope Characterization of selected mAbs.

FIG. 10. SARS-CoV-2 neutralizing antibodies can bind to murine tissue

FIG. 11: Prophylactic and therapeutic application of mAb CV07-209 in a COVID-19 hamster model

FIG. 12: Histopathological analysis of hamsters after SARS-CoV-2 infection

FIG. 13: ELISA with SARS-CoV-2 RBD of variants of concern

FIG. 14: Plaque Reduction Neutralization Assay using SARS-CoV-2 variants

### DETAILED DESCRIPTION OF THE FIGURES

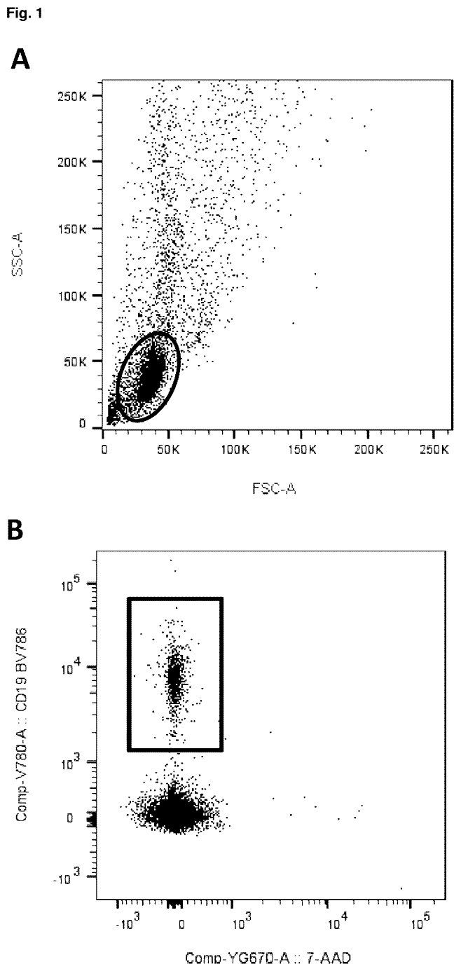

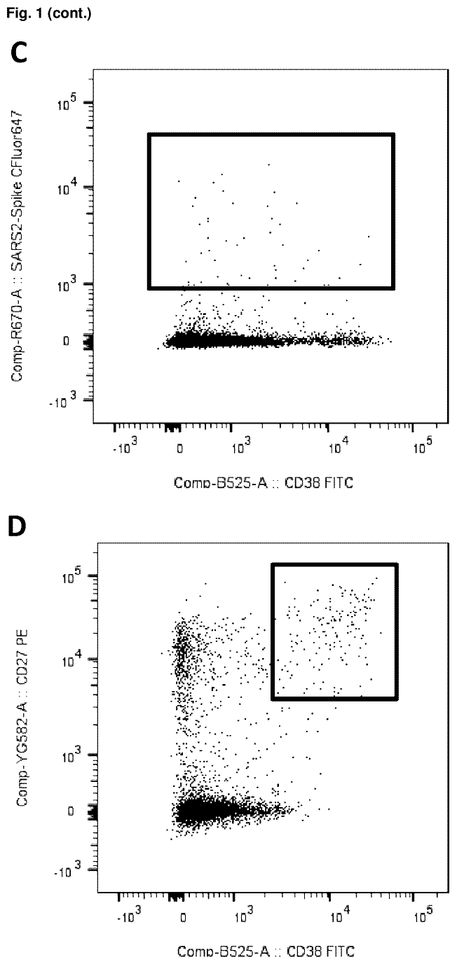

### FIG. 1: Illustration of the Cell Sorting Strategy

For generation of recombinant human monoclonal antibodies from convalescent COVID-19 patients single cells from two cell populations were isolated using flow cytometry by sequential selections (A) on lymphocytes by typical size and granularity; (B) on viable cells from B cell lineage (AAD-negative, CD19+); and then either (C) on antigen-enriched memory B cells (labeled with S1 SARS-CoV-2, after pre-selection on CD27+ memory B cells); or (D) plasma blasts (CD27+ and CD38+).

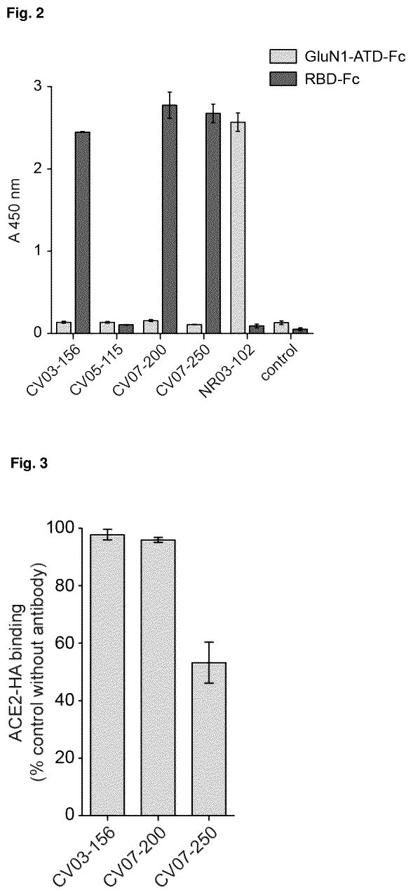

### FIG. 2: SARS-CoV-2 RBD ELISA

Cell culture supernatant containing S1 SARS-CoV-2 reactive human monoclonal antibodies from convalescent COVID-19 patients were screened for binding to receptor binding domain (RBD) of S1 SARS-CoV-2 in an ELISA based assay. Cell culture supernatants were diluted 1:5 in 0.4% BSA/PBS-T, applied to RBD-Fc for 2 hours at room temperature and detected using horseradish peroxidase (HRP)-conjugated anti-human IgG antibody and the HRP substrate ultraTMB. Data for four S1 SARS-CoV-2 reactive monoclonal antibodies is shown as mean+/−SD of two wells from one experiment.

### FIG. 3: ACE2 Competition Assay

Human monoclonal SARS-CoV-2 RBD reactive antibodies were screened for inhibition of the binding of human Angiotensin-converting enzyme 2 (ACE2) to RBD in an ELISA based approach. The antibodies were diluted to 0.5 μg/ml in DMEM/FBS/PBS-T and applied to RBD-Fc for 15 minutes at room temperature. After brief washing with PBS-T, ACE2-HA was applied in DMEM/FBS/PBS-T for 1 hour at room temperature. Data for three RBD monoclonal antibodies is shown as mean+/−SD from two independent experiments.

### FIG. 4: Plaque Reduction Neutralization Assay

Human monoclonal SARS-CoV-2 antibodies strongly binding to RBD and competing with RBD-binding to human ACE2 were screened for neutralization of virus induced pathological effects on human VeroE6-cells in a plaque reduction neutralization assay (PRNT). Monoclonal antibodies in serial dilutions were pre-incubated with SARS-CoV-2 virus before addition to cultured VeroE6-cells. After incubation at 37° C. and 5% CO2 for 3 days plaque building was quantified in relation to no-antibody condition. Data is shown from two independent dilutions as mean+/−SD.

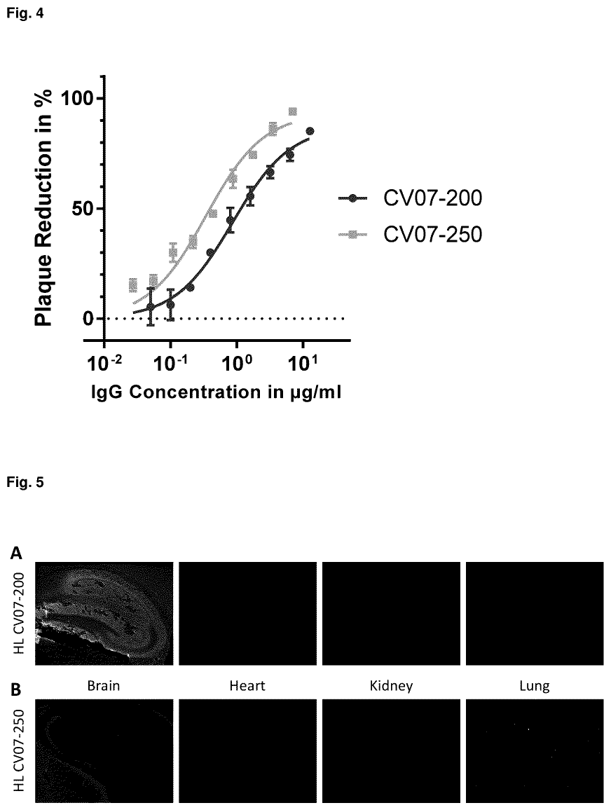

### FIG. 5: Screening for Off-Target Reactivity

SARS-CoV-2 monoclonal antibodies were screened for binding to mammalian tissue. Human antibodies were stained on 20 μm cryo-sections of murine tissue from brain, heart, kidney and lung, followed by incubation with fluorophore-tagged anti-human secondary antibody. (A) Antibody HL CV07-200 showed intensive staining signal to murine brain, most pronounced in hippocampus (white), but did not stain heart, kidney or lung tissue. (B) Antibody HL CV07-250 did not react with murine tissue in indicated organs.

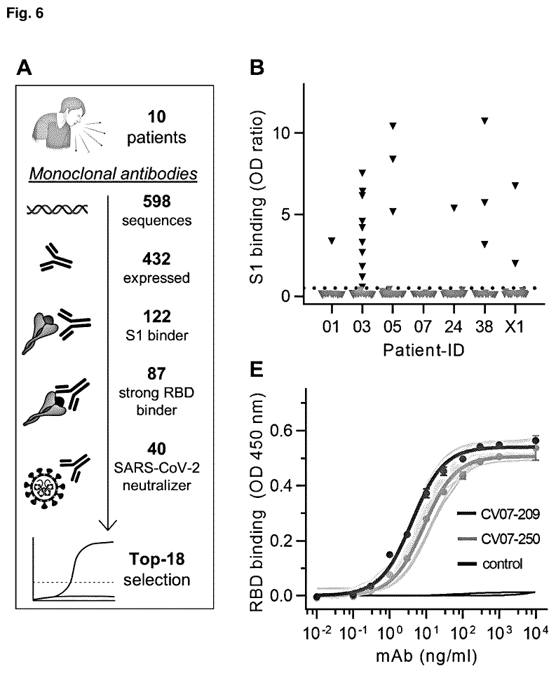

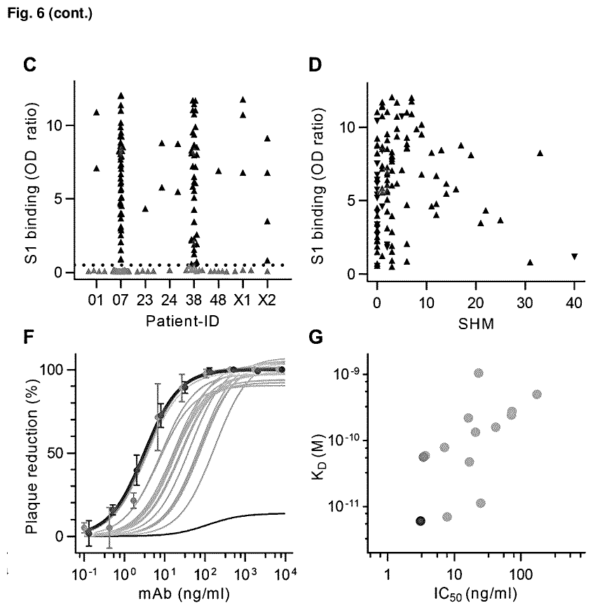

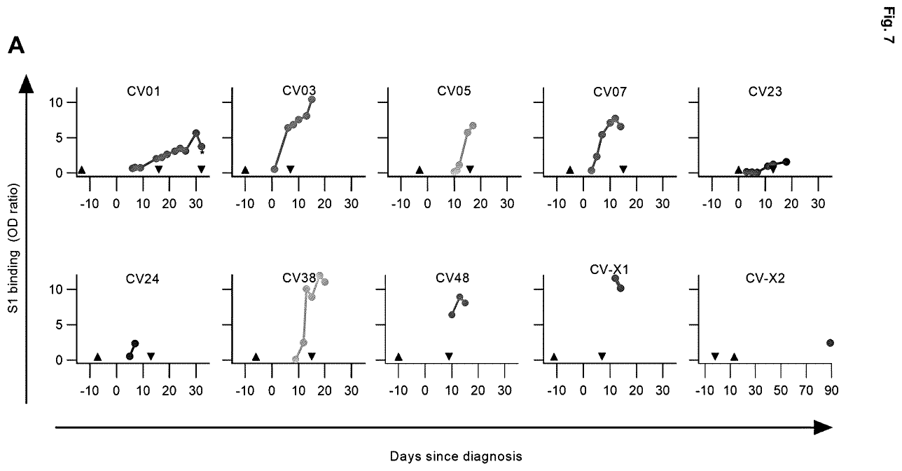

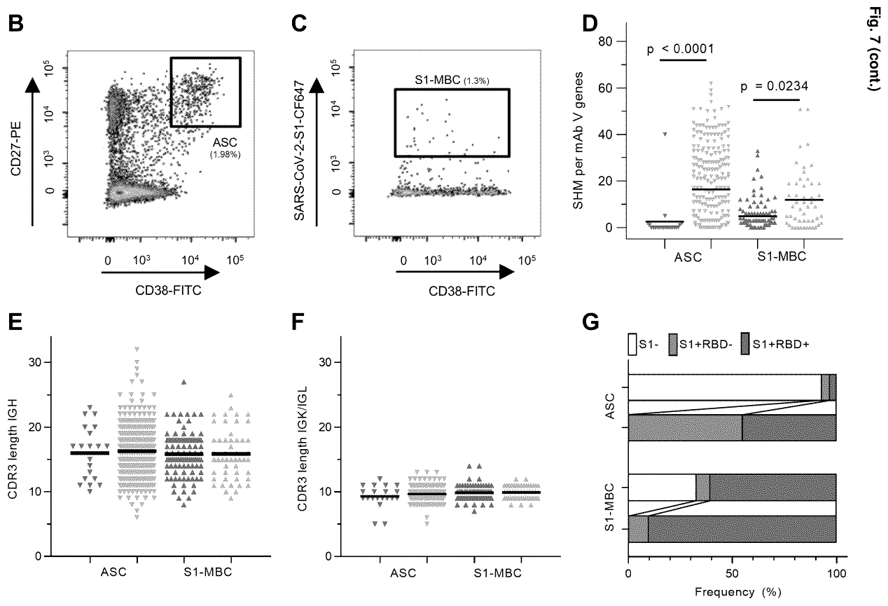

### FIG. 6: Identification and Characterization of Potent SARS-CoV-2 Neutralizing mAbs

(A) Diagram depicting the strategy for isolation of 18 potently neutralizing mAbs (Top-18). (B) Normalized binding to S1 of SARS-CoV-2 for mAbs isolated from antibody secreting cells (▾; blue=S1-binding, grey=not S1-binding). (OD=optical density in ELISA). (C) Normalized binding to S1 of SARS-CoV-2 for mAbs isolated from S1-stained memory B cells (▴; colors like in (B)). (D) S1-binding plotted against the number of somatic hypermutations (SHM) for all S1-reactive mAbs. (E) Concentration-dependent binding of Top-18 SARS-CoV-2 mAbs to the RBD of S1 (mean±SD from two wells of one experiment). (F) Concentration-dependent neutralization of authentic SARS-CoV-2 plaque formation by Top-18 mAbs (mean±SD from two independent measurements). (G) Apparent affinities of mAbs to RBDs (KD determined by surface plasmon resonance) plotted against IC50 of authentic SARS-CoV-2 neutralization.

### FIG. 7: SARS-CoV-2-S1 Serum IgG Response From COVID-19 Patients, Flow Cytometry Gating and Characteristics of Immunoglobulin Sequences

(A) Serum IgG response determined as the normalized optical density (OD) in a SARS-CoV-2-S1 ELISA in relation to the time point of diagnosis defined by the first positive qPCR test. Upward arrowhead denotes the appearance of first symptoms. Downward arrowhead denotes the PBMC isolation. From patient CV01, PBMC samples were isolated at two time points as indicated by the second downward arrow with an asterisk (*). (B-C) A representative flow cytometry plot from patient CV38 indicating gating on (B) CD19⁺CD27⁺antibody-secreting cells (ASC) and (C) SARS-CoV-2-S1-stained memory B cells (S1-MBC). Cells were pre-gated on live CD19⁺ B cells. (D) Comparison of somatic hypermutation (SHM) count within immunoglobulin V genes combined from heavy and light chains of S1-reactive (S1+, blue) and non-S1-reactive (S1−, grey) mAbs. Statistical significance was determined using a Kruskal-Wallis test with Dunn's multiple comparison test. (ASC: n=20 S1+, n=260 S1−; S1-MBC: n=102 S1+, n=50 S1−, n-values represent number of mAbs). All expressed mAbs are displayed. Each triangle represents one mAb, isolated from an ASC (▾) or a S1-MBC (▴). Bars indicate mean. (E-F) Length comparison of complementarity-determining region (CDR) 3 amino acid sequences between S1+ and S1− mAbs within (E) heavy and (F) light chains. Bars indicate mean. Symbols and colors have the same meaning as in (D). (G) Frequency of RBD-binder (S1+RBD+) and non-RBD-binder (S1+RBD−) relative to all expressed mAbs (upper lanes) and relative to S1+ mAbs (lower lanes).

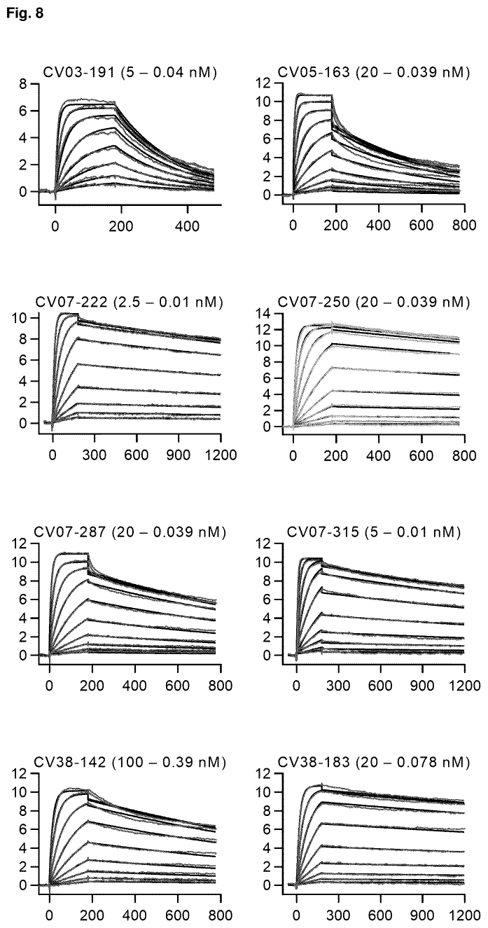

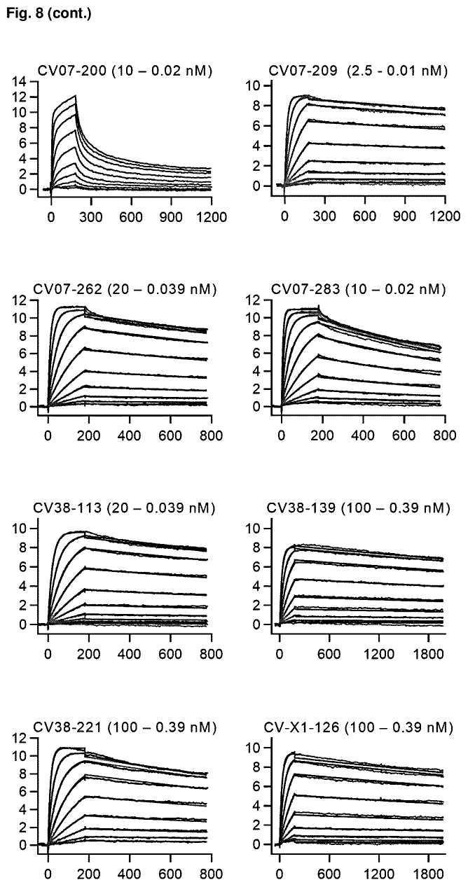

### FIG. 8: Binding Kinetic Measurements of mAbs to RBD

Binding kinetics of mAbs to RBD were modeled (black) from multi-cycle surface plasmon resonance (SPR) measurements (blue, purple, orange). Fitted monovalent analyte model is shown. For CV07-200, neither a bivalent nor a monovalent analyte model described the data accurately (no model is shown). Three out of the 18 selected mAbs for detailed characterization (Top-18) were not analyzed using multi-cycle-kinetics: CV07-270 was excluded as it interacted with the anti-mouse IgG reference surface on initial qualitative measurements. CV07-255 and CV-X2-106 were not analyzed since they showed biphasic binding kinetics and relatively fast dissociation rates in initial qualitative measurements. Non-neutralizing CV03-191, a mAb not included in the Top-18 mAbs, was included in the multicycle experiments as it has the same clonotype as strongly neutralizing CV07-209 (Figure S4C). All measurements are performed by using a serial 2-fold dilution of mAbs on reversibly immobilized SARS-CoV-2-S1 RBD-mFc.

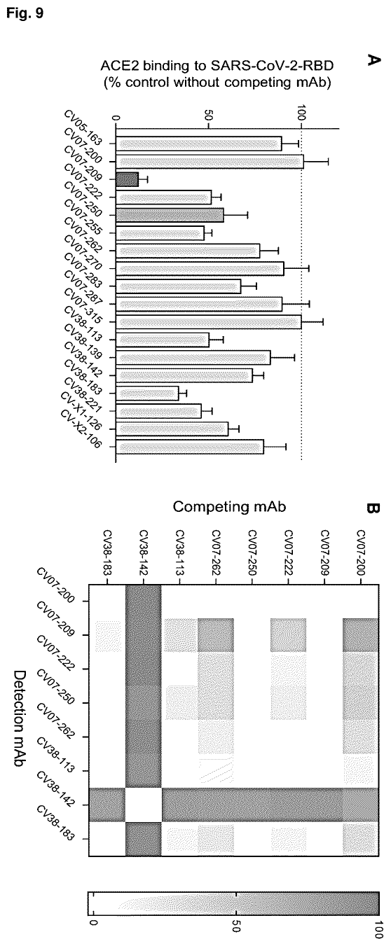

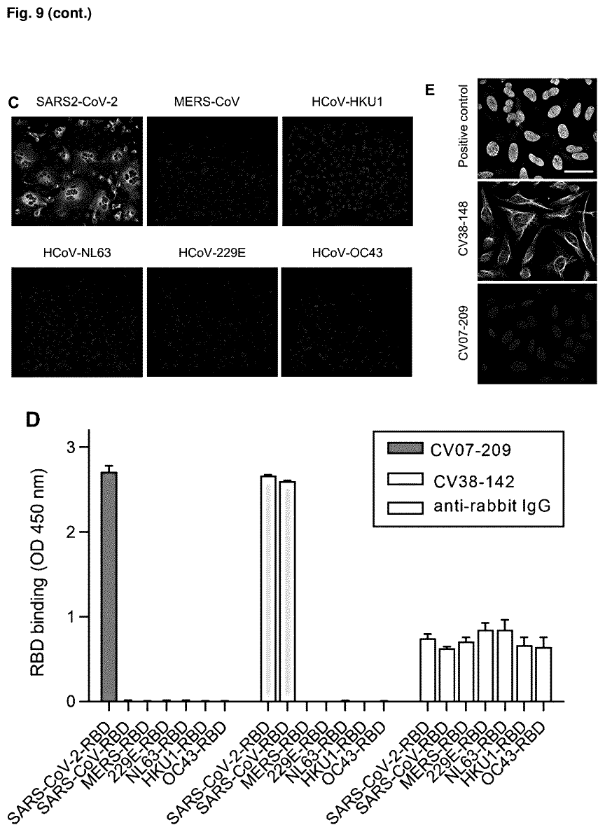

### FIG. 9: Epitope Characterization of Selected mAbs

(A) Competition for RBD binding between Top-18 mAbs and ACE2. ELISA-based measurements of human ACE2 binding to SARS-CoV-2 RBD after pre-incubation with the indicated neutralizing mAbs. Values are shown relative to antibody-free condition as mean±SD from three independent measurements. (B) Competition for RBD binding between combinations of potent neutralizing mAbs is illustrated as a heat map. Shades of green indicate the degree of competition for RBD binding of detection mAb in presence of 100-fold excess of competing mAb relative to non-competition conditions. Green squares indicate no competition. Values are shown as mean of two independent experiments. (C) Representative immunofluorescence staining on VeroB4 cells overexpressing spike protein of indicated coronavirus with SARS-CoV-2 mAb CV07-209 at 5 μg/ml. For all other 17 of the selected 18 mAbs (Top-18, Table S3), similar results were obtained. (D) Binding of indicated mAbs to fusion proteins containing the RBD of indicated coronaviruses and the constant region of rabbit IgG revealed by ELISA. For all other Top-18 mAbs, similar results were obtained as for CV07-209. Values indicate mean±SD from two wells of one experiment. (E) Representative HEp-2 cell staining with a commercial anti-nuclear antibody as positive control revealed nuclear binding (top). S1-reactive non-neutralizing mAb CV38-148 exhibited cytoplasmatic binding (middle). Neutralizing mAb CV07-209 showed no binding (bottom). All mAbs selected for detailed characterization (Top-18, Table S3) revealed similar results like CV07-209 when used at 50 μg/ml. Representative scale bar: 25 μm.

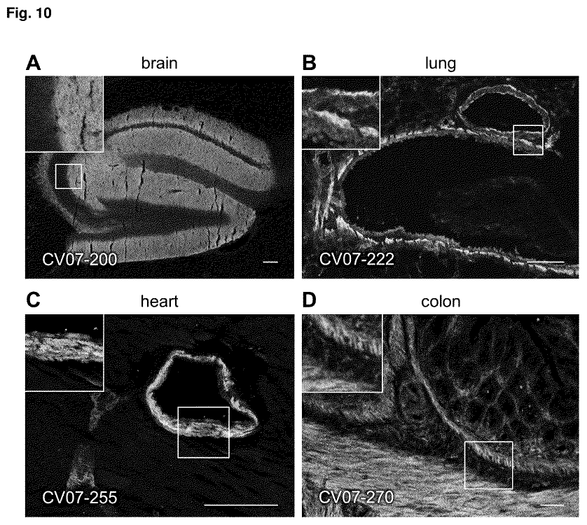

### FIG. 10: SARS-CoV-2 Neutralizing Antibodies can Bind to Murine Tissue

Immunofluorescence staining of SARS-CoV-2 mAbs (green) on murine organ sections showed specific binding to distinct anatomical structures, including (A) staining of hippocampal neuropil with CV07-200 (cell nuclei depicted in blue), (B) staining of bronchial walls with CV07-222, (C) staining of vascular walls with CV07-255, and (D) staining of intestinal walls with CV07-270. Smooth muscle tissue in (B-D) was co-stained with a commercial smooth muscle actin antibody (red). Scale bars: 100 μm. See also Table 2.

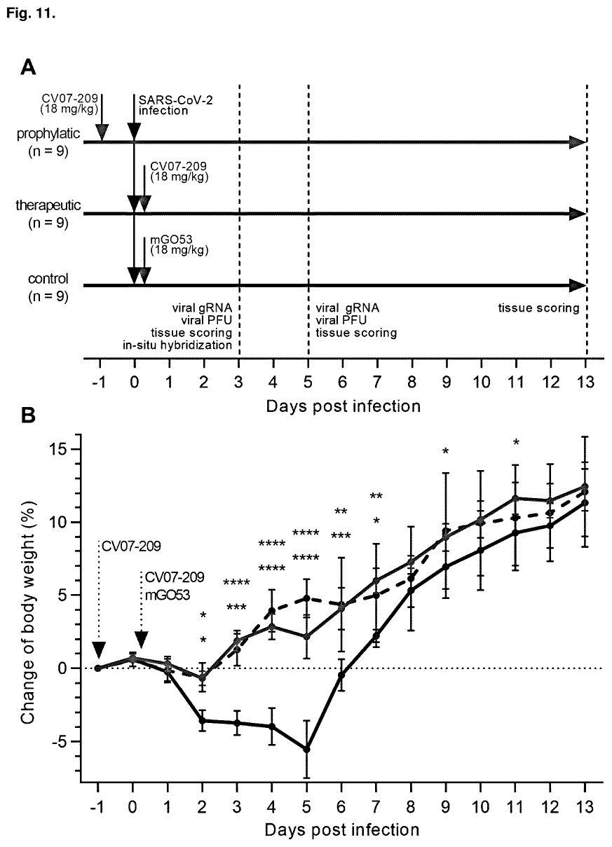

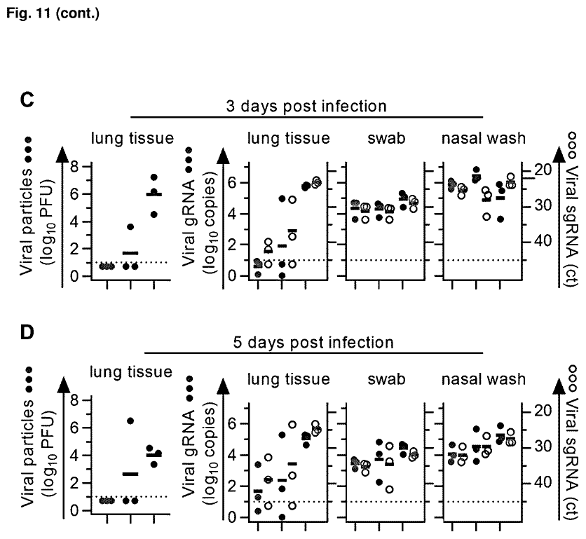

### FIG. 11: Prophylactic and Therapeutic Application of mAb CV07-209 in a COVID-19 Hamster Model

(A) Schematic overview of the animal experiment. (B) Body weight of hamsters after virus challenge and prophylactic (pink) or therapeutic (blue) application of SARS-CoV-2 neutralizing mAb CV07-209 or control antibody (mean±SEM from n=9 animals per group from day −1 to 3, n=6 from days 4 to 5; n=3 from days 6 to 13; mixed-effects model with posthoc Dunnett's multiple tests in comparison to control group; significance levels shown as *(p<0.05), **(p<0.01), ***(p<0.001), ****(p<0.0001), or not shown when not significant). (C-D) Left: Quantification of plaque forming units (PFU) from lung homogenates. Right: Quantification of genomic SARS-CoV-2 RNA (gRNA) as copies per 10⁵ cellular transcripts (left y-axis, filled circles) and cycle threshold (ct) of subgenomic SARS-CoV-2 RNA (sgRNA) detection (right y-axis, unfilled circles) from samples and timepoints as indicated. Values for PFU were set to 5 when not detected, gRNA copies below 1 were set to 1 and ct of sgRNA to 46 when not detected. Bars indicate mean. Dotted lines represent detection threshold.

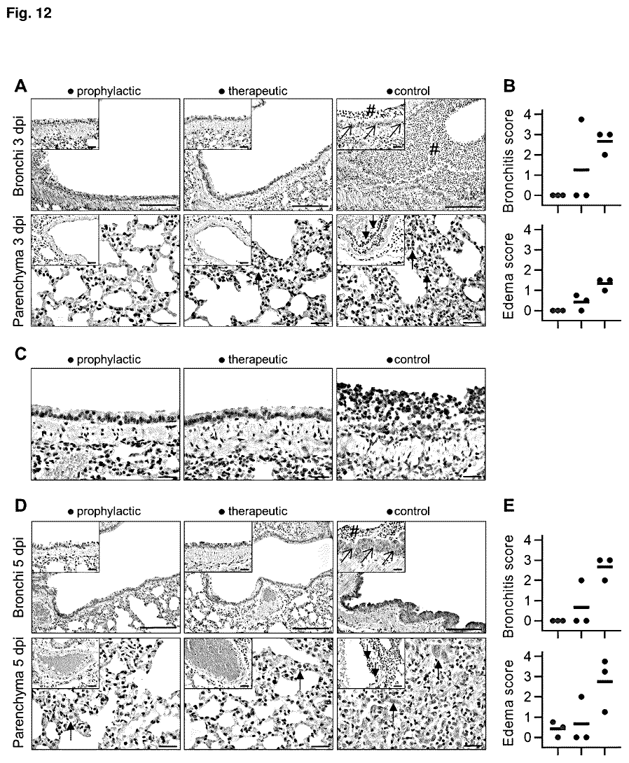

### FIG. 12: Histopathological Analysis of Hamsters After SARS-CoV-2 Infection

(A) Histopathology of representative haematoxylin and eosin stained, paraffin-embedded bronchi with inserted epithelium (upper row) and lung parenchyma with inserted blood vessels (lower row) at 3 dpi. Severe suppurative bronchitis with immune cell infiltration (hash) is apparent only in the control-treated animals with necrosis of bronchial epithelial cells (diagonal arrows). Necro-suppurative interstitial pneumonia (upward arrows) with endothelialitis (downward arrows) is prominent in control-treated animals. Scale bars: 200 μm in bronchus overview, 50 μm in all others. (B) Bronchitis and edema score at 3 dpi. Bars indicate mean. (C) Detection of viral RNA (red) using in situ hybridization of representative bronchial epithelium present only in the control group. Scale bars: 50 μm. (D) Histopathology of representative lung sections from comparable areas as in (A) at 5 dpi. Staining of bronchi of control-treated animals showed a marked bronchial hyperplasia with hyperplasia of epithelial cells (diagonal arrow) and still existing bronchitis (hash), absent in all prophylactically treated and in ⅔ therapeutically treated animals (upper row). Lung parenchyma staining of control-treated animals showed severe interstitial pneumonia with marked type II alveolar epithelial cell hyperplasia and endothelialitis (insets, downward arrows). Compared to control-treated animals, prophylactically treated animals showed only mild signs of interstitial pneumonia with mild type II alveolar epithelial cell hyperplasia (upward arrow), whereas therapeutically treated animals showed a more heterogeneous picture with ⅓ showing no signs of lung pathology, ⅓ animal showing only mild signs of interstitial pneumonia, and ⅓ animal showing a moderate multifocal interstitial pneumonia. Scale bars: 200 μm in bronchus overview, 50 μm in all others. (E) Bronchitis and edema score at 5 dpi. Bars indicate mean.

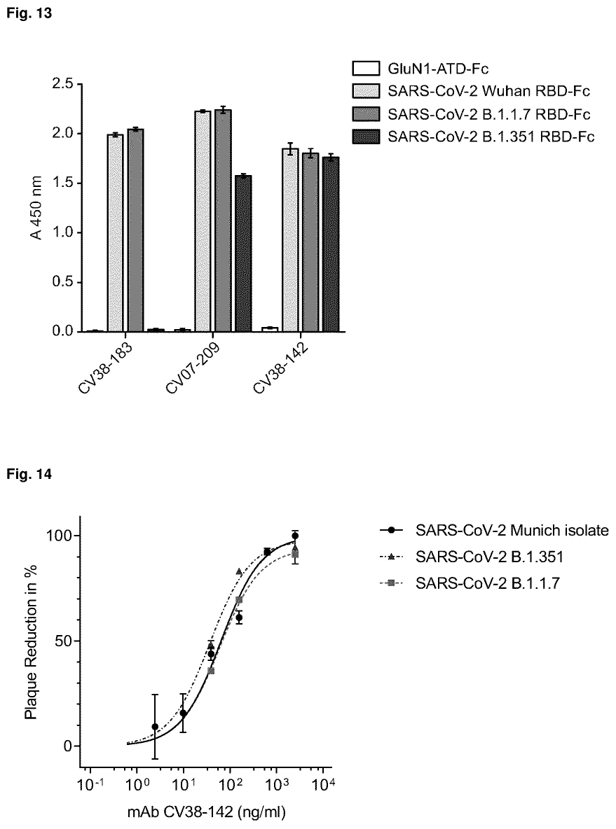

### FIG. 13: ELISA With SARS-CoV-2 RBD of Variants of Concern

Human monoclonal antibodies from convalescent COVID-19 patients reactive to the Wuhan SARS-CoV-2 RBD were tested for binding to the RBD of SARS-CoV-2 lineages B.1.1.7 and B.1.351 in an ELISA based assay. The antibodies were diluted to 0.1 μg/ml in DMEM/FBS/PBS-T, applied for 15 minutes at room temperature and detected using horseradish peroxidase (HRP)-conjugated anti-human IgG antibody and the HRP substrate ultraTMB. Data is shown as mean+/−SD of two wells from one experiment after subtraction of the signal generated by the anti-human IgG antibody alone.

### FIG. 14: Plaque Reduction Neutralization Assay Using SARS-CoV-2 Variants

Human monoclonal antibodies strongly neutralizing SARS-CoV-2 were screened for neutralizing breadth against viral variants of concern in a plaque reduction neutralization assay (PRNT) on human VeroE6-cells in. Neutralization data of human monoclonal antibody CV38-142 is shown. Serial dilutions of CV38-142 were pre-incubated with SARS-CoV-2 virus from isolate/lineage as indicated before addition to cultured VeroE6-cells. After incubation at 37° C. and 5% CO2 for 3 days plaque building was quantified in relation to no-antibody condition. Data is shown from two independent dilutions as mean+/−SD.

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

The examples below present the isolation and characterization of antibodies suitable for the treatment and prophylaxis of Coronavirus-mediated disease, such as COVID-19, that were isolated from convalescent COVID-19 patients. In preferred embodiments, the antibodies block the interaction of SARS-CoV-2 with the human host cells by influencing the binding between the Spike protein RBD and ACE2. In preferred embodiments, the antibodies block infection of endothelial cells by SARS-CoV-2.

The isolation of these antibodies and their corresponding binding properties distinguish the monoclonal antibodies characterized herein from developments of other research groups (for example Ju 2020). Described in more detail below is:
Flow cytometric single-cell sorting of enriched B-cells that bind (fluorophore) labelled Spike S1 protein synthesized in human cell lines,
Characterization of antibody-virus interaction by development of an RBD ELISA and an ELISA-based ACE2/RBD competitive assay,
Testing of antibodies in a neutralization assay to predict therapeutic effectiveness in preventing infection of human endothelial cells,
Screening for cross-reactivity to mammalian tissue in order to select out antibodies that exhibit an unwanted autoreactivity,
Testing biophysical and functional properties of the antibodies, such as affinity to the target antigen,
Testing selected antibodies in an in vivo animal model of SARS coronavirus associated disease, and
Testing binding properties of the antibodies against SARS-CoV-2 variants.

Examples 1-5 represent an initial assessment and characterization of the inventive antibodies. The following Examples represent a further, more detailed experimental assessment of the inventive antibodies.

### Example 1: Generation of Recombinant Monoclonal Spike S1 Reactive Antibodies From Blood Samples of Convalescent COVID-19 Patients

The starting material for the recombinant production of antibodies was single B cells, whose genetic information was extracted and cloned into expression vectors.

For the cloning and production of antibodies from B cells we used a pipeline that was established in our research group (Kreye et al. 2016, Kornau et al. 2020). The special feature of the approach presented here for the generation of the SARS-CoV-2 antibodies is the use of a dual sorting strategy for the isolation of single B cells from the blood of patients with past COVID-19 infection:
1. Single-cell sorting of antibody-producing cells was carried out by selecting CD19+CD27+CD38+ cells in an unbiased selection (unbiased approach), and
2. Memory B cells were enriched by means of Spike protein synthesized in human cell lines (HEK293), which was coupled with a fluorophore (non-specific esterification). This protein enriches those B-cells that bind the virus on the surface via their B-cell receptors/antibodies. Thus, the number of SARS-CoV-2 reactive antibodies under investigation could be significantly increased. For the coupling we used the fluorophore CruzFluor-647, and for the viral spike protein we used human recombinant protein produced in human HEK cells, as this has the advantage of being as similar as possible to the protein found in human infections, thus leading to the most specific binding possible. FIG. 1 shows an illustration of the cell sorting strategy employed.

From single cell cDNA, recombinant monoclonal antibodies were generated following established protocols using a nested PCR strategy to amplify variable domains of immunoglobulin (Ig) heavy and light chain genes. For Ig sequence analysis, the customized Brain Antibody Sequence Evaluation (BASE) software (Reincke et al. 2019) was used. Functional Ig genes were cloned into respective expression vectors containing the constant Ig domains by Gibson assembly cloning. For mAb expression human embryonic kidney cells (HEK293T) were transiently transfected with matching Ig heavy and light chains. Cell culture supernatant containing mAb was harvested, Ig concentrations determined and then used for reactivity screenings and neutralization screening. For detailed characterization of mAb biophysical properties, supernatants were purified using Protein G Sepharose beads.

### Example 2: Spike-S1 and Spike-S1-RBD Reactivity Testing of Monoclonal Antibodies

In order to characterize the reactivity of the recombinantly produced antibodies, we performed a multi-step test. First, we identified all spike-S1-reactive antibodies in a routine ELISA in which binding to any domain of the viral S1 protein can be detected. In the second step we specifically selected those monoclonal antibodies whose binding is mediated by the viral spike-S1-RBD. For this purpose, we developed an ELISA based on an RBD-Fc fusion protein.

All recombinant mAbs were therefore first tested for S1-reactivity using routine ELISA testing with full length S1-CoV2-protein. S1-reactive mAbs were further characterized for binding to the RBD (receptor-binding domain) of protein S1 with a novel ELISA method.

A fusion protein construct containing the signal peptide of the NMDA receptor (NMDAR) subunit GluN1, the RBD-SD1 part of 2019-nCoV S (amino acids 319-591) and the constant region of rabbit IgG1 heavy chain (Fc) was generated, the protein termed RBD-Fc was expressed in HEK293 T cells and cell culture supernatants containing the secreted RBD-Fc were isolated three days later. The Fc moiety will likely induce dimerization and lend stability to the fusion protein. Based on RBD-Fc, an ELISA to test RBD-binding of mAbs was established.

Briefly, RBD-Fc and GluN1-ATD-Fc as a control were captured from cell culture supernatants onto 96-well plates via anti-rabbit IgG antibodies. Human mAbs were applied and bound antibody detected using horseradish peroxidase (HRP)-conjugated anti-human IgG antibody and the HRP substrate ultraTMB.

FIG. 2 shows a screening example for RBD reactivity by a panel of human monoclonal antibodies derived from B cells or antibody-secreting cells of COVID-19 patients. Each antibody was tested in parallel for binding to RBD-Fc and to the control protein GluN1-ATD-Fc. Three of the antibodies selectively recognized RBD, while a known anti-NMDAR autoantibody (NR03-102) recognized only GluN1 (positive control). The RBD binding test shown here identifies antibodies that bind the Spike RBD and may block the cellular invasion of SARS-CoV-2. It can also be used to compare the relative strength of RBD binding of antibodies by applying the antibodies at different concentrations.

As can be observed from FIG. 2, not all isolated antibodies that bound to the Spike S1 protein also bound to the RBD in this assay. These antibodies were removed from further analysis.

### Example 3: Antibody-Mediated Blocking of the Interaction of SARS-CoV-2 S RBD With ACE2

In the following step we investigated whether and to what extent the RBD antibodies inhibit the interaction between SARS-CoV-2 S and its cellular receptor ACE2. ACE2 is a high-affinity receptor of protein S, through which the entry of SARS-CoV-2 into the host cells is initiated.

To determine the effect of RBD-mAb-binding on the interaction between Angiotensin-converting enzyme 2 (ACE2) and SARS-CoV-2 RBD, the RBD-ELISA was further modified. A fusion protein containing the extracellular region of human ACE2 (amino acids 1-615), a His-tag and a hemagglutinin(HA)-tag was expressed in HEK293T cells and cell culture supernatants containing the secreted fusion protein (ACE2-HA) were harvested three days later.

To evaluate the ability of human antibodies to block the interaction between ACE2-HA and SARS-CoV-2 RBD, we incubated RBD-Fc captured on an ELISA plate with monoclonal antibodies recognizing RBD and subsequently with ACE2-HA-containing cell culture supernatants. Then, both human antibody binding and ACE2-HA binding were measured. A comparison of the ACE2-HA binding normalized to a control without human antibody revealed differential interference by the RBD-reactive antibodies.

The values for ACE2-HA binding were normalized to a control without human antibodies. Results are presented in FIG. 3 for a selection of tested antibodies.

The ACE2 competition assay allows the identification of monoclonal antibodies that compete with the interaction between ACE2 and RBD and are not displaced by ACE2 binding to RBD.

In addition, it demonstrates the efficiency of interference with ACE2 binding to RBD for individual antibodies. Antibodies that strongly inhibit the interaction between ACE2 and RBD have a high probability to prevent the cellular entry of SARS-CoV-2. Strong RBD binders that do not interfere with the RBD-ACE2 interaction can however prevent the virus from entering by other means. The use of a combination of such antibodies as therapeutic intervention for COVID-19 may benefit from additive effects targeting viral invasion.

### Example 4: Neutralization Assay

For further functional characterization, we selected recombinant monoclonal antibodies that cause strong inhibition of the RBD-ACE2 interaction and/or exhibit high affinity binding to RBD and examined them for their ability to neutralize the SARS-CoV-2 viruses.

For this purpose, human epithelial cells were infected with SARS-CoV-2 and the antibodies are added to the culture medium at a concentration of 0.001 to 10 μg/ml. After 3 days the cell culture plates are fixed with paraformaldehyde for 45 minutes and SARS-CoV-2 induced plaque formation is quantified and measured as a percentage plaque reduction compared to the antibody-free control condition.

Preliminary results are shown in the Table below (as a result of initial screening for multiple antibodies) and in FIG. 4 for the mAb HL CV07-250 (using a dilution series to determine efficacy more accurately). The initial screening (demonstrated in the Table below) employed cell supernatants after expression of the recombinant mAbs from cells hosting the corresponding coding DNA. For the more detailed analysis the mAb HL CV07-250 (FIG. 4) was purified from cell supernatants using Protein G Sepharose beads before being tested in the neutralization assay.

As can be seen from the initial screening results, the antibodies of the invention show promising inhibition of virus infection and inhibition of Spike-ACE2 interaction.

| Antibody | IC50 Neutralizing | | IC90 Neutralizing | | ACE2 |
|---|---|---|---|---|---|
| name | Assay | | Assay | | competition |
| HL CV07-250 | 50-500 | ng/ml | >500 | ng/μl | >40% |
| HL CV07-270 | 50-500 | ng/ml | about 500 | ng/μl | >20% |
| HL CV07-283 | about 50 | ng/ml | >500 | ng/μl | >30% |
| HK CV07-287 | 50-500 | ng/ml | >500 | ng/μl | >30% |
| HK CV05-163 | 50-500 | ng/ml | >500 | ng/μl | >10% |
| HK CV07-209 | <50 | ng/ml | 50-500 | ng/μl | >70% |
| HL CV07-222 | <50 | ng/ml | 50-500 | ng/μl | >30% |
| HL CV07-262 | <50 | ng/ml | about 500 | ng/μl | >10% |
| HK CV38-113 | about 50 | ng/ml | 50-500 | ng/μl | >40% |
| HL CV38-183 | <50 | ng/ml | 50-500 | ng/μl | >40% |
| HL CV07-255 | about 50 | ng/ml | n/a | | >20% |
| HL CV07-315 | about 50 | ng/ml | 50-500 | ng/ml | none |

| | | | | |
|---|---|---|---|---|
| HK CV38-139 | 50-500 | ng/ml | n/a | >70% |
| HK CV38-142 | about 50 | ng/ml | n/a | >10% |
| HK CVX1-126 | 50-500 | ng/ml | n/a | >30% |
| HK CV38-221 | 50-500 | ng/ml | n/a | >30% |
| HK CVX2-106 | about 500 | ng/ml | n/a | >40% |
| HK CV07-202 | 50-500 | ng/ml | n/a | >30% |
| HL CV07-267 | about 500 | ng/μl | n/a | >20% |

### Example 5: Reactivity Screening Against Murine Tissue

For reactivity screening against mammalian tissue, Sars-CoV-2 neutralizing mAbs were stained on murine tissue from lung, brain, heart, liver, kidney and gut as 20 μm unfixed cryo-sections mounted on glass slides. Tissue slices were thawed and rinsed with PBS, before blocking with blocking solution (PBS supplemented with 2% Bovine Serum Albumin and 5% Normal Goat Serum) for 1 hour at room temperature, before incubation of mAbs as undiluted cell supernatants or purified at 5 μg/ml overnight at 4° C. Section were then washed, Alexa Fluor 488-conjugated goat anti-human IgG applied for 2 hours at room temperature, then washed again and mounted, before examination under an inverted fluorescence microscope.

As can be seen in FIG. 5, some antibodies (HL CV07-200) exhibit unwanted cross-reactivity against mammalian tissue in vitro. Such antibodies are disregarded for further testing.

### Example 6: Further Characterization of Selected Antibodies

We systematically selected 18 strongly neutralizing mAbs out of 598 antibodies from 10 COVID-19 patients by characterization of their biophysical properties, authentic SARS-CoV-2 neutralization, and exclusion of off-target binding to murine tissue. Furthermore, we selected mAb CV07-209 by its in vitro efficacy and the absence of tissue-reactivity for in vivo evaluation. Systemic application of CV07-209 in a hamster model of SARS-CoV-2 infection led to profound reduction of clinical, paraclinical and histopathological COVID-19 pathology, thereby reflecting its potential for translational application in patients with COVID-19.

We first characterized the B cell response in COVID-19 using single-cell Ig gene sequencing of human mAbs (FIG. 6A). From ten COVID-19 patients with serum antibodies to the S1 subunit of the SARS-CoV-2 spike protein (FIG. 7A, Table 1), we isolated two populations of single cells from peripheral blood mononuclear cells with fluorescence-activated cell sorting (FACS): CD19+CD27+CD38+ antibody secreting cells (ASC) reflecting the overall humoral immune response and SARS-CoV-2-S1-labeled CD19+CD27+ memory B cells (S1-MBC) for characterization of antigen-specific responses (FIGS. 7B and 7C). We obtained 598 functional paired heavy and light chain Ig sequences. Of 432 recombinantly expressed mAbs, 122 were reactive to SARS-CoV-2-S1 (S1+), with a frequency of 0.0-18.2% (median 7.1%) within ASC and 16.7-84.1% (median 67.1%) within S1-MBC (FIGS. 6B and C). Binding to S1 did not depend on affinity maturation as measured by the number of SHM (FIG. 6D). Compared to mAbs not reactive to SARS-CoV-2-S1, S1+ mAbs had less SHM, but equal lengths for both their light and heavy chain complementarity-determining region 3 (CDR3) (FIGS. 7D, E and F). Within the ASC and S1-MBC population, 45.0% and 90.2% of S1+ mAbs bound the RBD, respectively (FIG. 7G).

TABLE 1
Description of patient cohort.

| | Patient ID | CV01 | CV03 | CV05 | CV07 | CV23 |
|---|---|---|---|---|---|---|
| General | (gender, age) | (m, 21) | (m, 46) | (m, 61) | (m, 32) | (f, 44) |
| Patient | Pre-existing | N | CVD, | N | N | atopic |
| history | conditions | | Crohn's | | | dermatitis, |
| | | | disease | | | hypthyreosis |
| | Smoking | N | N | N | Y | N |
| Symptoms | Pulmonary and | fever, | fever, | fever, cough, | fever, | fever |
| | general | cough, | cough, | dyspnea, | cough, | |
| | symptoms | emesis | dyspnea, | superinfection | dyspnea, | |
| | | | emesis | | respiratory | |
| | | | | | [symbol] | |
| | Neurological | loss of | loss of | N | loss of | n/a |
| | symptoms | smell, | smell, mood | | smell | |
| | | muscle | impairment, | | | |
| | | twitches | paresthesia | | | |
| Disease | Date symptom | 17 Feb. 2020 | 29 Feb. 2020 | 27 Feb. 2020 | 8 Mar. 2020 | 20 Mar. 2020 |
| course | onset | | | | | |
| | Days until | 13/13 | 10/11 | 3/13 | 5/6 | 0/0 |
| | diagnosis/ | | | | | |
| | hospitalization | | | | | |
| | SARS-CoV-2 | 2.0/3.8 | 6.4 | 5.7 | 6.6 | 1.2 |
| | serum IgG at | | | | | |
| | PBMC | | | | | |
| | isolation | | | | | |
| | Days in | 34/0 | 14/0 | 9/0 | 15/2 | 34/0 |
| | hospital/on | | | | | |
| | ICU | | | | | |
| | Oxygen | N/0 | N/0 | N/0 | Y/0 | N/0 |
| | demand/days | | | | | |
| | ventilation | | | | | |
| | Medication | N | N | AB, ACT | N | N |
| Follow Up | Outcome | discharged | discharged | discharged | discharged | discharged |

| | Patient ID | CV24 | CV38 | CV48 | CV-X1 | CV-X2 |
|---|---|---|---|---|---|---|
| General | (gender, age) | (m, 64) | (m, 70) | (m, 48) | (m, 46) | (m, 57) |
| Patient | Pre-existing | CVD | CVD | N | N | N |
| history | conditions | | | | | |
| | Smoking | N | Y (10 PY) | N | Y (7 PY) | N |
| Symptoms | Pulmonary | fever, | fever, | fever, | fever, | fever, |
| | and general | cough, | dyspnea, | cough, | cough, | cough, |
| | symptoms | dyspnea, | renal | dyspnea, | dyspnea, | epistaxis |
| | | emesis | failure, | diarrhea | respiratory | |
| | | | atrial | | failure, | |
| | Neurological | loss of | N | N | n/a | loss of |
| | symptoms | smell | | | | smell |
| Disease | Date | 13 Mar. 2020 | 12 Mar. 2020 | 14 Mar. 2020 | 15 Mar. 2020 | 18 Mar. 2020 |
| course | symptom | | | | | |
| | onset | | | | | |
| | Days until | 7/8 | 6/14 | 10/9 | 11/12 | 2/— |
| | diagnosis/ | | | | | |
| | hospitalization | | | | | |
| | SARS-CoV-2 | n/a | 8.9 | 6.4 | n/a | n/a |
| | serum IgG at | | | | | |
| | PBMC | | | | | |
| | isolation | | | | | |
| | Days in | 8/2 | 15/0 | 17/2 | 15/6 | 0/0 |
| | hospital/on | | | | | |
| | ICU | | | | | |
| | Oxygen | Y/0 | Y/0 | Y/0 | Y/3 | N/0 |
| | demand/ | | | | | |
| | days | | | | | |
| | ventilation | | | | | |
| | Medication | AB, ACT | AB, ACT, | AB, ACT, | AB, HCQ | N |
| | | | APT, | APT, HCQ | | |
| | | | Digitoxin | | | |
| Follow Up | Outcome | discharged | discharged | discharged | discharged | discharged |

PBMCs from ten COVID-19 patients were included in immunoglobulin repertoire studies. General information, patients' history, symptoms of SARS-CoV-2 infection, disease course and outcome are listed for each donor. SARS-CoV-2 IgG is given as a ratio of optical density from time point of PBMC isolation (±one day).
Abbreviations: m = male, f = female, Y = yes, N = no, n/a = not available, CVD = cardiovascular disease, PY = pack years, AB = antibiotics, ACT = anticoagulation therapy, APT = antiplatelet therapy, HCQ = hydroxychloroquine. [symbol]
indicates data missing or illegible when filed

### Example 7: Identification and Characterization of Potent SARS-CoV-2 Neutralizing mAbs

We next determined the mAbs with the highest capacity to neutralize SARS-CoV-2 in plaque reduction neutralization tests (PRNT) using authentic virus (Munich isolate 984). Of 87 mAbs strongly binding to RBD, 40 showed virus neutralization with a half-maximal inhibitory concentration (IC₅₀)≤250 ng/mL and were considered neutralizing antibodies (FIG. 6A), from which 18 (Top-18) were selected for further characterization (Table 2).

TABLE 2
Biophysical and functional characterization of the
18 most potent SARS-CoV-2 neutralizing mAbs.

| | EC₅₀ | Kₐ | K_{d} | K_{D} |
|---|---|---|---|---|
| mAb | (ng/ml) | (s⁻¹) | (M⁻¹s⁻¹) | (M) |
| CV05-163 | 3.8 | 8.48E+06 | 1.86E−03 | 2.20E−10 |
| CV07-200 | 9.0 | n/a | n/a | n/a |
| CV07-209 | 4.1 | 2.40E+07 | 1.44E−04 | 6.00E−12 |
| CV07-222 | 5.5 | 2.98E+07 | 2.07E−04 | 6.97E−12 |
| CV07-250 | 8.7 | 4.05E+06 | 2.28E−04 | 5.64E−11 |
| CV07-255 | 4.8 | n/a | n/a | n/a |
| CV07-262 | 12.1 | 4.27E+06 | 3.37E−04 | 7.90E−11 |
| CV07-270 | 14.2 | n/a | n/a | n/a |
| CV07-283 | 4.0 | 1.52E+07 | 7.24E−04 | 4.75E−11 |
| CV07-287 | 5.9 | 5.11E+06 | 8.14E−04 | 1.60E−10 |
| CV07-315 | 7.2 | 2.36E+07 | 2.62E−04 | 1.12E−11 |
| CV38-113 | 5.2 | 2.33E+06 | 3.12E−04 | 1.34E−10 |
| CV38-139 | 8.9 | 3.62E+05 | 1.00E−04 | 2.77E−10 |
| CV38-142 | 5.6 | 6.31E+05 | 6.60E−04 | 1.05E−09 |
| CV38-183 | 5.4 | 2.31E+06 | 1.37E−04 | 5.93E−11 |
| CV38-221 | 6.4 | 7.45E+05 | 3.71E−04 | 4.99E−10 |
| CV-X1-126 | 5.5 | 4.12E+05 | 9.98E−05 | 2.43E−10 |
| CV-X2-106 | 5.6 | n/a | n/a | n/a |

| | IC₅₀ | ACE2 binding | Tissue |
|---|---|---|---|
| mAb | (ng/ml) | reduction (%) | reactivity |
| CV05-163 | 16.3 | 9.6 | none |
| CV07-200 | 14.5 | none | B/L* |
| CV07-209 | 3.1 | 73.9 | none |
| CV07-222 | 7.8 | 36.9 | B/L/C |
| CV07-250 | 3.5 | 59.9 | none |
| CV07-255 | 14.5 | 51.6 | L/H/C/K |
| CV07-262 | 7.1 | 25.5 | none |
| CV07-270 | 82.3 | 7.5 | L/H*/C |
| CV07-283 | 16.9 | 38.0 | none |
| CV07-287 | 41.7 | 12.4 | none |
| CV07-315 | 24.9 | none | none |
| CV38-113 | 20.8 | 51.2 | none |
| CV38-139 | 73.2 | 14.9 | none |
| CV38-142 | 23.2 | 26.9 | none |
| CV38-183 | 3.7 | 65.9 | none |
| CV38-221 | 172.6 | 50.1 | none |
| CV-X1-126 | 71.7 | 44.2 | none |
| CV-X2-106 | 17.6 | 19.9 | none |

Abbreviations: EC₅₀ = half-maximal effective concentration, Kₐ = association rate, K_{d} = dissociation rate, K_{D} = equilibrium dissociation constant, IC₅₀ = half-maximal inhibitory concentration, B = brain, L = lung, H = heart, C = colon, K = kidney.
An asterisk (*) indicates weak binding.

The antibodies bound to RBD with a half-maximal effective concentration (EC₅₀) of 3.8-14.2 ng/mL (FIG. 6E) and an equilibrium dissociation constant (K_{D}) of 6.0 pM to 1.1 nM (FIG. 8, Table 2), thereby neutralizing SARS-CoV-2 with an IC₅₀ of 3.1-172 ng/mL (FIG. 6, Table 2). The antibody with the highest apparent affinity, CV07-209, was also the strongest neutralizer (FIG. 6).

We hypothesized that the differences in neutralizing capacity relate to different interactions with the ACE2 binding site. Indeed, the strongest neutralizing mAbs CV07-209 and CV07-250 reduced ACE2 binding to RBD to 12.4% and 58.3%, respectively. Other Top-18 mAbs including CV07-270 interfered only weakly with ACE2 binding (FIG. 9).

The spike proteins of SARS-CoV-2 and SARS-CoV share more than 70% amino acid sequence identity, whereas sequence identity between SARS-CoV-2 and MERS-CoV and other endemic coronaviruses is significantly lower. To analyze potential cross-reactivity of mAbs to other coronaviruses, we tested for binding of the Top-18 mAbs to the RBD of SARS-CoV, MERS-CoV, and the human endemic coronaviruses 229-E, NL63, HKU1 and OC43. CV38-142 detected the RBD of both SARS-CoV-2 and SARS-CoV, whereas no other mAb was cross-reactive to additional coronaviruses (FIGS. 9C and D). To further characterize the epitope of neutralizing mAbs, we performed ELISA-based epitope binning experiments using biotinylated antibodies. Co-applications of paired mAbs showed competition of most neutralizing antibodies for RBD binding (FIG. 9B). As an exception, SARS-CoV cross-reactive CV38-142 bound RBD irrespective of the presence of other mAbs, suggesting an independent and conserved target epitope (FIG. 9B).

### Example 8: Near-Germline SARS-CoV-2 Neutralizing Antibodies can Bind to Murine Tissue

Many SARS-CoV-2 neutralizing mAbs carry few SHM or are in germline configuration (FIG. 6D). Such antibodies close to germline might be reactive to more than one target. Prompted by the abundance of near-germline SARS-CoV-2 antibodies and to exclude potential side effects of mAb treatment, we next analyzed whether SARS-CoV-2 antibodies can bind to self-antigens.

Therefore, we tested the binding of S1-mAbs to unfixed murine tissues. Surprisingly, four of the Top-18 potent SARS-CoV-2 neutralizing mAbs showed anatomically distinct tissue reactivities (FIG. 10, Table 2). CV07-200 intensively stained brain sections in the hippocampal formation, olfactory bulb, cerebral cortex and basal ganglia (FIG. 10A). CV07-222 also bound to brain tissue, as well as to smooth muscle (FIG. 10B). CV07-255 and CV07-270 were reactive to smooth muscle from sections of lung, heart, kidney and colon, but not liver (FIGS. 10C and 10D, Table 2). None of the Top-18 mAbs bound to HEp-2 cells, cardiolipin or beta-2 microglobulin as established polyreactivity-related antigens (FIG. 9E).

The majority of our SARS-CoV-2 mAbs are close to germline configuration, supporting previous studies. Binding of some antibodies to HEp-2 cells was reported before, a finding we could confirm in our cohort. Given the increased probability of auto-reactivity of near-germline antibodies, we additionally examined for reactivity of SARS-CoV-2 mAbs with unfixed murine tissue, allowing the detection of reactivity to potential self-antigens in their natural conformation. Indeed, we found that a fraction of SARS-CoV-2 neutralizing antibodies also bound to brain, lung, heart, kidney or gut expressed epitopes. Such reactivity with host antigens should ideally be prevented by immunological tolerance mechanisms, but complete exclusion of such antibodies would generate “holes” in the antibody repertoire. In fact, HIV utilizes epitopes shared by its envelope and mammalian self-antigens, thus harnessing immunological tolerance to impair anti-HIV antibody responses and impeding successful vaccination. To defy viral escape in HIV, but similarly COVID-19, anergic strongly self-reactive B cells likely enter germinal centers and undergo clonal redemption to mutate away from self-reactivity, while retaining HIV or SARS-CoV-2 binding. Interestingly, longitudinal analysis of mAbs in COVID-19 showed that the number of SHM in SARS-CoV-2-neutralizing antibodies only marginally increased over time. This finding suggests that the self-reactivity observed in this study may not be limited to mAbs of the early humoral immune response in SARS-CoV-2 infections.

### Example 9: Crystal structures of Two mAbs Approaching the ACE2 Binding Site From Different Angles

Diffraction-quality crystals were obtained for SARS-CoV-2 RBD complexed with two individual neutralizing mAbs, CV07-250 and CV07-270, which have notable differences in the number of SHM, extent of ACE2 competition and binding to murine tissue. CV07-250 (IC₅₀=3.5 ng/ml) had 33 SHM (17/16 on heavy and light chain, respectively), strongly reduced ACE2 binding and showed no binding to murine tissue. In contrast, CV07-270 (IC₅₀=82.3 ng/ml) had only 2 SHM (2/0), did not reduce ACE2 binding in our assay, and showed binding to smooth muscle tissue. Using X-ray crystallography, the structures of CV07-250 and CV07-270 were determined in complex with SARS-CoV-2 RBD to resolutions of 2.55 and 2.70 Å, respectively.

The binding mode of CV07-250 to RBD is unusual in that it is dominated by the light chain, whereas in CV07-270, the heavy chain dominates as frequently found in other antibodies. The epitope of CV07-250 completely overlaps with the ACE2 binding site with a similar angle of approach as ACE2. In contrast, the CV07-270 epitope only partially overlaps with the ACE2 binding site and the antibody approaches the RBD from a different angle compared to CV07-250 and ACE2, explaining differences in ACE2 competition.

### Example 10: Prophylactic and Therapeutic mAbs in a COVID-19 Animal Model

Next, we selected mAb CV07-209 for evaluation of in vivo efficacy based on its high capacity to neutralize SARS-CoV-2 and the absence of reactivity to mammalian tissue. We used the hamster model of COVID-19, as it is characterized by rapid weight loss and severe lung pathology (Osterrieder et al., 2020). In this experimental set-up, hamsters were intranasally infected with authentic SARS-CoV-2. Nine hamsters per group received either a prophylactic application of CV07-209 24 hours before viral challenge, or a therapeutic application of CV07-209 or control antibody mGO53 two hours after viral challenge (FIG. 11A).

Hamsters under control mAb treatment lost 5.5±4.4% (mean±SD) of body weight, whereas those that received mAb CV07-209 as a therapeutic or prophylactic single dose gained 2.2±3.4% or 4.8±3.4% weight after 5 days post-infection (dpi), respectively. Mean body weights gradually converged in the animals followed up until 13 dpi, reflecting the recovery of control-treated hamsters from SARS-CoV-2 infection (FIG. 11B).

To investigate the presence of SARS-CoV-2 in the lungs, we measured functional SARS-CoV-2 particles from lung tissue homogenates. Plaque forming units were below the detection threshold for all animals in the prophylactic and in 2 of 3 in the treatment group at 3 and 5 dpi (FIGS. 11C and D). qPCR measurements of lung viral genomic RNA copies revealed a 4-5 and 3-4 log reduction at both time points in the prophylactic and therapeutic group, indicating a drastic decrease of SARS-CoV-2 particles in lungs after CV07-209 application. Reduced virus replication and cell infection was confirmed by lowered detection of subgenomic viral RNA (FIGS. 11C and D). However, genomic and subgenomic RNA levels from nasal washes and laryngeal swaps were similar between all groups, indicating virus replication in the upper airways (FIGS. 11C and D).

Additionally, we performed histopathological analyses of infected hamsters. As expected, all lungs from control-treated animals sacrificed at 3 dpi revealed typical histopathological signs of necro-suppurative pneumonia with suppurative bronchitis, necrosis of bronchial epithelial cells and endothelialitis (FIG. 12A). At 5 dpi, control-treated animals showed marked bronchial hyperplasia, severe interstitial pneumonia with marked type II alveolar epithelial cell hyperplasia and endothelialitis (FIG. 12D). In contrast, animals receiving prophylactic treatment with CV07-209 showed no signs of pneumonia, bronchitis, necrosis of bronchial epithelial cells, or endothelialitis at 3 dpi. A mild interstitial pneumonia with mild type II alveolar epithelial cell hyperplasia became apparent 5 dpi. Animals receiving therapeutic treatment with CV07-209 also showed a marked reduction of histopathological signs of COVID-19 pathology, although at both time points one out of three animals showed mild bronchopulmonary pathology with signs of interstitial pneumonia and endothelialitis. These qualitative findings were mirrored in the reduction of the bronchitis and edema scores (FIG. 12B, E).

To confirm the absence of viral particles under CV07-209 treatment, we performed in-situ hybridization of viral RNA at 3 dpi. No viral RNA was detectable in the prophylactic group, whereas all animals in the control group and one in the therapeutic group revealed intensive staining of viral RNA in proximity of bronchial epithelial cells (FIG. 12C). Taken together, these findings show that systemic application of SARS-CoV-2 neutralizing mAb CV07-209 protects hamsters from COVID-19 lung pathology and weight loss in both the prophylactic and the therapeutic setting.

We evaluated in detail the in vivo efficacy of the most potent neutralizing antibody CV07-209 in a Syrian hamster model of SARS-CoV-2 infection. This model is characterized by a severe phenotype including weight loss and distinct lung pathology. Our results demonstrated that prophylaxis and treatment with a single dose of CV07-209 not only led to clinical improvement as shown by the absence of weight loss, but also to markedly reduced lung pathology. While the findings confirm the efficacy of prophylactic mAb administration as described by other groups in mice, hamsters and rhesus macaques, our work also demonstrates the efficacy of post-exposure treatment in hamsters leading to viral clearance, clinical remission and prevention of lung injury. We provide detailed insights into the lung pathology of SARS-CoV-2 infected hamster at multiple times of the disease course including the regeneration phase. It complements two very recent demonstrations of a therapeutic effect of mAbs in a hamster model of COVID-19. These data expand the growing knowledge on post-exposure treatment from transgenic hACE2 mice and a mouse model using adenovector delivery of human ACE2 before viral challenge. Collectively, our results indicate that mAb treatment can be fine-tuned for exclusion of self-reactivity with mammalian tissues and that mAb administration can also be efficacious after the infection, which will be the prevailing setting in COVID-19 patients.

### Example 11: ELISA With SARS-CoV-2 RBD of Variants of Concern

Human monoclonal antibodies from convalescent COVID-19 patients reactive to the Wuhan SARS-CoV-2 RBD were tested for binding to the RBD of SARS-CoV-2 lineages B.1.1.7 and B.1.351 in an ELISA based assay. As can be seen from FIG. 13, all of CV38-183, CV07-209 and CV38-142 all bind to the Spike protein of the original Wuhan (AT) SARS-CoV-2 and B.1.1.7 in the ELISA assay. However, only CV07-209 and CV38-142 bind all three of the Wuhan, B.1.1.7 and B.1.351 variants. In particular, CV38-142 binds all three of the Wuhan, B.1.1.7 and B.1.351 variants in essentially the same manner.

### Example 12: Plaque Reduction Neutralization Assay Using SARS-CoV-2 Variants

Human monoclonal antibodies strongly neutralizing SARS-CoV-2 were screened for neutralizing breadth against viral variants of concern in a plaque reduction neutralization assay (PRNT) on human VeroE6-cells, using methods as described herein. Neutralization data of human monoclonal antibody CV38-142 is shown. Serial dilutions of CV38-142 were pre-incubated with SARS-CoV-2 virus from isolate/lineage as indicated before addition to cultured VeroE6-cells. As can be seen in FIG. 14, CV38-142 shows essentially the same effect in pRNT assays against all three of the Wuhan, B.1.1.7 and B.1.351 variants. CV38-142 therefore represents a preferred antibody with respect to its use in binding and/or neutralizing Spike protein of multiple SARS-CoV-2 variants.

### Example 13: Further Assessment of CV38-142

Since the spike protein is the major surface protein on coronaviruses, neutralizing antibodies are targeted towards the spike and many of these antibodies are able to prevent virus interaction with the host receptor, angiotensin-converting enzyme 2 (ACE2). Other inhibition mechanisms also seem to be possible and are being assessed for other subsets of antibodies. The receptor binding domain (RBD) of the spike protein is highly immunogenic and can induce highly specific and potent neutralizing antibodies (nAbs) against SARS-CoV-2 virus, as described herein. Many of these nAbs bind to the receptor binding site (RBS) on the RBD. However, the breadth of these nAbs is limited as the RBS shares relatively low sequence identity among sarbecoviruses; the RBS is only 48% conserved between SARS-CoV-2 and SARS-CoV compared to 73% for the complete RBD (84% identity for non-RBS regions of the RBD). The RBS is also prone to naturally occurring mutations, similar to the N-terminal domain (NTD), where insertions and deletions have also been found. Recent studies showed that many potent monoclonal neutralizing antibodies are subject to the antigenic drift or mutation on the RBD of the spike protein, as well as polyclonal sera from convalescent or vaccinated individuals.

Cross-neutralizing antibodies have been reported that bind to a highly conserved cryptic site in receptor binding domain (RBD) of the spike. Although the epitopes of these antibodies do not overlap with the ACE2 receptor binding site, some can sterically block ACE2 binding to the RBD or attenuate ACE2 binding affinity. Other RBD surfaces are also possible targets for cross-neutralizing antibodies, but are only moderately conserved within coronaviruses, although more so than the RBS.

High-resolution crystal structures of CV38-142 were determined in complex with both SARS-CoV RBD and SARS-CoV-2 RBD in combination with another cross-neutralizing antibody COVA1-16. This revealed that CV38-142 can be combined with cross-neutralizing antibodies to other epitopes to generate therapeutic cocktails that to protect against SARS-CoV-2 variants, escape mutants, and future zoonotic coronavirus epidemics. The information may also inform next generation vaccine and therapeutic design

As described herein, the antibody CV38-142 showed potent neutralization on authentic SARS-CoV-2 virus (Munich isolate 984) and is able to cross-react with SARS-CoV. CV38-142 is an IGHV5-51-encoded antibody with little somatic hypermutation (only four mutations in the amino-acid sequence). A biolayer interferometry (BLI) binding assay revealed that CV38-142 binds with high affinity not only to SARS-CoV-2 RBD (29 nM), but also SARS-CoV, RaTG13 and Guangdong pangolin coronavirus RBDs with roughly comparable affinity (36-99 nM). A pseudovirus neutralization assay showed that CV38-142 IgG neutralizes both SARS-CoV-2 and SARS-CoV with similar potency (3.5 and 1.4 μg/ml).

### Example 14: CV38-142 can be Combined With Other Antibodies

Recent reports on SARS-CoV-2 mutations in both human and mink populations give rise to concerns about viral escape from current vaccines and therapeutics in development. However, antibody cocktails that bind to distinct epitopes can increase neutralization breadth and may help prevent escape mutations. As described herein, CV38-142 does not compete for RBD binding with other potent antibodies in our sample set, such as CV07-200 (IGHV1-2), CV07-209 (IGHV3-11), CV07-222 (IGHV1-2), CV07-250 (IGHV1-18), CV07-262 (IGHV1-2), CV38-113 (IGHV3-53), and CV38-183 (IGHV3-53). CV38-142 can bind either SARS-CoV-2 RBD or spike protein at the same time in a sandwich assay as CC12.1 and COVA2-39, which are potent IGHV3-53 neutralizing antibodies from different cohorts. Since CC12.1, as well as COVA2-39 and CV07-250, bind to the RBS, these data suggest that CV38-142 can be combined with potent RBS antibodies in an antibody cocktail. Hence, it was tested whether CV38-142 could bind RBD at the same time as two other potent cross-neutralizing antibodies that target other sites on the RBD. A sandwich binding assay reveals that CV38-142 competes with S309 from a SARS patient, but is compatible with COVA1-16, a cross-neutralizing antibody to the CR3022 site isolated from a COVID-19 patient.

A cocktail consisting of different amounts and ratios of CV38-142 and COVA1-16 was assessed. The cocktail showed enhanced potency in a 2D neutralization matrix assay with SARS-CoV-2 and enhanced potency and improved efficacy with SARS-CoV pseudoviruses, demonstrating that CV38-142 is a promising candidate for pairing with cross-neutralizing antibodies to the CR3022 cryptic site. For example, 100% inhibition in the neutralization assay could be achieved with 1.6 μg/ml of each of CV138-142 and COVA1-16 with SARS-CoV-2 compared to >200 μg and 40 μg/ml for the individual antibodies, respectively. For SARS-CoV, the corresponding numbers were higher and required 200 μg/ml of each antibody to approach 100% inhibition, where 200 μg only achieved 77% and 28% neutralization, respectively, for each individual antibody. These changes in potency and efficacy show synergy between CV38-142 and COVA1-16. Synergistic neutralization effects have been analyzed in other viruses, including coronaviruses, and can be quantified by several algorithms using multiple synergistic models. Using the most up to date synergy model, a data analysis shows synergistic potency (α>1) between CV38-142 and COVA1-16 in two directions against both

SARS-CoV-2 and SARS-CoV pseudoviruses, indicating reciprocal synergy between CV38-142 and COVA1-16. Addition of COVA1-16 also improves the maximal efficacy of CV38-142 in neutralizing SARS-CoV as indicated by the positive synergistic efficacy score (β>0) as well as the neutralization matrix.

### Methods

### PBMC Collection and FACS Staining

Recombinant SARS-CoV-2-S1 protein produced in HEK cells (Creative Diagnostics, DAGC091) was covalently labeled using CruzFluor647 (Santa Cruz Biotechnology, sc-362620) according to the manufacturer's instructions.

Using fluorescence-activated cell sorting we sorted viable single cells from freshly isolated peripheral blood mononuclear cells (PBMCs as 7AAD⁻CD19⁺CD2⁺CD3⁺ antibody-secreting cells (ASCs) or SARS-CoV2-S1-enriched 7AAD⁻CD¹⁹⁺CD27⁺ memory B cells (MBCs) into 96-well PCR plates. Staining was performed on ice for 25 minutes in PBS with 2% FCS using the following antibodies: 7-AAD 1:400 (Thermo Fisher Scientific), CD19-BV786 1:20 (clone SJ25C1, BD Biosciences, 563326), CD27-PE 1:5 (clone M-T271, BD Biosciences, 555441), CD38-FITC 1:5 (clone HIT2, BD Biosciences, 560982), and SARS-CoV-S1-CF647 at 1 μg/ml for patients CV07, CV38, CV23, CV24, CV 38, CV48, CV-X1, CV-X2 and CV01 (second time point, fig. S1). The first patients (CV01 (first time point), CV03, and CV05) were stained with a divergent set of antibodies: CD19-PE 1:50 (clone HIB19, BioLegend, 302207), CD38-PEcy7 1:50 (clone HIT2, BioLegend, 303505), CD27-APC 1:50 (clone O323, BioLegend, 302809) and DAPI as viability dye.

### Generation of Recombinant Human Monoclonal Antibodies

Monoclonal antibodies were generated following established protocols with modifications as mentioned. We used a nested PCR strategy to amplify variable domains of immunoglobulin heavy and light chain genes from single cell cDNA and analyzed sequences with aBASE module of customized Brain Antibody Sequence Evaluation (BASE) software. Pairs of functional Ig genes were PCR-amplified using specific primers with Q5 Polymerase (NEB). PCR-product and linearized vector were assembled using Gibson cloning with HiFi DNA Assembly Master Mix (NEB). Cloning was considered successful when sequence identify >99.5% was given, verified by the cBASE module of BASE software. For mAb expression, human embryonic kidney cells (HEK293T) were transiently transfected with matching Ig heavy and light chains. Three days later mAb containing cell culture supernatant was harvested. Ig concentrations were determined and used for reactivity and neutralization screening, if Ig concentration was above 1 μg/ml. For biophysical characterization assays and in vivo experiments, supernatants were purified using Protein G Sepharose beads (GE Healthcare), dialyzed against PBS and sterile-filtered using 0.2 μm filter units (GE Healthcare). For in vivo experiments, mAbs were concentrated using Pierce™ 3K Protein Concentrator PES (Thermo Scientific).

### SARS-CoV-2-S1 ELISA

Screening for SARS-CoV-2-specific mAbs was done by using anti-SARS-CoV-2-S1 IgG ELISAs (EUROIMMUN Medizinische Labordiagnostika AG) according to the manufacturer's protocol. mAb containing cell culture supernatants were pre-diluted 1:5, patient sera 1:100. Optical density (OD) ratios were calculated by dividing the OD at 450 nm by the OD of the calibrator included in the kit. OD ratios of 0.5 were considered reactive.

### RBD ELISA

Binding to the receptor-binding domain (RBD) of S1 was tested in an ELISA. To this end, a fusion protein (RBD-Fc) of the signal peptide of the NMDA receptor subunit GluN1, the RBD-SD1 part of SARS-CoV2-S1 (amino acids 319-591) and the constant region of rabbit IgG1 heavy chain (Fc) was expressed in HEK293T cells and immobilized onto 96-well plates from cell culture supernatant via anti-rabbit IgG (Dianova, 711-005-152) antibodies. Then, human mAbs were applied and detected using horseradish peroxidase (HRP)-conjugated anti-human IgG (Dianova, 709-035-149) and the HRP substrate 1-step Ultra TMB (Thermo Fisher Scientific, Waltham, MA). All S1+ mAbs were screened at a human IgG concentration of 10 ng/mL to detect strong RBD binders and the ones negative at this concentration were re-evaluated for RBD reactivity using a 1:5 dilution of the cell culture supernatants. To test for specificity within the coronavirus family, we expressed and immobilized Fc fusion proteins of the RBD-SD1 regions of SARS-CoV, MERS-CoV and the endemic human coronaviruses HCoV-229E, HCoV-NL63, HCoV-HKU1, and HCoV-OC43 and applied mAbs at 1 μg/ml. The presence of immobilized antigens was confirmed by incubation with HRP-conjugated anti-rabbit IgG (Dianova, 711-036-152). Assays for concentration-dependent RBD binding (FIG. 1E) were developed using 1-step Slow TMB (Thermo Fisher Scientific). EC₅₀ was determined from non-linear regression models using Graph Pad Prism 8.

To evaluate the ability of mAbs to interfere with the binding of ACE2 to SARS-CoV-2 RBD, we expressed ACE2-HA, a fusion protein of the extracellular region of human ACE2 (amino acids 1-615) followed by a His-tag and a hemagglutinin (HA)-tag in HEK293T cells and applied it in a modified RBD-ELISA. Captured RBD-Fc was incubated with mAbs at 0.5 μg/ml for 15 minutes and subsequently with ACE2-HA-containing cell culture supernatant for 1 h. ACE2-HA binding was detected using anti-HA antibody HA.11 (clone 16B12, BioLegend, San Diego, CA, 901515), HRP-conjugated anti-mouse IgG (Dianova, 715-035-150) and 1-step UltraTMB.

For experiments regarding the competition between mAbs for RBD binding, purified monoclonal antibodies were biotinylated using EZ-Link Sulfo-NHS-Biotin (Thermo Fisher) according to the manufacturer's instructions. Briefly, 50-200 μg of purified antibody were incubated with 200-fold molar excess Sulfo-NHS-Biotin for 30 minutes at room temperature. Excess Sulfo-NHS-Biotin was removed by dialysis for 16 hours. Recovery rate of IgG ranged from 60-100%. RBD-Fc captured on ELISA plates was incubated with mAbs at 10 μg/ml for 15 minutes. Then, one volume of biotinylated mcAbs at 100 ng/mL was added and the mixture incubated for additional 15 minutes, followed by detection using HRP-conjugated streptavidin (Roche Diagnostics) and 1-step Ultra TMB. Background by the HRP-conjugated detection antibodies alone was subtracted from all absorbance values.

### Circos Plot of Public Clonotypes

Antibodies which share same V and J gene on both Ig heavy and light chain are considered to be one clonotype. Such clonotypes are considered public if they are identified in different patients. After identification of public clonotypes, they were plotted in a Circos plot using the R package circlize.

### Identification of 18 Strongly Neutralizing Antibodies

To identify the most potent SARS-CoV-2 neutralizing mAb, all 122 S1-reactive mAbs were screened for binding to RBD. 87 were defined as strongly binding to RBD (defined as detectable binding at 10 ng/mL in an RBD ELISA) and then assessed for neutralization of authentic SARS-CoV-2 at 25 and 250 ng/mL using mAb-containing cell culture supernatants. Antibodies were further selected (i) as the strongest neutralizing mAb of the respective donor and/or (ii) with an estimated IC₅₀ of 25 ng/mL or below and/or (iii) with an estimated IC₉₀ of 250 ng/mL or below. These were defined as the 18 most potent antibodies (Top-18) and expressed as purified antibodies for detailed biophysical characterization.

### Surface Plasmon Resonance Measurements

The antigen (SARS-CoV-2 S protein-RBD-mFc, Accrobiosystems) was reversibly immobilized on a C1 sensor chip via anti-mouse IgG. Purified mAbs were injected at different concentrations in a buffer consisting of 10 mM HEPES pH 7.4, 150 mM NaCl, 3 mM EDTA, 0.05% Tween 20. CV-X1-126 and CV38-139 were analyzed in a buffer containing 400 mM NaCl as there was a slight upward drift at the beginning of the dissociation phase due to non-specific binding of to the reference flow. Multi-cycle-kinetics analyses were performed in duplicates except for non-neutralizing CV03-191. Kₐ, K_{d} and K_{D}-values were determined using a monovalent analyte model. Recordings were performed on a Biacore T200 instrument at 25° C.

### Plaque Reduction Neutralization Test

To detect neutralizing activity of SARS-CoV-2-specific mAbs, plaque reduction neutralization tests (PRNT) were done as described before (Wolfel et al., 2020). Briefly, Vero E6 cells (1.6×10⁵ cells/well) were seeded in 24-well plates and incubated overnight. For each dilution step, mAbs were diluted in OptiPro and mixed 1:1 with 200 μl virus (Munich isolate 984) (Wolfel et al., 2020) solution containing 100 plaque forming units. The 400 μl mAb-virus solution was vortexed gently and incubated at 37° C. for 1 hour. Each 24-well was incubated with 200 μl mAb-virus solution. After 1 hour at 37°, the supernatants were discarded and cells were washed once with PBS and supplemented with 1.2% Avicel solution in DMEM. After 3 days at 37° C., the supernatants were removed and the 24-well plates were fixed and inactivated using a 6% formaldehyde/PBS solution and stained with crystal violet. All dilutions were tested in duplicates. For PRNT-screening mAb dilutions of 25 and 250 ng of IgG/ml were assessed. IC₅₀ was determined from non-linear regression models using Graph Pad Prism 8.

### Immunocytochemistry

Recombinant spike protein-based immunofluorescence assays were done as previously described (Buchholz et al., 2013; Corman et al., 2020; Wolfel et al., 2020). Briefly, VeroB4 cells were transfected with previously described pCG1 plasmids encoding SARS-CoV-2, MERS-CoV, HCoV-NL63, -229E, -OC43, and -HKU1 spike proteins (Buchholz et al., 2013; Hoffmann et al., 2020). For transfection, Fugene HD (Roche) was used in a Fugene to DNA ratio of 3:1. After 24 hours, transfected as well as untransfected VeroB4 cells were harvested and resuspended in DMEM/10% FCS to achieve a cell density of 2.5×10⁵ cells/ml each. Transfected and untransfected VeroB4 cells were mixed 1:1 and 50 μl of the cell suspension was applied to each incubation field of a multitest cover slide (Dunn Labortechnik). The multitest cover slides were incubated for 6 hours before they were washed with PBS and fixed with ice-cold acetone/methanol (ratio 1:1) for 10 minutes. For the immunofluorescence test, the incubation fields were blocked with 5% non-fat dry milk in PBS/0.2% Tween for 60 minutes. Purified mAbs were diluted in EUROIMMUN sample buffer to a concentration of 5 μg/ml and 30 μl of the dilution was applied per incubation field. After 1 hour at room temperature, cover slides were washed 3 times for 5 minutes with PBS/0.2% Tween. Secondary detection was done using a 1:200 dilution of a goat-anti human IgG-Alexa488 (Dianova). After 30 minutes at room temperature, slides were washed 3 times for 5 minutes and rinsed with water. Slides were mounted using DAPI prolonged mounting medium (FisherScientific).

### Murine Tissue Reactivity Screening

Preparations of brain, lung, heart, liver, kidney and gut from 8-12 weeks old C57BL/6J mice were frozen in −50° C. 2-methylbutane, cut on a cryostat in 20 μm sections and mounted on glass slides. For tissue reactivity screening according to established protocols (Kreye et al., 2016), thawed unfixed tissue slices were rinsed with PBS then blocked with blocking solution (PBS supplemented with 2% Bovine Serum Albumin (Roth) and 5% Normal Goat Serum (Abcam)) for 1 hour at room temperature before incubation of mAbs at 5 μg/ml overnight at 4° C. After three PBS washing steps, goat anti-human IgG-Alexa Fluor 488 (Dianova, 109-545-003) diluted in blocking solution was applied for 2 hours at room temperature before additional three washes and mounting using DAPI-containing Fluoroshield. Staining was examined under an inverted fluorescence microscope (Olympus CKX41, Leica DMI6000) or confocal device (Leica TCS SL). For co-staining, tissue was processed as above, but sections were fixed with 4% PFA in PBS for 10 minutes at room temperature before blocking. For co-staining, the following antibodies were used: mouse Smooth Muscle Actin (clone 1A4, Agilent, 172 003), goat anti-mouse IgG-Alexa Fluor 594 (Dianova, 115-585-003). For nuclei staining DRAQ5™ (abcam, ab108410) was used.

### HEp2 Cell Assay

HEp-2 cell reactivity was investigated using the NOVA Lite HEp-2 ANA Kit (Inova Diagnostics) according to the manufacturer's instructions using mAb containing culture supernatant (screening of all S1+ mAbs) or purified mAbs at 50 μg/ml (polyreactivity testing of CV07-200, CV07-209, CV07-222, CV07-255, CV07-270 and CV38-148) and examined under an inverted fluorescence microscope.

### Polyreactivity Screening ELISA

Purified mAbs were screened for reactivity against cardiolipin and beta-2 microglobulin at 50 μg/ml using routine laboratory ELISAs.

### Hamster Model of SARS-CoV-2 Infection

Virus stocks for animal experiments were prepared from the previously published SARS-CoV-2 München isolate (Wolfel et al., 2020). Viruses were propagated on Vero E6 cells (ATCC CRL-1586) in minimal essential medium (MEM; PAN Biotech) supplemented with 10% fetal bovine serum (PAN Biotech) 100 IU/ml Penicillin G and 100 μg/ml Streptomycin (Carl Roth). Stocks were stored at −80° C. prior to experimental infections.

For the SARS-CoV-2 challenge experiments, hamsters were randomly distributed into three groups: In the first group (prophylaxis group), animals received an intraperitoneal (i.p.) injection of 18 mg per kg bodyweight of SARS-CoV-2 neutralizing mAb CV07-209 24 hours prior to infection. In the second and third group (treatment and control group, respectively), animals were given the identical mAb amount two hours after infection, either with 18 mg/kg of CV07-209 (treatment group) or with 20 mg/kg of non-reactive isotype-matched mGO53 (control group). Hamsters were infected intranasally with 1×10⁵ PFU SARS-CoV-2 diluted in minimal essential medium (MEM; PAN Biotech) to a final volume of 60 μl as previously described (Osterrieder et al., 2020).

On days 2, 5 and 13 post infection, three hamsters of each group were euthanized by exsanguination under general anesthesia employing a protocol developed specifically for hamsters and consisting of 0.15 mg/kg medetomidine, 2 mg/kg midazolam and 2.5 mg/kg butorphanol applied as a single intramuscular injection of 200 μl (Nakamura et al., 2017). Nasal washes, tracheal swabs, and lungs (left and right) were collected for histopathological examinations and/or virus titrations and RT-qPCR. Body weights were recorded daily and clinical signs of all animals were monitored twice daily throughout the experiment.

### Histopathology and In Situ Hybridization

For histopathological examination and in situ hybridization (ISH) of lung tissues, the left lung lobe was carefully removed and immersed in fixative solution (4% formalin, pH 7.0) for 48 hours. Lungs were then embedded in paraffin and cut in 2 μm sections. For histopathology, slides were stained with hematoxylin and eosin (HE) after dewaxing in xylene and rehydration in decreasing ethanol concentrations. Lung sections were microscopically evaluated in a blinded fashion by board-certified veterinary pathologists to assess the character and severity of pathologic lesions using lung-specific inflammation scoring parameters as previously described for SARS-Cov2 infection in hamsters (Osterrieder et al., 2020). These parameters included severity of interstitial pneumonia, immune cell infiltration by neutrophils, macrophages, and lymphocytes, bronchitis, epithelial necrosis of bronchi and alveoli, hyperplasia of bronchial epithelial cells and type II-alveolar epithelial cells, endothelialitis, perivascular lymphocytic cuffing, as well as alveolar edema and perivascular edema. The following parameters were evaluated to assess three different scores: (1) the bronchitis score that includes severity of bronchial inflammation and epithelial cell necrosis of bronchi, (2) the regeneration score including hyperplasia of bronchial epithelial cells and type-II-alveolar epithelial cells, and (3) the edema score including alveolar and perivascular edema.

ISH was performed as reported previously (Osterrieder et al., 2020) using the ViewRNA™ ISH Tissue Assay Kit (Invitrogen by Thermo Fisher Scientific) following the manufacturer's instructions with the minor following adjustments. Probes for the detection of the Nucleoprotein (N) gene RNA of SARS-CoV-2 (NCBI database NC_045512.2, nucleotides 28,274 to 9,533, assay ID: VPNKRHM), and the murine housekeeping gene eukaryotic translation elongation factor-1α (EF1a; assay ID: VB1-14428-VT, Affymetrix, Inc.), that shares 95% sequence identity with the Syrian hamster, were designed. Lung sections (2 μm thickness) on adhesive glass slides were dewaxed in xylol and dehydrated in ethanol. Tissues were incubated at 95° C. for 10 minutes with subsequent protease digestion for 20 minutes. Sections were fixed with 4% paraformaldehyde in PBS (Alfa Aesar, Thermo Fisher) and hybridized with the probes. Amplifier and label probe hybridizations were performed according to the manufacturer's instructions using fast red as the chromogen, followed by counterstaining with hematoxylin for 45 s, washing in tap water for 5 minutes, and mounting with Roti@-Mount Fluor-Care DAPI (4, 6-diaminidino-2-phenylindole; Carl Roth). An irrelevant probe for the detection of pneumolysin was used as a control for sequence-specific binding. HE-stained and ISH slides were analyzed and images taken using a BX41 microscope (Olympus) with a DP80 Microscope Digital Camera and the cellSens™ Imaging Software, Version 1.18 (Olympus).

### Virus Titrations, RNA Extractions and RT-qPCR

To determine virus titers from 25 mg lung tissue, tissue homogenates were serially diluted and titrated on Vero E6 cells in 12-well-plates. Three days later, cells were formalin-fixed, stained with crystal violet and plaques were counted. RNA was extracted from homogenized lungs, nasal washes and tracheal swabs using the innuPrep Virus DNA/RNA Kit (Analytik Jena) according to the manufacturer's instructions. We quantified RNA using a one-step RT qPCR reaction with the NEB Luna Universal Probe One-Step RT-qPCR kit (New England Biolabs) by following the manufacturer's instructions and by using previously published TaqMan primers and probes (SARS-CoV-2 E_Sarbeco and hamster RPL18) (Corman et al., 2020; Zivcec et al., 2011) on a StepOnePlus RealTime PCR System (Thermo Fisher Scientific).

Detection of subgenomic RNA (sgRNA) was done using by oligonucleotides targeting the leader transcriptional regulatory sequence and by oligonucleotides targeting regions within the E gene, as described previously. The sgRNA RT-PCR assay used the Platinum™ SuperScript™ III RT-PCR-System with Platinum Taq DNA Polymerase (Thermo Fisher Scientific). A 25 μL reaction contained 5 μL of RNA, 12.5 μL of 2× reaction buffer provided with the kit (containing 0.4 mM of each deoxyribont triphosphates (dNTP) and 3.2 mM magnesium sulphate), 1 μL of reverse transcriptase/Taq mixture from the kit, 1 μg of nonacetylated bovine serum albumin (Roche), and 0.4 μL of a 50 mM magnesium sulphate solution (Thermo Fisher Scientific).

### Quantification and Statistical Analysis

All statistical tests were performed using GraphPad Prism, version 8.4. For comparison of SHM number, a D'Agostino-Pearson normality test showed that the number of SHM was not normally distributed, therefore a Kruskal-Wallis test was used with posthoc Dunn's multiple comparisons tests. For bodyweight changes from hamster experiments a D'Agostino-Pearson normality test revealed normal distribution, Thus, statistical significance of bodyweight changes from hamster experiments was tested using a mixed-effects model (two-way ANOVA) with posthoc Dunnett's multiple comparisons test in comparison to control group. Statistical details can be found in the figure legends. For Top-18 mAbs, EC₅₀ and IC₅₀ values were determined from non-linear regression models using Graph Pad Prism 8.4. Binding kinetics of mAbs to RBD were modeled from multi-cycle surface plasmon resonance measurements using the Biacore T200 software, version 3.2.

### Measurement of Binding Affinities and Competition Using Biolayer Interferometry

Binding assays were performed by biolayer interferometry (BLI) using an Octet Red instrument (FortéBio). To determine the binding affinity of CV38-142 Fab with SARS-CoV-2 and SARS-CoV RBDs, 20 μg/mL of His-tagged SARS-CoV or SARS-CoV-2 RBD protein purified from Hi5 cell expression was diluted in kinetics buffer (1× PBS, pH 7.4, 0.002% Tween-20, 0.01% BSA) and loaded on Ni-NTA biosensors (ForteBio) for 300 s. After equilibration in kinetics buffer for 60 s, the biosensors were transferred to wells containing serially diluted Fab samples in running buffer to record the real time association response signal. After a 120 s association step, the biosensors were transferred to wells containing blank running buffer to record the real time disassociation response signal. All steps were performed at 1000 r.p.m. shaking speed. K_{D}s were determined using ForteBio Octet CFR software. To determine the binding affinity of CV38-142 Fab or S309 IgG with SARS-CoV-2 RBD pretreated with or without PNGase F, Fab or IgG was loaded on Fab2G or AHC biosensors (ForteBio) for 300 s followed by similar steps to test binding to RBD that was expressed in Expi293F cells. For the sandwich binning assay, CV38-142 IgG was loaded onto AHC biosensors (ForteBio) followed by equilibration in kinetics buffer. The biosensors were transferred to wells containing either SARS-CoV-2 RBD or S-HexaPro proteins in kinetics buffer to allow for antigen association for 200 s followed by testing association of a second antibody Fab or ACE2 for 120 s.

### Measurement of Competition Using Surface Plasma Resonance

To test whether binding of CV38-142 to SARS-CoV-2 RBD has an impact on the binding of ACE2, a surface plasma resonance (SPR) competition assay was performed on a Biacore T200 instrument (Cytiva) at 25° C. Biotinylated human ACE2 (residue 18-740, ACROBiosystems) was reversibly immobilized on a CAP sensor chip (Cytiva) using Biotin CAPture Kit (Cytiva). CV38-142 IgG used in the SPR assay was produced in CHO cells and was kindly provided by Miltenyi Biotec, Bergisch Gladbach, Germany. The SPR system was primed and equilibrated with running buffer (10 mM HEPES pH 7.4, 150 mM NaCl, 3 mM EDTA, 0.05% Tween 20) before measurement. 10 nM of SARS-CoV-2 RBD (ACROBiosystems) together with different concentrations of CV38-142 IgG dissolved in the running buffer were injected into the system within 90 s in a flow rate of 30 μl/min followed by a regeneration step between each concentration. The binding response signals were recorded in real time by subtracting from reference cell. And the experiment was repeated once.

### Enzyme-Linked Immunosorbent Assay (ELISA) Measuring Antibody Binding to RBD

Rabbit IgG1 Fc-tagged RBD-SD1 regions of MERS-CoV, SARS-CoV and SARS-CoV-2 as well as point mutants thereof (SARS-CoV: N330Q and T332A, SARS-CoV-2: N343Q and T345A) were expressed in HEK293T cells and immobilized onto 96-well plates as previously described. Mutations were introduced by overlap extension PCR and confirmed by Sanger sequencing (LGC Genomics). Human anti-spike RBD monoclonal antibodies were applied at 1 μg/ml and detected using horseradish peroxidase (HRP)-conjugated anti-human IgG (Dianova) and the HRP substrate 1-step Ultra TMB (Thermo Fisher Scientific). HRP-conjugated F (ab')2 anti-rabbit IgG (Dianova) was used to confirm the presence of immobilized antigens.

### Pseudovirus Neutralization Assay and Synergistic Study

Pseudovirus preparation and assay were performed as previously described with minor modifications. Pseudovirions were generated by co-transfection of HEK293T cells with plasmids encoding MLV-gag/pol, MLV-CMV-Luciferase, and SARS-CoV-2418 spike (GenBank: MN908947) or SARS-CoV spike (GenBank: AFR58672.1). The cell culture supernatant containing SARS-CoV-2 and SARS-CoV S-pseudotyped MLV virions was collected at 48 hours post transfection and stored at −80° C. until use. Lentivirus transduced Hela cells expressing hACE2 (GenBank: BAB40370.1) were enriched by fluorescence-activated cell sorting (FACS) using biotinylated SARS-CoV-2 RBD conjugated with streptavidin-Alexa Fluor 647 (Thermo, S32357). Monoclonal antibodies IgG or Fab were serially diluted with DMEM medium supplemented with 10% heat-inactivated FBS, 1% Q-max, and 1% P/S. The serial dilutions were incubated with the pseudotyped viruses at 37° C. for 1 hour in 96-well half-well plate (Corning, 3688). After the incubation, 10,000 Hela-hACE2 cells were added to the mixture and supplemented 20 μg/ml Dextran (Sigma, 93556-1G) for enhanced infectivity. The supernatant was removed 48 hours post incubation, and the cells were washed and lysed in luciferase lysis buffer (25 mM Gly-Gly PH 7.8, 15 mM MgSO₄, 4 mM EGTA, 1% V/V Triton X-100). After addition of Bright-Glo (Promega, PR-E2620) according to the manufacturer's instruction, luminescence signal was measured in duplicate. At least two biological replicates were performed for each assay. The IgG half-maximal inhibitory concentration (IC₅₀) values were calculated using “One Site-Fit LogIC50” regression in GraphPad Prism 9. For synergy assessment of two monoclonal antibodies, an antibody cocktail matrix was prepared by a combination of mixing a fixed concentration of CV38-142 and increasing the concentration of COVA1-16 or increasing the concentration of CV38-142 and fixing the concentration of COVA1-16. Neutralization percentages for each combination were measured and calculated the same way as the pseudovirus neutralization assay. The neutralization data were converted to the input format for the synergy program. Synergy scores were calculated by fitting the multidimensional synergy of combinations (MuSyC) model, which is a synergy model based on a multidimensional extension of the Hill equation that allows non-linear dose-response surface contour. MuSyC model quantifies synergy in bidirectional way and distinguishes synergies between potency and efficacy. The synergy parameter α₁₂, namely synergistic potency quantifies how the second antibody changes the first's potency and α₂₁ quantifies how the first changes the second's potency. The MuSyC model fitting with the synergy program also gives two other parameters, namely synergistic efficacy (β) and synergistic cooperativity (γ) score. The β score denotes synergistic efficacy, which quantifies the percent change on the maximal efficacy of the antibody combination compared to the most efficacious single agent. The γ₁₂ score denotes how the first antibody changes the second's Hill slop while γ₂₁ denotes how the second changes the first's Hill slop.

### REFERENCES

Kreye, J., et al Human cerebrospinal fluid monoclonal N-methyl-D-aspartate receptor autoantibodies are sufficient for encephalitis pathogenesis. Brain 139, 2641-2652 (2016).
Kornau, H. C., et al Human Cerebrospinal Fluid Monoclonal LGI1 Autoantibodies Increase Neuronal Excitability. Ann Neurol 87, 405-418 (2020).
Ju, B. et al. Potent human neutralizing antibodies elicited by SARS-CoV-2 infection. bioRxiv 2020.03.21.990770; doi: https://doi.org/10.1101/2020.03.21.990770 [Pre-print—not peer reviewed]
Reincke S. M., et al. Brain Antibody Sequence Evaluation (BASE): an easy-to-use software for complete data analysis in single cell immunoglobulin cloning. bioRxiv 2019.11.09; doi: https://doi.org/10.1101/836999
Hoffmannm M., et al. SARS-CoV-2 Cell Entry Depends on ACE2 and TMPRSS2 and Is Blocked by a Clinically Proven Protease Inhibitor Cell April 16; 181(2):271-280.e8. (2020).
Jiang S., et al. Neutralizing Antibodies against SARS-CoV-2 and Other Human Coronaviruses, Trends in Immunology, May 2020, Vol. 41, No. 5, p. 355
Ou, X., et al. Characterization of spike glycoprotein of SARS-CoV-2 on virus entry and its immune cross-reactivity with SARS-CoV. Nat Commun 11, 1620 (2020)
Jin Y., et al. Virology, Epidemiology, Pathogenesis, and Control of COVID-19, Viruses 2020, 12, 372
Wang and Weiner, Complement and Cellular Cytotoxicity in Antibody Therapy of Cancer, Expert Opin Biol Ther 8:759-768 (2008)
Kabat et al. Sequences of Proteins of Immunological Interest, (5th ed., 1991, National Institutes of Health, Bethesda Md.))
Al-Iazikani et al. (1997) Standard Conformations for the Canonical Structures of Immunoglobulins. J. Molec. Biol. 273:927-948
Nimmerjahn and Ravetch (2008) Fcgamma Receptors as Regulators of Immune Responses, Nat Rev Immunol 8:34-47;
Chavez-Galan et al. (2009) Cell Death Mechanisms Induced by Cytotoxic Lymphocytes, Cell Mol Immunol 6:15-25).
Robbiani, et al, Convergent Antibody Responses to SARS-CoV-2 Infection in Convalescent Individuals, doi: https://doi.org/10.1101/2020.05.13.092619 [Pre-print-not peer reviewed]
Cao et al, Potent neutralizing antibodies against SARS-CoV-2 identified by high-throughput single-cell sequencing of convalescent patients' B cells, Cell. 2020 May 18; doi: 10.1016/j.cell.2020.05.025 [Epub ahead of print]
Chunyan Wang et al, “A human monoclonal antibody blocking SARS-CoV-2 infection”, bioRxiv, 12 Mar. 2020.
Wu Yanling et al, “Identification of Human Single-Domain Antibodies against SARS-CoV-2”, Cell Host & Microbe, vol. 27, no. 6, 14 May 2020.
Chen Xiangyu et al, “Human monoclonal antibodies block the binding of SARS-CoV-2 spike protein to angiotensin converting enzyme 2 receptor”, Cellular & Molecular Immunology, vol. 17, no. 6, 20 Apr. 2020.
Shi Rui et al, “A human neutralizing antibody targets the receptor-binding site of SARS-CoV-2”, Nature, vol. 584, no. 7819, 26 May 2020.
Seth J. Zost et al, “Potently neutralizing human antibodies that block SARS-CoV-2 receptor binding and protect animals”, bioRxiv, 22 May 2020.
Osterrieder, N., Bertzbach, L. D., Dietert, K., Abdelgawad, A., Vladimirova, D., Kunec, D., Hoffmann, D., Beer, M., Gruber, A. D., and Trimpert, J. (2020). Age-Dependent Progression of SARS-CoV-2 Infection in Syrian Hamsters. Viruses 12.

1. A human recombinant monoclonal antibody or antibody fragment that binds the receptor binding domain (RBD) of the S1 subunit (S1) of a recombinant Spike glycoprotein of SARS-CoV-2 expressed from human embryonic kidney (HEK293) cells (HEK-SARS-CoV-2-Spike-S1-RBD glycoprotein), wherein the heavy and light chain variable amino acid sequences of the antibody (VH and VL) were isolated from convalescent COVID-19 patients.

2. The antibody or antibody fragment according to claim 1, wherein the heavy and light chain variable amino acid sequences of the antibody (VH and VL) were isolated from CD19+CD27+CD38+ antibody-secreting cells (ASCs) and/or CD19+CD27+ memory B cells (MBCs) of convalescent COVID-19 patients that bind the HEK-SARS-CoV-2-Spike-S1-RBD glycoprotein.

3. The antibody or antibody fragment according to claim 1, wherein the HEK-SARS-CoV-2-Spike-S1-RBD glycoprotein comprises or consists of an amino acid sequence according to SEQ ID NO 3.

4. The antibody or antibody fragment according to claim 1, wherein the antibody or antibody fragment inhibits the interaction between angiotensin-converting enzyme 2 (ACE2) or fragment thereof according to SEQ ID NO 7 with the SARS-CoV-2-Spike-S1-RBD.

5. The antibody or antibody fragment according to claim 4, wherein the inhibition of interaction between a soluble fragment of ACE2 according to SEQ ID NO 7 and SARS-CoV-2-Spike-S1-RBD according to SEQ ID NO 3 obtained by the antibody or antibody fragment is at least 20 in an in vitro competition assay in which HEK-SARS-CoV-2-Spike-S1-RBD glycoprotein is immobilized on a solid phase and incubated with the antibody or antibody fragment and subsequently with ACE2.

6. The antibody or antibody fragment according to claim 1, wherein the antibody or antibody fragment inhibits the infection of epithelial cells with SARS-CoV-2.

7. The antibody or antibody fragment according to claim 6, wherein the antibody or antibody fragment has an IC50 of <2000 ng/mL, in a plaque reduction neutralization test (PRNT).

8. The antibody or antibody fragment according to claim 1, wherein the antibody or antibody fragment does not bind, or binds at negligible levels, unfixed mammalian tissue sections in vitro, said sections selected from one or more of brain, heart, kidney and/or lung tissue sections.

9. The antibody or antibody fragment according to claim 1, comprising:
a heavy chain (variable VH) domain, said VH domain comprising complementary determining region (CDR) sequences of:
a. H-CDR1 selected from SEQ ID NO 10, 18, 26, 34, 42, 50, 58, 66, 74, 82, 90, 98, 106, 114, 122, 130, 138, 146 or 154,
b. H-CDR2 selected from SEQ ID NO 11, 19, 27, 35, 43, 51, 59, 67, 75, 83, 91, 99, 107, 115, 123, 131, 139, 147 or 155,
c. H-CDR3 selected from SEQ ID NO 12, 20, 28, 36, 44, 52, 60, 68, 76, 84, 92, 100, 108, 116, 124, 132, 140, 148 or 156,
and a light chain variable (VL) domain, said VL domain comprising complementary determining region (CDR) sequences of:
a. L-CDR1 selected from SEQ ID NO 13, 21, 29, 37, 45, 53, 61, 69, 77, 85, 93, 101, 109, 117, 125, 133, 141, 149 or 157,
b. L-CDR2 selected from SEQ ID NO 14, 22, 30, 38, 46, 54, 62, 70, 78, 86, 94, 102, 110, 118, 126, 134, 142, 150 or 158,
c. L-CDR3 selected from SEQ ID NO 15, 23, 31, 39, 47, 55, 63, 71, 79, 87, 95, 103, 111, 119, 127, 135, 143, 151 or 159.

10. The antibody or antibody fragment according to claim 9, comprising a heavy chain variable (VH) domain, said VH domain comprising a sequence of at least 70% identity to one of SEQ ID NO 16, 24, 32, 40, 48, 56, 64, 72, 80, 88, 96, 104, 112, 120, 128, 136, 144, 152 or 160, and a light chain variable (VL) domain, said VL domain comprising a sequence of at least 70% identity to one of SEQ ID NO 17, 25, 33, 41, 49, 57, 65, 73, 81, 89, 97, 105, 113, 121, 129, 137, 145, 153 or 161.

11. The antibody or antibody fragment according to claim 9, comprising VH and VL domains that comprise the sequences according to SEQ ID NO 16 and 17, 24 and 25, 32 and 33, 40 and 41, 48 and 49, 56 and 57, 64 and 65, 72 and 73, 80 and 81, 88 and 89, 96and 97, 104 and 105, 112 and 113, 120 and 121, 128 and 129, 136 and 137, 144 and 145, 152 and 153, and 160 and 161, respectively.

12. A nucleic acid molecule comprising a nucleotide sequence that encodes an antibody or antibody fragment according to claim 1.

13. A host cell, that produces an antibody or antibody fragment according to claim 1.

14. A pharmaceutical composition comprising the isolated antibody or antibody fragment according to claim 1, with a pharmaceutically acceptable carrier.

15. A method of treating a medical condition associated with a SARS Coronavirus, comprising administering an antibody or antibody fragment according to claim 1 to a patient in need thereof.

16. An in vitro method for determining the presence or absence of SARS-CoV-2 viral protein in a sample, comprising contacting an antibody or antibody fragment according to claim 1 with the sample to enable formation of an antibody-SARS-CoV-2 complex and subsequent detecting of said antibody-SARS-CoV-2 complex if present in said sample.

17. The method according to claim 15, wherein the medical condition associated with a SARS Coronavirus is COVID-19 or a SARS Coronavirus-associated respiratory disease.

18. The antibody or antibody fragment according to claim 9, wherein the antibody or antibody fragment comprises 6 CDRs according to:
SEQ ID NO 10-15, 18-23, 26-31, 34-39, 42-47, 50-55, 58-63, 66-71, 74-79, 82-87, 90-95, 98-103, 106-111, 114-119, 122-127, 130-135, 138-143, 146-151 or 154-159.

19. The antibody or antibody fragment according to claim 9, wherein the antibody or antibody fragment comprises:
a VH sequence with at least 80% identity to SEQ ID NO 16 and a VL sequence with at least 80% identity to SEQ ID NO 17, and at least 6 CDRs according to SEQ ID NO 10-15,
a VH sequence with at least 80% identity to SEQ ID NO 24 and a VL sequence with at least 80% identity to SEQ ID NO 25, and at least 6 CDRs according to SEQ ID NO 18-23,
a VH sequence with at least 80% identity to SEQ ID NO 32 and a VL sequence with at least 80% identity to SEQ ID NO 33, and at least 6 CDRs according to SEQ ID NO 26-31,
a VH sequence with at least 80% identity to SEQ ID NO 40 and a VL sequence with at least 80% identity to SEQ ID NO 41, and at least 6 CDRs according to SEQ ID NO 34-39,
a VH sequence with at least 80% identity to SEQ ID NO 48 and a VL sequence with at least 80% identity to SEQ ID NO 49, and at least 6 CDRs according to SEQ ID NO 42-47,
a VH sequence with at least 80% identity to SEQ ID NO 56 and a VL sequence with at least 80% identity to SEQ ID NO 57, and at least 6 CDRs according to SEQ ID NO 50-55,
a VH sequence with at least 80% identity to SEQ ID NO 64 and a VL sequence with at least 80% identity to SEQ ID NO 65, and at least 6 CDRs according to SEQ ID NO 58-63,
a VH sequence with at least 80% identity to SEQ ID NO 72 and a VL sequence with at least 80% identity to SEQ ID NO 73, and at least 6 CDRs according to SEQ ID NO 66-71,
a VH sequence with at least 80% identity to SEQ ID NO 80 and a VL sequence with at least 80% identity to SEQ ID NO 81, and at least 6 CDRs according to SEQ ID NO 74-79,
a VH sequence with at least 80% identity to SEQ ID NO 88 and a VL sequence with at least 80% identity to SEQ ID NO 89, and at least 6 CDRs according to SEQ ID NO 82-87,
a VH sequence with at least 80% identity to SEQ ID NO 96 and a VL sequence with at least 80% identity to SEQ ID NO 97, and at least 6 CDRs according to SEQ ID NO 90-95,
a VH sequence with at least 80% identity to SEQ ID NO 104 and a VL sequence with at least 80% identity to SEQ ID NO 105, and at least 6 CDRs according to SEQ ID NO 98-103,
a VH sequence with at least 80% identity to SEQ ID NO 112 and a VL sequence with at least 80% identity to SEQ ID NO 113, and at least 6 CDRs according to SEQ ID NO 106-111,
a VH sequence with at least 80% identity to SEQ ID NO 120 and a VL sequence with at least 80% identity to SEQ ID NO 121, and at least 6 CDRs according to SEQ ID NO 114-119,
a VH sequence with at least 80% identity to SEQ ID NO 128 and a VL sequence with at least 80% identity to SEQ ID NO 129, and at least 6 CDRs according to SEQ ID NO 122-127,
a VH sequence with at least 80% identity to SEQ ID NO 136 and a VL sequence with at least 80% identity to SEQ ID NO 137, and at least 6 CDRs according to SEQ ID NO 130-135,
a VH sequence with at least 80% identity to SEQ ID NO 144 and a VL sequence with at least 80% identity to SEQ ID NO 145, and at least 6 CDRs according to SEQ ID NO 138-143,
a VH sequence with at least 80% identity to SEQ ID NO 152 and a VL sequence with at least 80% identity to SEQ ID NO 153, and at least 6 CDRs according to SEQ ID NO 146-151,
or
a VH sequence with at least 80% identity to SEQ ID NO 160 and a VL sequence with at least 80% identity to SEQ ID NO 161, and at least 6 CDRs according to SEQ ID NO 154-159.
